(19) United States
(12) Patent Application Publication
(10) Pub. No.: US 2026/0176307 A1
(43) Pub. Date: Jun. 25, 2026

(54) ANTI-TDP-43 ANTIBODIES AND USES THEREOF

(71) Applicant: VectorY Therapeutics B.V., Amsterdam (NL)

(72) Inventors: Pavlina KONSTANTINOVA, Amsterdam (NL); Sander VAN DEVENTER, Amsterdam (NL); Wouter POS, Amsterdam (NL); Svitlana PASTEUNING-VUHMAN, Amsterdam (NL)

(21) Appl. No.: 19/555,333

(22) Filed: Mar. 3, 2026

Related U.S. Application Data
(63) Continuation of application No. PCT/IB2024/000774, filed on Dec. 19, 2024.
(60) Provisional application No. 63/611,860, filed on Dec. 19, 2023.

(51) Int. Cl. C07K 14/005; C12N 15/86

(52) U.S. Cl. CPC C07K 14/005; C12N 15/86; C07K 2317/24

(57) ABSTRACT

Provided herein are antibodies and polypeptides that specifically bind to TDP-43. Also provided are pharmaceutical compositions comprising these antibodies, polynucleotides encoding these antibodies, expression vectors and host cells for making these antibodies, and methods of treating a subject using these antibodies.

### RELATED APPLICATIONS

This application is a continuation of PCT Application No. PCT/IB2024/000774, filed Dec. 19, 2024, which claims benefit of and priority to U.S. Provisional Application No. 63/611,860 filed on Dec. 19, 2023, the contents of which are incorporated by reference herein in its entirety.

### SEQUENCE LISTING

The content of the electronically submitted Sequence Listing XML (Name: VEBV_007_01US_SeqList_ST26.xml; Size: 319,408 bytes; and Created on Mar. 2, 2026) is incorporated by reference herein in its entirety.

### BACKGROUND

Transactive response DNA binding protein of 43 kDa (TDP-43), a 414-amino acid protein encoded by the TARDBP gene, is a widely expressed member of the heterogeneous nuclear ribonucleoprotein family. In normal cells, its functional RNA Recognition Motif (RRM) domains enable RNA processing, transcriptional regulation, and miRNA synthesis. TDP-43 contains a nuclear localization sequence (NLS) and a nuclear export signal (NES) allowing it to shuttle between the nucleus and cytoplasm. Mutations in the TARDBP gene are associated with neurodegenerative conditions such as familial amyotrophic lateral sclerosis (ALS), and TDP-43 pathology has also been associated with sporadic ALS, frontotemporal dementia (FTD), Alzheimer's disease, Parkinson's disease (PD), Inclusion body myositis (IBM), Oculopharyngeal muscular dystrophy (OPMD), Chronic Traumatic Encephalopathy (CTE), and limbic predominant age-related TDP-43 encephalopathy (LATE). This pathology involves abnormal modifications, including hyperphosphorylation, ubiquitination, and TDP-43 protein aggregates. In ALS, TDP-43 accumulates in motor neurons and glia, leading to the loss of motor functions, respiratory failure and, ultimately, death. The spread of TDP-43 across neurons has been shown to occur through a prion-like mechanism, involving cell-to-cell propagation of pathological protein aggregates.

Accordingly, there is a need for therapies targeting TDP-43.

### SUMMARY

The present disclosure provides antibodies and polypeptides that specifically bind to TDP-43 (e.g., human TDP-43). Also provided are pharmaceutical compositions comprising these antibodies, polynucleotides encoding these antibodies, expression vectors and host cells for making these antibodies, and methods of treating a subject using these antibodies. The antibodies disclosed herein are particularly advantageous in that they are able to prevent and/or reduce TDP-43 aggregation, mislocation of TDP-43 from the cell nucleus, and stress-induced neuronal dysfunction.

Accordingly, in one aspect, provided herein is an antibody that specifically binds TDP-43, the antibody comprising: a VH comprising the CDRH1, CDRH2, and CDRH3 amino acid sequences of any one of the VH amino acid sequences set forth in SEQ ID NOs: 66-81; and a VL comprising the CDRL1, CDRL2, and CDRL3 amino acid sequences of any one of the VL amino acid sequences set forth in SEQ ID NOs: 82-105.

In certain embodiments, the antibody comprises the CDRH1, CDRH2, CDRH3, CDRL1, CDRL2, and CDRL3 amino acid sequences of the VH and VL amino acid sequences, respectively, set forth in SEQ ID NOs: 66 and 82; 67 and 83; 68 and 84; 69 and 85; 68 and 86; 68 and 87; 70 and 88; 71 and 89; 72 and 90; 73 and 91; 74 and 92; 75 and 93; 76 and 94; 76 and 95; 77 and 96; 76 and 97; 78 and 98; 76 and 99; 79 and 100; 76 and 101; 76 and 102; 76 and 103; 80 and 104; or 81 and 105.

In certain embodiments, the antibody comprises the CDRH1, CDRH2, CDRH3, CDRL1, CDRL2, and CDRL3 amino acid sequences, respectively, set forth in SEQ ID NOs: 6, 2, ES, 7, 8 and 9; 10, 23, ES, 7, 11 and 16; 35, 48, 41, 45, 28 and 49; 1, 2, KS, 3, 4 and 5; 10, 2, ES, 7, 11 and 9; 10, 12, ES, 7, 13 and 14; 10, 2, ES, 7, 15 and 16; 10, 2, ES, 17, 18 and 19; 10, 2, ES, 7, 20 and 16; 10, 12, ES, 21, 22 and 16; 24, 25, 26, 27, 28 and 29; 30, 31, 32, 3, 33 and 34; 35, 36, 37, 38, 39 and 40; 35, 31, 41, 42, 43 and 44; 35, 31, 41, 45, 46 and 47; 35, 31, 41, 45, 50 and 47; 35, 51, 41, 52, 53 and 49; 35, 31, 41, 45, 28 and 47; 35, 54, 41, 45, 55 and 56; 35, 31, 41, 45, 57 and 47; 35, 31, 41, 58, 50 and 49; 35, 31, 41, 45, 59 and 49; 35, 60, 61, 3, 62 and 63; or 64, 65, 41, 45, 50 and 49. In certain embodiments, the antibody comprises the CDRH1, CDRH2, CDRH3, CDRL1, CDRL2, and CDRL3 amino acid sequences, respectively, set forth in SEQ ID NOs: 6, 2, ES, 7, 8, and 9. In certain embodiments, the antibody comprises the CDRH1, CDRH2, CDRH3, CDRL1, CDRL2, and CDRL3 amino acid sequences, respectively, set forth in SEQ ID NOs: 10, 23, ES, 7, 11 and 16. In certain embodiments, the antibody comprises the CDRH1, CDRH2, CDRH3, CDRL1, CDRL2, and CDRL3 amino acid sequences, respectively, set forth in SEQ ID NOs: 35, 48, 41, 45, 28 and 49.

In certain embodiments, the VH and VL comprise human or human-derived framework regions. In certain embodiments, the VH comprises IGHV1-46 framework sequences. In certain embodiments, the VL comprises IGKV2-28, IGKV2D-28, IGKV2-29, IGKV2D-29, IGKV2-40, or IGKV2D-40 framework sequences.

In certain embodiments, the VH comprises the amino acid sequence of any one of SEQ ID NOs: 66-81.

In certain embodiments, the VL comprises the amino acid sequence of any one of SEQ ID NOs: 82-105.

In certain embodiments, the VH and VL comprise the amino acid sequences, respectively, set forth in SEQ ID NOs: 67 and 83; 72 and 90; 77 and 96; 66 and 82; 68 and 84; 69 and 85; 68 and 86; 68 and 87; 70 and 88; 71 and 89; 73 and 91; 74 and 92; 75 and 93; 76 and 94; 76 and 95; 76 and 97; 78 and 98; 76 and 99; 79 and 100; 76 and 101; 76 and 102; 76 and 103; 80 and 104; or 81 and 105. In certain embodiments, the VH and VL comprise the amino acid sequences, respectively, set forth in SEQ ID NOs: 67 and 83. In certain embodiments, the VH and VL comprise the amino acid sequences, respectively, set forth in SEQ ID NOs: 72 and 90. In certain embodiments, the VH and VL comprise the amino acid sequences, respectively, set forth in SEQ ID NOs: 77 and 96.

In certain embodiments, the VH and VL are covalently linked. In certain embodiments, the C-terminus of the VH is covalently linked to the N-terminus of the VL, or the C-terminus of the VL is covalently linked to the N-terminus of the VH. In certain embodiments, the VH and VL are covalently linked via a peptide linker. In certain embodiments, the peptide linker is 1-25 amino acids in length. In certain embodiments, the peptide linker comprises an amino acid sequence selected from the group consisting of SEQ ID NOs: 106-118.

In certain embodiments, the antibody is an scFv.

In certain embodiments, the antibody comprises an amino acid sequence selected from the group consisting of SEQ ID NOs: 120-143. In certain embodiments, the antibody consists of an amino acid sequence selected from the group consisting of SEQ ID NOs: 120-143.

In certain embodiments, the antibody is humanized. In certain embodiments, the antibody binds to aggregated TDP-43. In certain embodiments, the antibody binds to phosphorylated TDP-43. In certain embodiments, the antibody binds to non-phosphorylated TDP-43. In certain embodiments, the antibody: reduces levels of aggregated and/or phosphorylated TDP-43 in a cell; increases full-length Stathmin-2 (STMN2) expression, reduces STMN2 cryptic exon (CE) mRNA expression, and/or increases STMN2 protein in neurites as measured with neurite outgrowth, in motor neurons exposed to a cellular stressor; reduces cellular toxicity in response to a cellular stressor; and/or restores motor neuron function as assessed by Microelectrode Array (MEA) system activity.

In an aspect, provided herein is a polypeptide comprising: a VH or VL of an antibody described herein; a VH and VL of an antibody described herein; or an amino acid sequence selected from the group consisting of SEQ ID NOs: 66-105 and 120-143.

In an aspect, provided herein is a polynucleotide comprising a nucleotide sequence encoding an antibody described herein or a polypeptide described herein.

In an aspect, provided herein is a polynucleotide comprising the nucleotide sequence of any one of SEQ ID NOs: 144-215.

In certain embodiments, the polynucleotide further comprises a promoter sequence operably linked to the nucleotide sequence. In certain embodiments, the promoter is selected from the group consisting of a Polymerase II promoter, a chicken-beta actin promoter, a CAG promoter, an EF1alpha promoter, a PGK promoter, and a tissue-specific promoter.

In certain embodiments, the polynucleotide is a vector. In certain embodiments, the vector is a plasmid or a viral vector genome. In certain embodiments, the viral vector genome is a recombinant adeno-associated virus (rAAV) vector genome.

In an aspect, provided herein is an rAAV comprising an AAV capsid and the rAAV vector genome described herein. In certain embodiments, the AAV capsid comprises a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, AAV5.2, or AAV5 capsid protein, or an engineered variant thereof. In certain embodiments, the AAV capsid comprises a capsid protein comprising the amino acid sequence of SEQ ID NO: 216 or 217.

In an aspect, provided herein is a packaging system for preparation of an rAAV, wherein the packaging system comprises: a first nucleotide sequence encoding one or more AAV Rep proteins; a second nucleotide sequence encoding a capsid protein of an rAAV described herein; and a third nucleotide sequence comprising an rAAV genome sequence of an rAAV described herein.

In certain embodiments, the packaging system comprises a first vector comprising the first nucleotide sequence and the second nucleotide sequence, and a second vector comprising the third nucleotide sequence. In certain embodiments, the packaging system further comprises a fourth nucleotide sequence comprising one or more helper virus genes. In certain embodiments, the fourth nucleotide sequence is comprised within a third vector. In certain embodiments, the fourth nucleotide sequence comprises one or more genes from a virus selected from the group consisting of adenovirus, herpes virus, vaccinia virus, and cytomegalovirus (CMV). In certain embodiments, the first vector, second vector, and/or the third vector is a plasmid.

In an aspect, provided herein is a method for recombinant preparation of an rAAV, the method comprising introducing a packaging system described herein into a cell under conditions whereby the rAAV is produced.

In an aspect, provided herein is an antibody described herein, a polynucleotide described herein, or an rAAV described herein, for use in the treatment of a disease or disorder associated with TDP-43 or TDP-43 proteinopathy.

In certain embodiments, the disease or disorder is selected from the group consisting of amyotrophic lateral sclerosis (ALS, including sporadic and familial forms), frontotemporal dementia (FTD, including sporadic and familial forms), Alzheimer's disease (AD, including sporadic and familial forms), Inclusion body myositis (IBM), Oculopharyngeal muscular dystrophy (OPMD), Down syndrome, Familial British dementia, Polyglutamine diseases (including Huntington's disease and spinocerebellar ataxia type 3 (SCA3; also known as Machado Joseph Disease)), Dementia with Lewy Bodies (DLB), or Parkinson's disease (PD). In certain embodiments, the disease or disorder is amyotrophic lateral sclerosis (ALS, including sporadic and familial forms).

In certain embodiments, the treatment comprises delivery of the antibody, polynucleotide, or rAAV to the CNS. In certain embodiments, the antibody, polynucleotide, or rAAV is administered intravenously, intrathecally, intracisternally, intraparenchymally, intravitreally, or subretinally. In certain embodiments, the treatment comprises expression of the antibody in motor neurons in the CNS. In certain embodiments, the treatment comprises a reduction of human TDP-43 protein aggregates in the CNS.

In an aspect, provided herein is a recombinant host cell comprising a polynucleotide described herein or an rAAV described herein.

In an aspect, provided herein is a method of producing an antibody or polypeptide, the method comprising culturing a recombinant host cell described herein under suitable conditions such that the polynucleotide is expressed, and the antibody or polypeptide is produced.

In an aspect, provided herein is a composition comprising an antibody described herein, a polynucleotide described herein, an rAAV described herein, or a host cell described herein, and a pharmaceutically acceptable carrier or excipient.

In an aspect, provided herein is a method of inhibiting an activity of TDP-43 in a subject, the method comprising administering to the subject an effective amount of an antibody or polypeptide described herein, a polynucleotide described herein, an rAAV described herein, a host cell described herein, or a composition described herein.

In an aspect, provided herein is a method of reducing TDP-43 protein aggregates in a subject, the method comprising administering to the subject an effective amount of an antibody or polypeptide described herein, a polynucleotide described herein, an rAAV described herein, a host cell described herein, or a composition described herein.

In an aspect, provided herein is a method of treating a disease or disorder associated with TDP-43 in a subject, the method comprising administering to the subject an effective amount of an antibody or polypeptide described herein, a polynucleotide described herein, an rAAV described herein, a host cell described herein, or a composition described herein.

In certain embodiments, the disease or disorder is selected from the group consisting of Amyotrophic lateral sclerosis (ALS), Frontotemporal dementia (FTD), Alzheimer's disease (AD), Inclusion body myositis (IBM), Oculopharyngeal muscular dystrophy (OPMD), Down syndrome, Familial British dementia, Polyglutamine diseases, Huntington's disease, spinocerebellar ataxia type 3 (SCA3), Dementia with Lewy Bodies (DLB), and Parkinson's disease (PD).

In certain embodiments, the antibody, polypeptide, polynucleotide, rAAV, host cell, or composition is administered intravenously, intrathecally, intracisternally, intraparenchymally, intravitreally, or subretinally.

### BRIEF DESCRIPTION OF THE DRAWINGS

FIG. 1 is a graph showing the percentage of TDP-43 aggregates in the cytoplasm of U2OS human osteosarcoma cells as normalized to control. Cells were transfected with TDP43-ΔNLS1-2KQ-EGFP and either TDP-43 scFv (VecTabs A-I, Y, Z, AA-AM) candidates or a non-TDP-43 scFv control. Graph shows results from three independent experiments in triplicates. Statistical evaluations were calculated by a student's t-test. ns=not significant, *p<0.05, **p<0.01, ***p<0.001.

FIG. 2 is a graph showing the percentage of TDP-43 aggregates in the cytoplasm of U2OS human osteosarcoma cells as normalized to control. Cells were transfected with TDP43-ΔNLS1-2KQ-EGFP and either TDP-43 scFv (VecTabs J-X, AN-BQ) candidates or a non-TDP-43 scFv control. Graph shows results from three independent experiments in triplicates. Statistical evaluations were calculated by a student's t-test. ns=not significant, *p<0.05, **p<0.01, ***p<0.001.

FIG. 3 is a graph showing average intensity for nuclear TDP-43 in stressed M337V motor neurons following treatment with VecTab B, VecTab G, VecTab I, VecTab S, or VecTab U for two weeks at MOI 10\hat{ }6. Intensity was measured by confocal imaging from one experiment (n=7 per group) and normalized to non-treated stressed motor neurons and transduction efficiency. Statistical evaluations were calculated by a one-way ANOVA, ns=not significant, *p<0.1, **p<0.01, ****p<0.0001.

FIG. 4A is a graph showing the change in expression level of full-length (FL) STMN2 in stressed M337V motor neurons, with and without treatment with VecTab B for one week at MOI 10\hat{ }6. Gene expression levels were measured by RT-qPCR and normalized to GAPDH (non-stressed, n=3) and transduction efficiency. Statistical evaluations were calculated by a one-way ANOVA (n=4/treatment, error bars±SEM, ****p<0.0001). FIG. 4B is a graph showing the change in expression level of cryptic exon (CE) STMN2 pathology in stressed M337V motor neurons, with and without treatment with VecTabs B, G, I, S, or U, at an MOI of 10\hat{ }6 for two weeks. Gene expression levels were measured by RT-qPCR and normalized to GAPDH (non-stressed, n=3). Statistical evaluations were calculated by a one-way ANOVA (n=4/treatment, error bars±SEM, ****p<0.0001). FIG. 4C is a graph showing average neurite outgrowth, indicative of STMN2 protein, of stressed M337V motor neurons with and without treatment with VecTab B at an MOI of 10\hat{ }6 for one week and normalized for transduction efficiency. Neurite outgrowth was measured by confocal imaging from one experiment (n=7 per group). Statistical evaluations were calculated by a one-way ANOVA, **p<0.01.

FIG. 5A is a graph showing the average % cell nuclei count of stressed M337V motor neurons when treated with VecTabs B, G, I, S, or U for two weeks, normalized to non-stressed cells treated with a control VecTab (n=6 per group). FIG. 5B is a graph showing the average % cell nuclei count of stressed M337V motor neurons when treated with VecTabs B, G, I, S, or U, normalized to non-stressed, non-treated cells (n=6 per group). Statistical evaluations were calculated by a one-way ANOVA, *<p0.1, **<p0.01.

FIG. 6 is a graph showing the number of neuronal bursts as measured by multielectrode array activity (MEA) in non-stressed M337V motor neurons non-treated (NT) or treated with VecTabs B, G, I, S, or U (n=6 per group) for two weeks, normalized to wild-type cells and transduction efficiency. Statistical evaluations were calculated by a one-way ANOVA, *p<0.1, **p<0.01.

FIG. 7A-FIG. 7D are graphs showing binding of anti-TDP43 clones B, C, G, and I, in full-length human IgG1 format, to non-phosphorylated (FIG. 7A), phosphorylated S409 (FIG. 7B), phosphorylated S410 (FIG. 7C) and phosphorylated S409/S410 (FIG. 7D)C-terminal TDP-43 peptides.

FIG. 8A-FIG. 8D are graphs showing binding of anti-TDP43 clones O, S, U, and X, in full-length human IgG1 format, to non-phosphorylated (FIG. 8A), phosphorylated S409 (FIG. 8B), phosphorylated S410 (FIG. 8C) and phosphorylated S409/S410 (FIG. 8D)C-terminal TDP-43 peptides.

FIG. 9A-FIG. 9D are graphs showing binding of anti-TDP43 clones B, C, G, and I, in human scFv format, to non-phosphorylated (FIG. 9A), phosphorylated S409 (FIG. 9B), phosphorylated S410 (FIG. 9C) and phosphorylated S409/S410 (FIG. 9D)C-terminal TDP-43 peptides.

FIG. 10A-FIG. 10D are graphs showing binding of anti-TDP43 clones O, S, U, and X, in human scFv format, to non-phosphorylated (FIG. 10A), phosphorylated S409 (FIG. 10B), phosphorylated S410 (FIG. 10C) and phosphorylated S409/S410 (FIG. 10D)C-terminal TDP-43 peptides.

### DETAILED DESCRIPTION

The present disclosure provides antibodies and polypeptides that specifically bind to TDP-43 (e.g., human TDP-43). Also provided are pharmaceutical compositions comprising these antibodies, polynucleotides encoding these antibodies, expression vectors and host cells for making these antibodies, and methods of treating a subject using these antibodies. The antibodies disclosed herein are particularly advantageous in that they are able to prevent and/or reduce TDP-43 aggregation, mislocation of TDP-43 from the cell nucleus, and stress-induced neuronal dysfunction.

### Definitions

The expression “TDP-43,” as used herein, refers to transactive response DNA binding protein of 43 kDa, also known as TAR DNA-binding protein 43. The amino acid sequence of full-length TDP-43 can be found at accession number NP_031401.1 (RefSeq). All references to proteins, polypeptides and protein fragments herein are intended to refer to the human version of the respective protein, polypeptide, or protein fragment, unless explicitly specified as being from a non-human species. Thus, the expression “TDP-43” means human TDP-43 unless specified as being from a non-human species, e.g., “mouse TDP-43,” “monkey TDP-43,” etc. The amino acid sequence of human TDP-43 is shown below:

(SEQ ID NO: 119)
MSEYIRVTEDENDEPIEIPSEDDGTVLLSTVTAQFPGACGLRYRNPVSQ
CMRGVRLVEGILHAPDAGWGNLVYVVNYPKDNKRKMDETDASSAVKVKR
AVQKTSDLIVLGLPWKTTEQDLKEYFSTFGEVLMVQVKKDLKTGHSKGF
GFVRFTEYETQVKVMSQRHMIDGRWCDCKLPNSKQSQDEPLRSRKVFVG
RCTEDMTEDELREFFSQYGDVMDVFIPKPFRAFAFVTFADDQIAQSLCG
EDLIIKGISVHISNAEPKHNSNRQLERSGRFGGNPGGFGNQGGFGNSRG
GGAGLGNNQGSNMGGGMNFGAFSINPAMMAAAQAALQSSWGMMGMLASQ
QNQSGPSGNNQNQGNMQREPNQAFGSGNNSYSGSNSGAAIGWGSASNAG
SGSGFNGGFGSSMDSKSSGWGM

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single-chain antibodies or single-chain Fvs (scFv), camelized antibodies, affibodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG1, IgG2, IgG3, IgG4, IgA1, or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In a specific embodiment, the antibody is a humanized monoclonal antibody. In another specific embodiment, the antibody is a human monoclonal antibody.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of protein of immunological interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other. In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human TDP-43). CDRH1, CDRH2, and CDRH3 denote the heavy chain CDRs, and CDRL1, CDRL2, and CDRL3 denote the light chain CDRs.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In certain embodiments, the variable region is a primate (e.g., non-human primate) variable region. In certain embodiments, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ), based on the amino acid sequence of the constant region. Light chain amino acid sequences are well known in the art. In specific embodiments, the light chain is a human light chain.

As used herein, the terms “specifically binds,” “specifically recognizes,” “immunospecifically binds,” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, Biacore™, KinExA® 3000 instrument (Sapidyne Instruments, Boise, ID), or other assays known in the art. In a specific embodiment, molecules that specifically bind to an antigen bind to the antigen with a K_{A} that is at least 2 logs (e.g., factors of 10), 2.5 logs, 3 logs, 4 logs, or greater than the K_{A} when the molecules bind non-specifically to another antigen.

As used herein, the term “affinity” refers to the strength of the total sum of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety.

As used herein, the term “AAV” is a standard abbreviation for adeno-associated virus.

As used herein, the term “recombinant adeno-associated virus” or “rAAV” refers to an AAV comprising a genome lacking functional rep and cap genes.

As used herein, the term “cap gene” refers to a nucleotide sequence that encodes a capsid protein (“Cap”). For AAV, the capsid protein may be VP1, VP2, or VP3. VP1, VP2, and/or VP3 capsid proteins assemble into a capsid that surrounds the rAAV genome.

As used herein, the term “rep gene” refers to the nucleotide sequences that encode the non-structural proteins (e.g., rep78, rep68, rep52, and rep40) required for the replication and production of an AAV (“Rep”).

As used herein, the term “rAAV genome” refers to a polynucleotide (e.g., DNA and/or RNA) comprising the genome sequence of an rAAV. The skilled artisan will appreciate that where an rAAV genome comprises a transgene (e.g., an antibody), the rAAV genome can be in the sense or antisense orientation relative to the direction of transcription of the transgene.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In certain embodiments, the subject is a human or non-human mammal. In certain embodiments, the subject is a human.

As used herein with respect to an antibody or polynucleotide, the term “isolated” refers to an antibody or polynucleotide that is separated from one or more contaminants (e.g., polypeptides, polynucleotides, lipids, or carbohydrates, etc.) which are present in a natural source of the antibody or polynucleotide. All instances of “isolated antibodies” described herein are additionally contemplated as antibodies that may be, but need not be, isolated. All instances of “isolated polynucleotides” described herein are additionally contemplated as polynucleotides that may be, but need not be, isolated. All instances of “antibodies” described herein are additionally contemplated as antibodies that may be, but need not be, isolated. All instances of “polynucleotides” described herein are additionally contemplated as polynucleotides that may be, but need not be, isolated.

As used herein, the term “about” or “approximately” when referring to a measurable value, such as a dosage, encompasses variations of ±20% or ±10%, ±5%, ±1%, or ±0.1% of a given value or range, as are appropriate to perform the methods disclosed herein.

The determination of “percent identity” between two sequences (e.g., amino acid sequences or nucleotide sequences) can be accomplished using a mathematical algorithm. A specific, non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a polynucleotide described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### Anti-TDP-43 Antibodies

In one aspect, the instant disclosure provides antibodies that specifically bind to TDP-43 (e.g., human TDP-43). The amino acid sequences of exemplary antibodies are set forth in Table 1 below.

TABLE 1
Amino acid sequences of exemplary anti-TDP-43 antibodies.

| | | SEQ ID | |
|---|---|---|---|
| Clone | Description | NO: | Amino Acid Sequence |
| A | CDRH1 | 1 | GKYIH |
| | CDRH2 | 2 | GINPNNGGTSYNQKFQG |
| | CDRH3 | | KS |
| | CDRL1 | 3 | RSSKSLLHSNGNTYLY |
| | CDRL2 | 4 | RMSNLAS |
| | CDRL3 | 5 | MQGLQTPLT |
| | VH | 66 | QVQLVQSGAEVKKPGASVKVSCKASGYAFTGKYIHWVR |
| | | | QAPGQGLEWMGGINPNNGGTSYNQKFQGRVTMTRDTST |
| | | | STVYMELSSLRSEDTAVYYCARKSWGQGTLVTVSS |
| | VL | 82 | DIVMTQSPLSLPVTPGEPASISCRSSKSLLHSNGNTYLYWY |
| | | | LQKPGQSPQLLIYRMSNLASGVPDRFSGSGSGTDFTLKISR |
| | | | VEAEDVGVYYCMQGLQTPLTFGGGTKVEIK |
| B | CDRH1 | 6 | ELSMH |
| | CDRH2 | 2 | GINPNNGGTSYNQKFQG |
| | CDRH3 | | ES |
| | CDRL1 | 7 | KSSQSLLHSDGKTYLN |
| | CDRL2 | 8 | LASRRAS |
| | CDRL3 | 9 | FQGTHFPHT |
| | VH | 67 | QVQLVQSGAEVKKPGASVKVSCKASGFTFTELSMHWVR |
| | | | QAPGQGLEWMGGINPNNGGTSYNQKFQGRVTMTRDTST |
| | | | STVYMELSSLRSEDTAVYYCARESWGQGTLVTVSS |
| | VL | 83 | DIVMTQSPLSLPVTPGEPASISCKSSQSLLHSDGKTYLNWY |
| | | | LQKPGQSPQLLIYLASRRASGVPDRFSGSGSGTDFTLKISR |
| | | | VEAEDVGVYYCFQGTHFPHTFGGGTKVEIK |
| C | CDRH1 | 10 | EYSMH |
| | CDRH2 | 2 | GINPNNGGTSYNQKFQG |
| | CDRH3 | | ES |
| | CDRL1 | 7 | KSSQSLLHSDGKTYLN |
| | CDRL2 | 11 | LGSNRAS |
| | CDRL3 | 9 | FQGTHFPHT |
| | VH | 68 | QVQLVQSGAEVKKPGASVKVSCKASGFTFTEYSMHWVR |
| | | | QAPGQGLEWMGGINPNNGGTSYNQKFQGRVTMTRDTST |
| | | | STVYMELSSLRSEDTAVYYCARESWGQGTLVTVSS |
| | VL | 84 | DIVMTQSPLSLPVTPGEPASISCKSSQSLLHSDGKTYLNWY |
| | | | LQKPGQSPQLLIYLGSNRASGVPDRFSGSGSGTDFTLKISR |
| | | | VEAEDVGVYYCFQGTHFPHTFGGGTKVEIK |
| D | CDRH1 | 10 | EYSMH |
| | CDRH2 | 12 | GINPNNGGTRYNQKFQG |
| | CDRH3 | | ES |
| | CDRL1 | 7 | KSSQSLLHSDGKTYLN |
| | CDRL2 | 13 | LVSNRAS |
| | CDRL3 | 14 | HQGTHFPHT |
| | VH | 69 | QVQLVQSGAEVKKPGASVKVSCKASGGTFTEYSMHWVR |
| | | | QAPGQGLEWMGGINPNNGGTRYNQKFQGRVTMTRDTST |
| | | | STVYMELSSLRSEDTAVYYCARESWGQGTLVTVSS |
| | VL | 85 | DIVMTQSPLSLPVTPGEPASISCKSSQSLLHSDGKTYLNWY |
| | | | LQKPGQSPQLLIYLVSNRASGVPDRFSGSGSGTDFTLKISR |
| | | | VEAEDVGVYYCHQGTHFPHTFGGGTKVEIK |
| E | CDRH1 | 10 | EYSMH |
| | CDRH2 | 2 | GINPNNGGTSYNQKFQG |
| | CDRH3 | | ES |
| | CDRL1 | 7 | KSSQSLLHSDGKTYLN |
| | CDRL2 | 15 | LGSSRAS |
| | CDRL3 | 16 | MQGTHFPHT |
| | VH | 68 | QVQLVQSGAEVKKPGASVKVSCKASGFTFTEYSMHWVR |
| | | | QAPGQGLEWMGGINPNNGGTSYNQKFQGRVTMTRDTST |
| | | | STVYMELSSLRSEDTAVYYCARESWGQGTLVTVSS |
| | VL | 86 | DIVMTQSPLSLPVTPGEPASISCKSSQSLLHSDGKTYLNWY |
| | | | LQKPGQSPQLLIYLGSSRASGVPDRFSGSGSGTDFTLKISR |
| | | | VEAEDVGVYYCMQGTHFPHTFGGGTKVEIK |
| F | CDRH1 | 10 | EYSMH |
| | CDRH2 | 2 | GINPNNGGTSYNQKFQG |
| | CDRH3 | | ES |
| | CDRL1 | 17 | KSSQSLLHNDGKTYLN |
| | CDRL2 | 18 | LASNRAS |
| | CDRL3 | 19 | GQGTHFPHT |
| | VH | 68 | QVQLVQSGAEVKKPGASVKVSCKASGFTFTEYSMHWVR |
| | | | QAPGQGLEWMGGINPNNGGTSYNQKFQGRVTMTRDTST |
| | | | STVYMELSSLRSEDTAVYYCARESWGQGTLVTVSS |
| | VL | 87 | DIVMTQSPLSLPVTPGEPASISCKSSQSLLHNDGKTYLNWY |
| | | | LQKPGQSPQLLIYLASNRASGVPDRFSGSGSGTDFTLKISR |
| | | | VEAEDVGVYYCGQGTHFPHTFGGGTKVEIK |
| G | CDRH1 | 10 | EYSMH |
| | CDRH2 | 2 | GINPNNGGTSYNQKFQG |
| | CDRH3 | | ES |
| | CDRL1 | 7 | KSSQSLLHSDGKTYLN |
| | CDRL2 | 20 | LVSKLKS |
| | CDRL3 | 16 | MQGTHFPHT |
| | VH | 70 | QVQLVQSGAEVKKPGASVKVSCKASGFTFTEYSMHWVR |
| | | | QAPGQGLEWMGGINPNNGGTSYNQKFQGRVTMTRDTST |
| | | | STVYMELSSLRSEDTAVYYCASESWGQGTLVTVSS |
| | VL | 88 | DIVMTQSPLSLPVTPGEPASISCKSSQSLLHSDGKTYLNWY |
| | | | LQKPGQSPQLLIYLVSKLKSGVPDRFSGSGSGTDFTLKISR |
| | | | VEAEDVGVYYCMQGTHFPHTFGGGTKVEIK |
| H | CDRH1 | 10 | EYSMH |
| | CDRH2 | 12 | GINPNNGGTRYNQKFQG |
| | CDRH3 | | ES |
| | CDRL1 | 21 | KSSQSLLHKDGKTYLN |
| | CDRL2 | 22 | LVSKLPS |
| | CDRL3 | 16 | MQGTHFPHT |
| | VH | 71 | QVQLVQSGAEVKKPGASVKVSCKASGFTFTEYSMHWVR |
| | | | QAPGQGLEWMGGINPNNGGTRYNQKFQGRVTMTRDTST |
| | | | STVYMELSSLRSEDTAVYYCARESWGQGTLVTVSS |
| | VL | 89 | DIVMTQSPLSLPVTPGEPASISCKSSQSLLHKDGKTYLNWY |
| | | | LQKPGQSPQLLIYLVSKLPSGVPDRFSGSGSGTDFTLKISRV |
| | | | EAEDVGVYYCMQGTHFPHTFGGGTKVEIK |
| I | CDRH1 | 10 | EYSMH |
| | CDRH2 | 23 | GINPNNGGTIYNQKFQG |
| | CDRH3 | | ES |
| | CDRL1 | 7 | KSSQSLLHSDGKTYLN |
| | CDRL2 | 11 | LGSNRAS |
| | CDRL3 | 16 | MQGTHFPHT |
| | VH | 72 | QVQLVQSGAEVKKPGASVKVSCKASGFTFTEYSMHWVR |
| | | | QAPGQGLEWMGGINPNNGGTIYNQKFQGRVTMTRDTSTS |
| | | | TVYMELSSLRSEDTAVYYCARESWGQGTLVTVSS |
| | VL | 90 | DIVMTQSPLSLPVTPGEPASISCKSSQSLLHSDGKTYLNWY |
| | | | LQKPGQSPQLLIYLGSNRASGVPDRFSGSGSGTDFTLKISR |
| | | | VEAEDVGVYYCMQGTHFPHTFGGGTKVEIK |
| J | CDRH1 | 24 | SYAIS |
| | CDRH2 | 25 | EINPSNGRTVYNQKFQG |
| | CDRH3 | 26 | YKDY |
| | CDRL1 | 27 | RSSQSIVTAIGNTYLE |
| | CDRL2 | 28 | KVSNRFS |
| | CDRL3 | 29 | FQGSHVPFT |
| | VH | 73 | QVQLVQSGAEVKKPGASVKVSCKASGGTFSSYAISWVRQ |
| | | | APGQGLEWMGEINPSNGRTVYNQKFQGRVTMTRDTSTST |
| | | | VYMELSSLRSEDTAVYYCARYKDYWGQGTLVTVSS |
| | VL | 91 | DIVMTQSPLSLPVTPGEPASISCRSSQSIVTAIGNTYLEWYL |
| | | | QKPGQSPQLLIYKVSNRFSGVPDRFSGSGSGTDFTLKISRV |
| | | | EAEDVGVYYCFQGSHVPFTFGGGTKVEIK |
| K | CDRH1 | 30 | SYAIH |
| | CDRH2 | 31 | EINPSNGRTNYNQKFQG |
| | CDRH3 | 32 | RMDY |
| | CDRL1 | 3 | RSSKSLLHSNGNTYLY |
| | CDRL2 | 33 | RMSNRAS |
| | CDRL3 | 34 | MQALQTPFT |
| | VH | 74 | QVQLVQSGAEVKKPGASVKVSCKASGGTFSSYAIHWVRQ |
| | | | APGQGLEWMGEINPSNGRTNYNQKFQGRVTMTRDTSTST |
| | | | VYMELSSLRSEDTAVYYCARRMDYWGQGTLVTVSS |
| | VL | 92 | DIVMTQSPLSLPVTPGEPASISCRSSKSLLHSNGNTYLYWY |
| | | | LQKPGQSPQLLIYRMSNRASGVPDRFSGSGSGTDFTLKISR |
| | | | VEAEDVGVYYCMQALQTPFTFGGGTKVEIK |
| L | CDRH1 | 35 | KYWMH |
| | CDRH2 | 36 | SMNPNSGHTGFAQKFQG |
| | CDRH3 | 37 | AMDY |
| | CDRL1 | 38 | KSSQSLLHSDGKTYLY |
| | CDRL2 | 39 | KVSNRFK |
| | CDRL3 | 40 | MQKIQLPVT |
| | VH | 75 | QVQLVQSGAEVKKPGASVKVSCKASGYTFTKYWMHWV |
| | | | RQAPGQGLEWVGSMNPNSGHTGFAQKFQGRVTMTRDTS |
| | | | TSTVYMELSSLRSEDTAVYYCARAMDYWGQGTLVTVSS |
| | VL | 93 | DIVMTQSPLSLPVTPGEPASISCKSSQSLLHSDGKTYLYWY |
| | | | LQKPGQSPQLLIYKVSNRFKGVPDRFSGSGSGTDFTLKISR |
| | | | VEAEDVGVYYCMQKIQLPVTFGGGTKVEIK |
| M | CDRH1 | 35 | KYWMH |
| | CDRH2 | 31 | EINPSNGRTNYNQKFQG |
| | CDRH3 | 41 | YMDY |
| | CDRL1 | 42 | RSSESIVHSNGNTYLE |
| | CDRL2 | 43 | KVSNKFS |
| | CDRL3 | 44 | MQYTHWPPT |
| | VH | 76 | QVQLVQSGAEVKKPGASVKVSCKASGYTFTKYWMHWV |
| | | | RQAPGQGLEWMGEINPSNGRTNYNQKFQGRVTMTRDTST |
| | | | STVYMELSSLRSEDTAVYYCWRYMDYWGQGTLVTVSS |
| | VL | 94 | DIVMTQSPLSLPVTPGEPASISCRSSESIVHSNGNTYLEWYL |
| | | | QKPGQSPQLLIYKVSNKFSGVPDRFSGSGSGTDFTLKISRV |
| | | | EAEDVGVYYCMQYTHWPPTFGGGTKVEIK |
| N | CDRH1 | 35 | KYWMH |
| | CDRH2 | 31 | EINPSNGRTNYNQKFQG |
| | CDRH3 | 41 | YMDY |
| | CDRL1 | 45 | RSSQSLLHSNGYNYLD |
| | CDRL2 | 46 | KVENRFS |
| | CDRL3 | 47 | MQHTHWPPT |
| | VH | 76 | QVQLVQSGAEVKKPGASVKVSCKASGYTFTKYWMHWV |
| | | | RQAPGQGLEWMGEINPSNGRTNYNQKFQGRVTMTRDTST |
| | | | STVYMELSSLRSEDTAVYYCWRYMDYWGQGTLVTVSS |
| | VL | 95 | DIVMTQSPLSLPVTPGEPASISCRSSQSLLHSNGYNYLDWY |
| | | | LQKPGQSPQLLIYKVENRFSGVPDRFSGSGSGTDFTLKISR |
| | | | VEAEDVGVYYCMQHTHWPPTFGGGTKVEIK |
| O | CDRH1 | 35 | KYWMH |
| | CDRH2 | 48 | EINPKNGRTNYNQKFQG |
| | CDRH3 | 41 | YMDY |
| | CDRL1 | 45 | RSSQSLLHSNGYNYLD |
| | CDRL2 | 28 | KVSNRFS |
| | CDRL3 | 49 | LQHTHWPPT |
| | VH | 77 | QVQLVQSGAEVKKPGASVKVSCKASGYTFTKYWMHWV |
| | | | RQAPGQGLEWMGEINPKNGRTNYNQKFQGRVTMTRDTS |
| | | | TSTVYMELSSLRSEDTAVYYCWRYMDYWGQGTLVTVSS |
| | VL | 96 | DIVMTQSPLSLPVTPGEPASISCRSSQSLLHSNGYNYLDWY |
| | | | LQKPGQSPQLLIYKVSNRFSGVPDRFSGSGSGTDFTLKISR |
| | | | VEAEDVGVYYCLQHTHWPPTFGGGTKVEIK |
| P | CDRH1 | 35 | KYWMH |
| | CDRH2 | 31 | EINPSNGRTNYNQKFQG |
| | CDRH3 | 41 | YMDY |
| | CDRL1 | 45 | RSSQSLLHSNGYNYLD |
| | CDRL2 | 50 | EVSNRFS |
| | CDRL3 | 47 | MQHTHWPPT |
| | VH | 76 | QVQLVQSGAEVKKPGASVKVSCKASGYTFTKYWMHWV |
| | | | RQAPGQGLEWMGEINPSNGRTNYNQKFQGRVTMTRDTST |
| | | | STVYMELSSLRSEDTAVYYCWRYMDYWGQGTLVTVSS |
| | VL | 97 | DIVMTQSPLSLPVTPGEPASISCRSSQSLLHSNGYNYLDWY |
| | | | LQKPGQSPQLLIYEVSNRFSGVPDRFSGSGSGTDFTLKISR |
| | | | VEAEDVGVYYCMQHTHWPPTFGGGTKVEIK |
| Q | CDRH1 | 35 | KYWMH |
| | CDRH2 | 51 | EINPQNGRTNYNQKFQG |
| | CDRH3 | 41 | YMDY |
| | CDRL1 | 52 | RSSRSLLHSNGYNYLD |
| | CDRL2 | 53 | AASSLQS |
| | CDRL3 | 49 | LQHTHWPPT |
| | VH | 78 | QVQLVQSGAEVKKPGASVKVSCKASGYTFTKYWMHWV |
| | | | RQAPGQGLEWMGEINPQNGRTNYNQKFQGRVTMTRDTS |
| | | | TSTVYMELSSLRSEDTAVYYCWRYMDYWGQGTLVTVSS |
| | VL | 98 | DIVMTQSPLSLPVTPGEPASISCRSSRSLLHSNGYNYLDWY |
| | | | LQKPGQSPQLLIYAASSLQSGVPDRFSGSGSGTDFTLKISR |
| | | | VEAEDVGVYYCLQHTHWPPTFGGGTKVEIK |
| R | CDRH1 | 35 | KYWMH |
| | CDRH2 | 31 | EINPSNGRTNYNQKFQG |
| | CDRH3 | 41 | YMDY |
| | CDRL1 | 45 | RSSQSLLHSNGYNYLD |
| | CDRL2 | 28 | KVSNRFS |
| | CDRL3 | 47 | MQHTHWPPT |
| | VH | 76 | QVQLVQSGAEVKKPGASVKVSCKASGYTFTKYWMHWV |
| | | | RQAPGQGLEWMGEINPSNGRTNYNQKFQGRVTMTRDTST |
| | | | STVYMELSSLRSEDTAVYYCWRYMDYWGQGTLVTVSS |
| | VL | 99 | DIVMTQSPLSLPVTPGEPASISCRSSQSLLHSNGYNYLDWY |
| | | | LQKPGQSPQLLIYKVSNRFSGVPDRFSGSGSGTDFTLKISR |
| | | | VEAEDVGVYYCMQHTHWPPTFGGGTKVEIK |
| S | CDRH1 | 35 | KYWMH |
| | CDRH2 | 54 | EINPSNGRTNYPQKFQG |
| | CDRH3 | 41 | YMDY |
| | CDRL1 | 45 | RSSQSLLHSNGYNYLD |
| | CDRL2 | 55 | KVSNRFT |
| | CDRL3 | 56 | MQHTHWPPA |
| | VH | 79 | QVQLVQSGAEVKKPGASVKVSCKASGYTFTKYWMHWV |
| | | | RQAPGQGLEWMGEINPSNGRTNYPQKFQGRVTMTRDTST |
| | | | STVYMELSSLRSEDTAVYYCWRYMDYWGQGTLVTVSS |
| | VL | 100 | DIVMTQSPLSLPVTPGEPASISCRSSQSLLHSNGYNYLDWY |
| | | | LQKPGQSPQLLIYKVSNRFTGVPDRFSGSGSGTDFTLKISR |
| | | | VEAEDVGVYYCMQHTHWPPAFGGGTKVEIK |
| T | CDRH1 | 35 | KYWMH |
| | CDRH2 | 31 | EINPSNGRTNYNQKFQG |
| | CDRH3 | 41 | YMDY |
| | CDRL1 | 45 | RSSQSLLHSNGYNYLD |
| | CDRL2 | 57 | KSSNRFS |
| | CDRL3 | 47 | MQHTHWPPT |
| | VH | 76 | QVQLVQSGAEVKKPGASVKVSCKASGYTFTKYWMHWV |
| | | | RQAPGQGLEWMGEINPSNGRTNYNQKFQGRVTMTRDTST |
| | | | STVYMELSSLRSEDTAVYYCWRYMDYWGQGTLVTVSS |
| | VL | 101 | DIVMTQSPLSLPVTPGEPASISCRSSQSLLHSNGYNYLDWY |
| | | | LQKPGQSPQLLIYKSSNRFSGVPDRFSGSGSGTDFTLKISRV |
| | | | EAEDVGVYYCMQHTHWPPTFGGGTKVEIK |
| U | CDRH1 | 35 | KYWMH |
| | CDRH2 | 31 | EINPSNGRTNYNQKFQG |
| | CDRH3 | 41 | YMDY |
| | CDRL1 | 58 | RSSQSLLNSNGVTFVE |
| | CDRL2 | 50 | EVSNRFS |
| | CDRL3 | 49 | LQHTHWPPT |
| | VH | 76 | QVQLVQSGAEVKKPGASVKVSCKASGYTFTKYWMHWV |
| | | | RQAPGQGLEWMGEINPSNGRTNYNQKFQGRVTMTRDTST |
| | | | STVYMELSSLRSEDTAVYYCWRYMDYWGQGTLVTVSS |
| | VL | 102 | DIVMTQSPLSLPVTPGEPASISCRSSQSLLNSNGVTFVEWY |
| | | | LQKPGQSPQLLIYEVSNRFSGVPDRFSGSGSGTDFTLKISR |
| | | | VEAEDVGVYYCLQHTHWPPTFGGGTKVEIK |
| V | CDRH1 | 35 | KYWMH |
| | CDRH2 | 31 | EINPSNGRTNYNQKFQG |
| | CDRH3 | 41 | YMDY |
| | CDRL1 | 45 | RSSQSLLHSNGYNYLD |
| | CDRL2 | 59 | EVSNRFW |
| | CDRL3 | 49 | LQHTHWPPT |
| | VH | 76 | QVQLVQSGAEVKKPGASVKVSCKASGYTFTKYWMHWV |
| | | | RQAPGQGLEWMGEINPSNGRTNYNQKFQGRVTMTRDTST |
| | | | STVYMELSSLRSEDTAVYYCWRYMDYWGQGTLVTVSS |
| | VL | 103 | DIVMTQSPLSLPVTPGEPASISCRSSQSLLHSNGYNYLDWY |
| | | | LQKPGQSPQLLIYEVSNRFWGVPDRFSGSGSGTDFTLKISR |
| | | | VEAEDVGVYYCLQHTHWPPTFGGGTKVEIK |
| W | CDRH1 | 35 | KYWMH |
| | CDRH2 | 60 | GINPSNGRTNYNQKFQG |
| | CDRH3 | 61 | YMQY |
| | CDRL1 | 3 | RSSKSLLHSNGNTYLY |
| | CDRL2 | 62 | KVSNQSS |
| | CDRL3 | 63 | MQALQTPLT |
| | VH | 80 | QVQLVQSGAEVKKPGASVKVSCKASGYTFTKYWMHWV |
| | | | RQAPGQGLEWMGGINPSNGRTNYNQKFQGRVTMTRDTS |
| | | | TSTVYMELSSLRSEDTAVYYCARYMQYWGQGTLVTVSS |
| | VL | 104 | DIVMTQSPLSLPVTPGEPASISCRSSKSLLHSNGNTYLYWY |
| | | | LQKPGQSPQLLIYKVSNQSSGVPDRFSGSGSGTDFTLKISR |
| | | | VEAEDVGVYYCMQALQTPLTFGGGTKVEIK |
| X | CDRH1 | 64 | SHYLH |
| | CDRH2 | 65 | IINPSNGRTNYNQKFQG |
| | CDRH3 | 41 | YMDY |
| | CDRL1 | 45 | RSSQSLLHSNGYNYLD |
| | CDRL2 | 50 | EVSNRFS |
| | CDRL3 | 49 | LQHTHWPPT |
| | VH | 81 | QVQLVQSGAEVKKPGASVKVSCKASGYTFTSHYLHWVR |
| | | | QAPGQGLEWMGIINPSNGRTNYNQKFQGRVTMTRDTSTS |
| | | | TVYMELSSLRSEDTAVYYCWRYMDYWGQGTLVTVSS |
| | VL | 105 | DIVMTQSPLSLPVTPGEPASISCRSSQSLLHSNGYNYLDWY |
| | | | LQKPGQSPQLLIYEVSNRFSGVPDRFSGSGSGTDFTLKISR |
| | | | VEAEDVGVYYCLQHTHWPPTFGGGTKVEIK |

In certain embodiments, the instant disclosure provides an antibody that specifically binds to TDP-43 (e.g., human TDP-43), the antibody comprising a VH domain comprising one, two, or all three of the CDRs of a VH domain set forth in Table 1. In certain embodiments, the antibody comprises the CDRH1 of a VH domain set forth in Table 1. In certain embodiments, the antibody comprises the CDRH2 of a VH domain set forth in Table 1. In certain embodiments, the antibody comprises the CDRH3 of a VH domain set forth in Table 1.

In certain embodiments, the instant disclosure provides an antibody that specifically binds to TDP-43 (e.g., human TDP-43), the antibody comprising a VL domain comprising one, two, or all three of the CDRs of a VL domain disclosed in Table 1. In certain embodiments, the antibody comprises the CDRL1 of a VL domain set forth in Table 1. In certain embodiments, the antibody comprises the CDRL2 of a VL domain set forth in Table 1. In certain embodiments, the antibody comprises the CDRL3 of a VL domain set forth in Table 1.

The individual CDRs of an antibody disclosed herein can be determined according to any CDR numbering scheme known in the art.

In certain embodiments, one or more of the CDRs of an antibody disclosed herein can be determined according to Kabat et al., J. Biol. Chem. 252, 6609-6616 (1977) and Kabat et al., Sequences of protein of immunological interest (1991), each of which is herein incorporated by reference in its entirety.

In certain embodiments, the instant disclosure provides antibodies that specifically bind to TDP-43 (e.g., human TDP-43) and comprise CDRs of an antibody disclosed in Table 1 herein as determined by the Kabat numbering scheme.

In certain embodiments, one or more of the CDRs of an antibody disclosed herein can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226, all of which are herein incorporated by reference in their entireties).

In certain embodiments, the instant disclosure provides antibodies that specifically bind to TDP-43 (e.g., human TDP-43) and comprise CDRs of an antibody disclosed in Table 1 herein, as determined by the Chothia numbering system.

In certain embodiments, one or more of the CDRs of an antibody disclosed herein can be determined according to MacCallum R M et al., (1996) J Mol Biol 262:732-745, herein incorporated by reference in its entirety. See, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001), herein incorporated by reference in its entirety.

In certain embodiments, the instant disclosure provides antibodies that specifically bind to TDP-43 (e.g., human TDP-43) and comprise CDRs of an antibody disclosed in Table 1 herein, as determined by the MacCallum numbering system.

In certain embodiments, the CDRs of an antibody disclosed herein can be determined according to the IMGT numbering system as described in: Lefranc M-P, (1999) The Immunologist 7:132-136; Lefranc M-P et al., (1999) Nucleic Acids Res 27:209-212, each of which is herein incorporated by reference in its entirety; and Lefranc M-P et al., (2009) Nucleic Acids Res 37: D1006-D1012.

In certain embodiments, the instant disclosure provides antibodies that specifically bind to TDP-43 (e.g., human TDP-43) and comprise CDRs of an antibody disclosed in Table 1 herein, as determined by the IMGT numbering system.

In certain embodiments, the CDRs of an antibody disclosed herein can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.), herein incorporated by reference in its entirety.

In certain embodiments, the instant disclosure provides antibodies that specifically bind to TDP-43 (e.g., human TDP-43) and comprise CDRs of an antibody disclosed in Table 1 herein as determined by the AbM numbering scheme.

In certain embodiments, the CDRs of an antibody disclosed herein can be determined according to the AHo numbering system, as described in Honegger and Plückthun, A., J. Mol. Biol. 309:657-670 (2001), herein incorporated by reference in its entirety.

In certain embodiments, the instant disclosure provides antibodies that specifically bind to TDP-43 (e.g., human TDP-43) and comprise CDRs of an antibody disclosed in Table 1 herein, as determined by the AHo numbering system.

In certain embodiments, the individual CDRs of an antibody disclosed herein are each independently determined according to one of the Kabat, Chothia, MacCallum, IMGT, AHo, or AbM numbering schemes, or by structural analysis of the multispecific molecule, wherein the structural analysis identifies residues in the variable region(s) predicted to make contact with an epitope region of TDP-43.

In certain embodiments, the instant disclosure provides an antibody that specifically bind TDP-43 (e.g., human TDP-43) comprising a VH comprising the CDRH1, CDRH2, and CDRH3 amino acid sequences of any one of the VH amino acid sequences set forth in SEQ ID NOs: 66-81, and a VL comprising the CDRL1, CDRL2, and CDRL3 amino acid sequences of any one of the VL amino acid sequences set forth in SEQ ID NOs: 82-105, wherein each CDR is independently determined according to one of the Kabat, Chothia, MacCallum, IMGT, AHo, or AbM numbering schemes, or by structural analysis of the multispecific molecule, wherein the structural analysis identifies residues in the variable region(s) predicted to make contact with an epitope region of TDP-43 (e.g., human TDP-43).

In certain embodiments, the instant disclosure provides an antibody that specifically binds to TDP-43 (e.g., human TDP-43), wherein the antibody comprises the CDRH1, CDRH2, CDRH3, CDRL1, CDRL2, and CDRL3 amino acid sequences of the VH and VL amino acid sequences set forth in SEQ ID NOs: 66 and 82; 67 and 83; 68 and 84; 69 and 85; 68 and 86; 68 and 87; 70 and 88; 71 and 89; 72 and 90; 73 and 91; 74 and 92; 75 and 93; 76 and 94; 76 and 95; 77 and 96; 76 and 97; 78 and 98; 76 and 99; 79 and 100; 76 and 101; 76 and 102; 76 and 103; 80 and 104; or 81 and 105, respectively.

In certain embodiments, the instant disclosure provides an antibody that specifically binds to TDP-43 (e.g., human TDP-43), wherein the antibody comprises a VH comprising CDRH1, CDRH2, and CDRH3 regions, and a VL comprising CDRL1, CDRL2, and CDRL3 regions, wherein the CDRH1, CDRH2, CDRH3, CDRL1, CDRL2, and CDRL3 regions comprise the amino acid sequences set forth in SEQ ID NOs: 6, 2, ES, 7, 8 and 9; 10, 23, ES, 7, 11 and 16; 35, 48, 41, 45, 28 and 49; 1, 2, KS, 3, 4 and 5; 10, 2, ES, 7, 11 and 9; 10, 12, ES, 7, 13 and 14; 10, 2, ES, 7, 15 and 16; 10, 2, ES, 17, 18 and 19; 10, 2, ES, 7, 20 and 16; 10, 12, ES, 21, 22 and 16; 24, 25, 26, 27, 28 and 29; 30, 31, 32, 3, 33 and 34; 35, 36, 37, 38, 39 and 40; 35, 31, 41, 42, 43 and 44; 35, 31, 41, 45, 46 and 47; 35, 31, 41, 45, 50 and 47; 35, 51, 41, 52, 53 and 49; 35, 31, 41, 45, 28 and 47; 35, 54, 41, 45, 55 and 56; 35, 31, 41, 45, 57 and 47; 35, 31, 41, 58, 50 and 49; 35, 31, 41, 45, 59 and 49; 35, 60, 61, 3, 62 and 63; or 64, 65, 41, 45, 50 and 49, respectively.

In certain embodiments, the instant disclosure provides an antibody that specifically binds to TDP-43 (e.g., human TDP-43), wherein the antibody comprises a VH comprising CDRH1, CDRH2, and CDRH3 regions, and a VL comprising CDRL1, CDRL2, and CDRL3 regions, wherein the CDRH1, CDRH2, CDRH3, CDRL1, CDRL2, and CDRL3 regions comprise the amino acid sequences set forth in SEQ ID NOs: 6, 2, ES, 7, 8, and 9, respectively. In certain embodiments, the CDRH1, CDRH2, CDRH3, CDRL1, CDRL2, and CDRL3 regions comprise the amino acid sequences set forth in SEQ ID NOs: 10, 23, ES, 7, 11 and 16, respectively. In certain embodiments, the CDRH1, CDRH2, CDRH3, CDRL1, CDRL2, and CDRL3 regions comprise the amino acid sequences set forth in SEQ ID NOs: 35, 48, 41, 45, 28 and 49, respectively.

In certain embodiments, the instant disclosure provides an antibody that specifically binds to TDP-43 (e.g., human TDP-43), wherein the antibody comprises framework regions (e.g., framework regions of the VL domain and/or VH domain) that are human framework regions or derived from human framework regions. Non-limiting examples of human framework regions are described in the art, e.g., see Kabat E A et al., (1991) supra). In certain embodiments, the antibody comprises the VH framework regions of an antibody set forth in Table 1. In certain embodiments, the antibody comprises the VL framework regions (FRs) of an antibody set forth in Table 1.

In certain embodiments, the VH and VL comprise human or human-derived framework regions. In certain embodiments, the VH comprises IGHV1-46 framework sequences, or framework sequences having at least 70%, at least 75%, at least 80%, at least 85%, at least 90%, at least 95%, or at least 98% sequence identity to IGHV1-46 framework sequences. In certain embodiments, the VL comprises IGKV2-28, IGKV2D-28, IGKV2-29, IGKV2D-29, IGKV2-40, or IGKV2D-40 framework sequences, or framework sequences having at least 70%, at least 75%, at least 80%, at least 85%, at least 90%, at least 95%, or at least 98% sequence identity to IGKV2-28, IGKV2D-28, IGKV2-29, IGKV2D-29, IGKV2-40, or IGKV2D-40 framework sequences.

In certain embodiments, the instant disclosure provides an antibody that specifically binds to TDP-43 (e.g., human TDP-43), comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to an amino acid sequence set forth in any one of SEQ ID NOs: 66-81. In certain embodiments, the instant disclosure provides an antibody that specifically binds to TDP-43 (e.g., human TDP-43), comprising a VH comprising an amino acid sequence set forth in any one of SEQ ID NOs: 66-81. In certain embodiments, the amino acid sequence of the VH consists of an amino acid sequence set forth in any one of SEQ ID NOs: 66-81.

In certain embodiments, the instant disclosure provides an antibody that specifically binds to TDP-43 (e.g., human TDP-43), comprising a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to an amino acid sequence set forth in any one of SEQ ID NOs: 82-105. In certain embodiments, the instant disclosure provides an antibody that specifically binds to TDP-43 (e.g., human TDP-43), comprising a VL comprising an amino acid sequence set forth in any one of SEQ ID NOs: 82-105. In certain embodiments, the amino acid sequence of the VL consists of the amino acid sequence set forth in any one of SEQ ID NOs: 82-105.

In certain embodiments, the instant disclosure provides an antibody that specifically binds to TDP-43 (e.g., human TDP-43), comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to an amino acid sequence set forth in any one of SEQ ID NOs: 66-81, and a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to an amino acid sequence set forth in any one of SEQ ID NOs: 82-105. In certain embodiments, the instant disclosure provides an antibody that specifically binds to TDP-43 (e.g., human TDP-43), comprising a VH comprising an amino acid sequence of any one of SEQ ID NOs: 66-81, and a VL comprising an amino acid sequence of any one of SEQ ID NOs: 82-105. In certain embodiments, the amino acid sequence of the VH consists of the amino acid sequence set forth in any one of SEQ ID NOs: 66-81; and the amino acid sequence of the VL consists of the amino acid sequence set forth in any one of SEQ ID NOs: 82-105.

In certain embodiments, the instant disclosure provides an antibody that specifically binds to TDP-43 (e.g., human TDP-43), comprising the VH and VL amino acid sequences set forth in SEQ ID NOs: 67 and 83; 72 and 90; 77 and 96; 66 and 82; 68 and 84; 69 and 85; 68 and 86; 68 and 87; 70 and 88; 71 and 89; 73 and 91; 74 and 92; 75 and 93; 76 and 94; 76 and 95; 76 and 97; 78 and 98; 76 and 99; 79 and 100; 76 and 101; 76 and 102; 76 and 103; 80 and 104; or 81 and 105, respectively. In certain embodiments, the amino acid sequences of VH and VL consist of the amino acid sequences set forth in SEQ ID NOs: SEQ ID NOs: 67 and 83; 72 and 90; 77 and 96; 66 and 82; 68 and 84; 69 and 85; 68 and 86; 68 and 87; 70 and 88; 71 and 89; 73 and 91; 74 and 92; 75 and 93; 76 and 94; 76 and 95; 76 and 97; 78 and 98; 76 and 99; 79 and 100; 76 and 101; 76 and 102; 76 and 103; 80 and 104; or 81 and 105, respectively. In certain embodiments, the VH and VL comprise the amino acid sequences, respectively, set forth in SEQ ID NOs: 67 and 83. In certain embodiments, the VH and VL comprise the amino acid sequences, respectively, set forth in SEQ ID NOs: 72 and 90. In certain embodiments, the VH and VL comprise the amino acid sequences, respectively, set forth in SEQ ID NOs: 77 and 96.

In certain embodiments, the instant disclosure provides an antibody that cross-competes for binding to TDP-43 (e.g., human TDP-43) with an antibody comprising the VH and VL amino acid sequences set forth in SEQ ID NOs: 67 and 83; 72 and 90; 77 and 96; 66 and 82; 68 and 84; 69 and 85; 68 and 86; 68 and 87; 70 and 88; 71 and 89; 73 and 91; 74 and 92; 75 and 93; 76 and 94; 76 and 95; 76 and 97; 78 and 98; 76 and 99; 79 and 100; 76 and 101; 76 and 102; 76 and 103; 80 and 104; or 81 and 105, respectively.

In certain embodiments, the instant disclosure provides an antibody that binds to the same or an overlapping epitope of TDP-43 (e.g., an epitope of human TDP-43) as an antibody described herein, e.g., an antibody comprising the VH and VL amino acid sequences set forth in SEQ ID NOs: 67 and 83; 72 and 90; 77 and 96; 66 and 82; 68 and 84; 69 and 85; 68 and 86; 68 and 87; 70 and 88; 71 and 89; 73 and 91; 74 and 92; 75 and 93; 76 and 94; 76 and 95; 76 and 97; 78 and 98; 76 and 99; 79 and 100; 76 and 101; 76 and 102; 76 and 103; 80 and 104; or 81 and 105, respectively.

In certain embodiments, the epitope of an antibody can be determined by, e.g., NMR spectroscopy, surface plasmon resonance (BIAcore®), X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303, all of which are herein incorporated by reference in their entireties). Antibody: antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H W et al.; U.S. Patent Application No. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323, all of which are herein incorporated by reference in their entireties). Mutagenesis mapping studies may be accomplished using any method known to one of skill in the art. See, e.g., Champe M et al., (1995) supra and Cunningham B C & Wells J A (1989) supra for a description of mutagenesis techniques, including alanine scanning mutagenesis techniques. In a specific embodiment, the epitope of an antibody is determined using alanine scanning mutagenesis studies. In addition, or antibodies that recognize and bind to the same or overlapping epitopes of TDP-43 (e.g., human TDP-43) can be identified using routine techniques such as an immunoassay, for example, by showing the ability of one antibody to block the binding of another antibody to a target antigen, i.e., a competitive binding assay. Competition binding assays also can be used to determine whether two antibodies have similar binding specificity for an epitope. Competitive binding can be determined in an assay in which the immunoglobulin under test inhibits specific binding of a reference antibody to a common antigen, such as TDP-43 (e.g., human TDP-43). Numerous types of competitive binding assays are known, for example: solid phase direct or indirect radioimmunoassay (RIA), solid phase direct or indirect enzyme immunoassay (EIA), sandwich competition assay (see Stahli C et al., (1983) Methods Enzymol 9:242-253); solid phase direct biotin-avidin EIA (see Kirkland T N et al., (1986) J Immunol 137:3614-9); solid phase direct labeled assay, solid phase direct labeled sandwich assay (see Harlow E & Lane D, (1988) Antibodies: A Laboratory Manual, Cold Spring Harbor Press); solid phase direct label RIA using I-125 label (see Morel G A et al., (1988) Mol Immunol 25 (1): 7-15); solid phase direct biotin-avidin EIA (see Cheung R C et al., (1990) Virology 176:546-52); and direct labeled RIA (see Moldenhauer G et al., (1990) Scand J Immunol 32:77-82), all of which are herein incorporated by reference in their entireties. Typically, such an assay involves the use of purified antigen (e.g., TDP-43, such as human TDP-43) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually, the test immunoglobulin is present in excess. Usually, when a competing antibody is present in excess, it will inhibit specific binding of a reference or antibody to a common antigen by at least 50-55%, 55-60%, 60-65%, 65-70%, 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, for example, Wagener C et al., (1983) J Immunol 130:2308-2315; Wagener C et al., (1984) J Immunol Methods 68:269-274; Kuroki M et al., (1990) Cancer Res 50:4872-4879; Kuroki M et al., (1992) Immunol Invest 21:523-538; Kuroki M et al., (1992) Hybridoma 11:391-407 and Antibodies: A Laboratory Manual, Ed Harlow E & Lane D editors supra, pp. 386-389, all of which are herein incorporated by reference in their entireties.

In certain embodiments, the instant disclosure provides an antibody that specifically binds to TDP-43 (e.g., human TDP-43), wherein the antibody binds to a C-terminal region of TDP-43 (SEQ ID NO: 106). In certain embodiments, the instant disclosure provides an antibody that specifically binds to TDP-43 (e.g., human TDP-43), wherein the antibody binds within a region of TDP-43 corresponding to amino acid residues 274-414 of human TDP-43 (SEQ ID NO: 106). In certain embodiments, the instant disclosure provides an antibody that specifically binds to TDP-43 (e.g., human TDP-43), wherein the antibody binds within a region of TDP-43 corresponding to amino acid residues 389-411 of human TDP-43 (SEQ ID NO: 106). In certain embodiments, the instant disclosure provides an antibody that specifically binds to TDP-43 (e.g., human TDP-43), wherein the antibody binds to an epitope of TDP-43 corresponding to amino acid residues 389-411 of human TDP-43 (SEQ ID NO: 106). In certain embodiments, the instant disclosure provides an antibody that specifically binds to TDP-43 (e.g., human TDP-43), wherein the antibody binds within a region of TDP-43 corresponding to amino acid residues 397-411 of human TDP-43 (SEQ ID NO: 106). In certain embodiments, the instant disclosure provides an antibody that specifically binds to TDP-43 (e.g., human TDP-43), wherein the antibody binds to an epitope of TDP-43 corresponding to amino acid residues 397-411 of human TDP-43 (SEQ ID NO: 106).

In certain embodiments, the instant disclosure provides an antibody that specifically binds to TDP-43 (e.g., human TDP-43), wherein the antibody comprises a VH and VL that are covalently linked. In certain embodiments, the C-terminus of the VH is covalently linked to the N-terminus of the VL. In certain embodiments, the C-terminus of the VL is covalently linked to the N-terminus of the VH. In certain embodiments, the VH and VL are covalently linked via a peptide linker.

Exemplary peptide linkers and anti-TDP-43 antibodies wherein the VH and VL are covalently linked (e.g., scFvs) are set forth in Table 2.

TABLE 2
Amino acid sequences of exemplary peptide linkers and anti-TDP-43 antibodies.

| | SEQ ID | |
|---|---|---|
| Construct | NO: | Amino Acid Sequence |
| Linker | 106 | GSGSGSS |
| Linker | 107 | GSGSS |
| Linker | 108 | NS |
| Linker | 109 | AAAYPYDVPDYA |
| Linker | 110 | GSGSG |
| Linker | 111 | GSGSGGSGSG |
| Linker | 112 | GSGSGGSGSGGSGSGGSGSG |
| Linker | 113 | LEGGGGSAAA |
| Linker | 114 | SGS |
| Linker | 115 | GGSGS |
| Linker | 116 | GGGGS |
| Linker | 117 | GGGGSGGGGSGGGGS |
| Linker | 118 | EQKLISEEDLS |
| A | 120 | QVQLVQSGAEVKKPGASVKVSCKASGYAFTGKYIHWVRQAPGQG |
| | | LEWMGGINPNNGGTSYNQKFQGRVTMTRDTSTSTVYMELSSLRSE |
| | | DTAVYYCARKSWGQGTLVTVSSGGGGSGGGGSGGGGSDIVMTQSP |
| | | LSLPVTPGEPASISCRSSKSLLHSNGNTYLYWYLQKPGQSPQLLIYR |
| | | MSNLASGVPDRFSGSGSGTDFTLKISRVEAEDVGVYYCMQGLQTPL |
| | | TFGGGTKVEIK |
| B | 121 | QVQLVQSGAEVKKPGASVKVSCKASGFTFTELSMHWVRQAPGQGL |
| | | EWMGGINPNNGGTSYNQKFQGRVTMTRDTSTSTVYMELSSLRSED |
| | | TAVYYCARESWGQGTLVTVSSGGGGSGGGGSGGGGSDIVMTQSPL |
| | | SLPVTPGEPASISCKSSQSLLHSDGKTYLNWYLQKPGQSPQLLIYLAS |
| | | RRASGVPDRFSGSGSGTDFTLKISRVEAEDVGVYYCFQGTHFPHTFG |
| | | GGTKVEIK |
| C | 122 | QVQLVQSGAEVKKPGASVKVSCKASGFTFTEYSMHWVRQAPGQG |
| | | LEWMGGINPNNGGTSYNQKFQGRVTMTRDTSTSTVYMELSSLRSE |
| | | DTAVYYCARESWGQGTLVTVSSGGGGSGGGGSGGGGSDIVMTQSP |
| | | LSLPVTPGEPASISCKSSQSLLHSDGKTYLNWYLQKPGQSPQLLIYL |
| | | GSNRASGVPDRFSGSGSGTDFTLKISRVEAEDVGVYYCFQGTHFPH |
| | | TFGGGTKVEIK |
| D | 123 | QVQLVQSGAEVKKPGASVKVSCKASGGTFTEYSMHWVRQAPGQG |
| | | LEWMGGINPNNGGTRYNQKFQGRVTMTRDTSTSTVYMELSSLRSE |
| | | DTAVYYCARESWGQGTLVTVSSGGGGSGGGGSGGGGSDIVMTQSP |
| | | LSLPVTPGEPASISCKSSQSLLHSDGKTYLNWYLQKPGQSPQLLIYL |
| | | VSNRASGVPDRFSGSGSGTDFTLKISRVEAEDVGVYYCHQGTHFPH |
| | | TFGGGTKVEIK |
| E | 124 | QVQLVQSGAEVKKPGASVKVSCKASGFTFTEYSMHWVRQAPGQG |
| | | LEWMGGINPNNGGTSYNQKFQGRVTMTRDTSTSTVYMELSSLRSE |
| | | DTAVYYCARESWGQGTLVTVSSGGGGSGGGGSGGGGSDIVMTQSP |
| | | LSLPVTPGEPASISCKSSQSLLHSDGKTYLNWYLQKPGQSPQLLIYL |
| | | GSSRASGVPDRFSGSGSGTDFTLKISRVEAEDVGVYYCMQGTHFPH |
| | | TFGGGTKVEIK |
| F | 125 | QVQLVQSGAEVKKPGASVKVSCKASGFTFTEYSMHWVRQAPGQG |
| | | LEWMGGINPNNGGTSYNQKFQGRVTMTRDTSTSTVYMELSSLRSE |
| | | DTAVYYCARESWGQGTLVTVSSGGGGSGGGGSGGGGSDIVMTQSP |
| | | LSLPVTPGEPASISCKSSQSLLHNDGKTYLNWYLQKPGQSPQLLIYL |
| | | ASNRASGVPDRFSGSGSGTDFTLKISRVEAEDVGVYYCGQGTHFPH |
| | | TFGGGTKVEIK |
| G | 126 | QVQLVQSGAEVKKPGASVKVSCKASGFTFTEYSMHWVRQAPGQG |
| | | LEWMGGINPNNGGTSYNQKFQGRVTMTRDTSTSTVYMELSSLRSE |
| | | DTAVYYCASESWGQGTLVTVSSGGGGSGGGGSGGGGSDIVMTQSP |
| | | LSLPVTPGEPASISCKSSQSLLHSDGKTYLNWYLQKPGQSPQLLIYL |
| | | VSKLKSGVPDRFSGSGSGTDFTLKISRVEAEDVGVYYCMQGTHFPH |
| | | TFGGGTKVEIK |
| H | 127 | QVQLVQSGAEVKKPGASVKVSCKASGFTFTEYSMHWVRQAPGQG |
| | | LEWMGGINPNNGGTRYNQKFQGRVTMTRDTSTSTVYMELSSLRSE |
| | | DTAVYYCARESWGQGTLVTVSSGGGGSGGGGSGGGGSDIVMTQSP |
| | | LSLPVTPGEPASISCKSSQSLLHKDGKTYLNWYLQKPGQSPQLLIYL |
| | | VSKLPSGVPDRFSGSGSGTDFTLKISRVEAEDVGVYYCMQGTHFPH |
| | | TFGGGTKVEIK |
| I | 128 | QVQLVQSGAEVKKPGASVKVSCKASGFTFTEYSMHWVRQAPGQG |
| | | LEWMGGINPNNGGTIYNQKFQGRVTMTRDTSTSTVYMELSSLRSED |
| | | TAVYYCARESWGQGTLVTVSSGGGGSGGGGSGGGGSDIVMTQSPL |
| | | SLPVTPGEPASISCKSSQSLLHSDGKTYLNWYLQKPGQSPQLLIYLGS |
| | | NRASGVPDRFSGSGSGTDFTLKISRVEAEDVGVYYCMQGTHFPHTF |
| | | GGGTKVEIK |
| J | 129 | QVQLVQSGAEVKKPGASVKVSCKASGGTFSSYAISWVRQAPGQGL |
| | | EWMGEINPSNGRTVYNQKFQGRVTMTRDTSTSTVYMELSSLRSED |
| | | TAVYYCARYKDYWGQGTLVTVSSGGGGSGGGGSGGGGSDIVMTQ |
| | | SPLSLPVTPGEPASISCRSSQSIVTAIGNTYLEWYLQKPGQSPQLLIYK |
| | | VSNRFSGVPDRFSGSGSGTDFTLKISRVEAEDVGVYYCFQGSHVPFT |
| | | FGGGTKVEIK |
| K | 130 | QVQLVQSGAEVKKPGASVKVSCKASGGTFSSYAIHWVRQAPGQGL |
| | | EWMGEINPSNGRTNYNQKFQGRVTMTRDTSTSTVYMELSSLRSED |
| | | TAVYYCARRMDYWGQGTLVTVSSGGGGSGGGGSGGGGSDIVMTQ |
| | | SPLSLPVTPGEPASISCRSSKSLLHSNGNTYLYWYLQKPGQSPQLLIY |
| | | RMSNRASGVPDRFSGSGSGTDFTLKISRVEAEDVGVYYCMQALQTP |
| | | FTFGGGTKVEIK |
| L | 131 | QVQLVQSGAEVKKPGASVKVSCKASGYTFTKYWMHWVRQAPGQ |
| | | GLEWVGSMNPNSGHTGFAQKFQGRVTMTRDTSTSTVYMELSSLRS |
| | | EDTAVYYCARAMDYWGQGTLVTVSSGGGGSGGGGSGGGGSDIVM |
| | | TQSPLSLPVTPGEPASISCKSSQSLLHSDGKTYLYWYLQKPGQSPQL |
| | | LIYKVSNRFKGVPDRFSGSGSGTDFTLKISRVEAEDVGVYYCMQKI |
| | | QLPVTFGGGTKVEIK |
| M | 132 | QVQLVQSGAEVKKPGASVKVSCKASGYTFTKYWMHWVRQAPGQ |
| | | GLEWMGEINPSNGRTNYNQKFQGRVTMTRDTSTSTVYMELSSLRS |
| | | EDTAVYYCWRYMDYWGQGTLVTVSSGGGGSGGGGSGGGGSDIV |
| | | MTQSPLSLPVTPGEPASISCRSSESIVHSNGNTYLEWYLQKPGQSPQL |
| | | LIYKVSNKFSGVPDRFSGSGSGTDFTLKISRVEAEDVGVYYCMQYT |
| | | HWPPTFGGGTKVEIK |
| N | 133 | QVQLVQSGAEVKKPGASVKVSCKASGYTFTKYWMHWVRQAPGQ |
| | | GLEWMGEINPSNGRTNYNQKFQGRVTMTRDTSTSTVYMELSSLRS |
| | | EDTAVYYCWRYMDYWGQGTLVTVSSGGGGSGGGGSGGGGSDIV |
| | | MTQSPLSLPVTPGEPASISCRSSQSLLHSNGYNYLDWYLQKPGQSPQ |
| | | LLIYKVENRFSGVPDRFSGSGSGTDFTLKISRVEAEDVGVYYCMQH |
| | | THWPPTFGGGTKVEIK |
| O | 134 | QVQLVQSGAEVKKPGASVKVSCKASGYTFTKYWMHWVRQAPGQ |
| | | GLEWMGEINPKNGRTNYNQKFQGRVTMTRDTSTSTVYMELSSLRS |
| | | EDTAVYYCWRYMDYWGQGTLVTVSSGGGGSGGGGSGGGGSDIV |
| | | MTQSPLSLPVTPGEPASISCRSSQSLLHSNGYNYLDWYLQKPGQSPQ |
| | | LLIYKVSNRFSGVPDRFSGSGSGTDFTLKISRVEAEDVGVYYCLQHT |
| | | HWPPTFGGGTKVEIK |
| P | 135 | QVQLVQSGAEVKKPGASVKVSCKASGYTFTKYWMHWVRQAPGQ |
| | | GLEWMGEINPSNGRTNYNQKFQGRVTMTRDTSTSTVYMELSSLRS |
| | | EDTAVYYCWRYMDYWGQGTLVTVSSGGGGSGGGGSGGGGSDIV |
| | | MTQSPLSLPVTPGEPASISCRSSQSLLHSNGYNYLDWYLQKPGQSPQ |
| | | LLIYEVSNRFSGVPDRFSGSGSGTDFTLKISRVEAEDVGVYYCMQHT |
| | | HWPPTFGGGTKVEIK |
| Q | 136 | QVQLVQSGAEVKKPGASVKVSCKASGYTFTKYWMHWVRQAPGQ |
| | | GLEWMGEINPQNGRTNYNQKFQGRVTMTRDTSTSTVYMELSSLRS |
| | | EDTAVYYCWRYMDYWGQGTLVTVSSGGGGSGGGGSGGGGSDIV |
| | | MTQSPLSLPVTPGEPASISCRSSRSLLHSNGYNYLDWYLQKPGQSPQ |
| | | LLIYAASSLQSGVPDRFSGSGSGTDFTLKISRVEAEDVGVYYCLQHT |
| | | HWPPTFGGGTKVEIK |
| R | 137 | QVQLVQSGAEVKKPGASVKVSCKASGYTFTKYWMHWVRQAPGQ |
| | | GLEWMGEINPSNGRTNYNQKFQGRVTMTRDTSTSTVYMELSSLRS |
| | | EDTAVYYCWRYMDYWGQGTLVTVSSGGGGSGGGGSGGGGSDIV |
| | | MTQSPLSLPVTPGEPASISCRSSQSLLHSNGYNYLDWYLQKPGQSPQ |
| | | LLIYKVSNRFSGVPDRFSGSGSGTDFTLKISRVEAEDVGVYYCMQH |
| | | THWPPTFGGGTKVEIK |
| S | 138 | QVQLVQSGAEVKKPGASVKVSCKASGYTFTKYWMHWVRQAPGQ |
| | | GLEWMGEINPSNGRTNYPQKFQGRVTMTRDTSTSTVYMELSSLRSE |
| | | DTAVYYCWRYMDYWGQGTLVTVSSGGGGSGGGGSGGGGSDIVM |
| | | TQSPLSLPVTPGEPASISCRSSQSLLHSNGYNYLDWYLQKPGQSPQL |
| | | LIYKVSNRFTGVPDRFSGSGSGTDFTLKISRVEAEDVGVYYCMQHT |
| | | HWPPAFGGGTKVEIK |
| T | 139 | QVQLVQSGAEVKKPGASVKVSCKASGYTFTKYWMHWVRQAPGQ |
| | | GLEWMGEINPSNGRTNYNQKFQGRVTMTRDTSTSTVYMELSSLRS |
| | | EDTAVYYCWRYMDYWGQGTLVTVSSGGGGSGGGGSGGGGSDIV |
| | | MTQSPLSLPVTPGEPASISCRSSQSLLHSNGYNYLDWYLQKPGQSPQ |
| | | LLIYKSSNRFSGVPDRFSGSGSGTDFTLKISRVEAEDVGVYYCMQHT |
| | | HWPPTFGGGTKVEIK |
| U | 140 | QVQLVQSGAEVKKPGASVKVSCKASGYTFTKYWMHWVRQAPGQ |
| | | GLEWMGEINPSNGRTNYNQKFQGRVTMTRDTSTSTVYMELSSLRS |
| | | EDTAVYYCWRYMDYWGQGTLVTVSSGGGGSGGGGSGGGGSDIV |
| | | MTQSPLSLPVTPGEPASISCRSSQSLLNSNGVTFVEWYLQKPGQSPQ |
| | | LLIYEVSNRFSGVPDRFSGSGSGTDFTLKISRVEAEDVGVYYCLQHT |
| | | HWPPTFGGGTKVEIK |
| V | 141 | QVQLVQSGAEVKKPGASVKVSCKASGYTFTKYWMHWVRQAPGQ |
| | | GLEWMGEINPSNGRTNYNQKFQGRVTMTRDTSTSTVYMELSSLRS |
| | | EDTAVYYCWRYMDYWGQGTLVTVSSGGGGSGGGGSGGGGSDIV |
| | | MTQSPLSLPVTPGEPASISCRSSQSLLHSNGYNYLDWYLQKPGQSPQ |
| | | LLIYEVSNRFWGVPDRFSGSGSGTDFTLKISRVEAEDVGVYYCLQH |
| | | THWPPTFGGGTKVEIK |
| W | 142 | QVQLVQSGAEVKKPGASVKVSCKASGYTFTKYWMHWVRQAPGQ |
| | | GLEWMGGINPSNGRTNYNQKFQGRVTMTRDTSTSTVYMELSSLRS |
| | | EDTAVYYCARYMQYWGQGTLVTVSSGGGGSGGGGSGGGGSDIVM |
| | | TQSPLSLPVTPGEPASISCRSSKSLLHSNGNTYLYWYLQKPGQSPQL |
| | | LIYKVSNQSSGVPDRFSGSGSGTDFTLKISRVEAEDVGVYYCMQAL |
| | | QTPLTFGGGTKVEIK |
| X | 143 | QVQLVQSGAEVKKPGASVKVSCKASGYTFTSHYLHWVRQAPGQG |
| | | LEWMGIINPSNGRTNYNQKFQGRVTMTRDTSTSTVYMELSSLRSED |
| | | TAVYYCWRYMDYWGQGTLVTVSSGGGGSGGGGSGGGGSDIVMT |
| | | QSPLSLPVTPGEPASISCRSSQSLLHSNGYNYLDWYLQKPGQSPQLLI |
| | | YEVSNRFSGVPDRFSGSGSGTDFTLKISRVEAEDVGVYYCLQHTHW |
| | | PPTFGGGTKVEIK |

In certain embodiments, the peptide linker is 1-25 amino acids in length. In certain embodiments, the peptide linker comprises an amino acid sequence selected from the group consisting of SEQ ID NOs: 106-118. In certain embodiments, the peptide linker consists of an amino acid sequence selected from the group consisting of SEQ ID NOs: 106-118.

In certain embodiments, the instant disclosure provides an antibody that specifically binds to TDP-43 (e.g., human TDP-43), wherein the antibody is an scFv. In certain embodiments, the antibody comprises an amino acid sequence at least 75%, 80%, 85%, 90%, 95%, or 100% (e.g., at least 86, 87, 88, 89, 90, 91, 92, 93, 94, 95, 96, 97, 98, or 99%) identical to an amino acid sequence selected from the group consisting of SEQ ID NOs: 120-143. In certain embodiments, the antibody comprises an amino acid sequence selected from the group consisting of SEQ ID NOs: 120-143. In certain embodiments, the antibody consists of an amino acid sequence selected from the group consisting of SEQ ID NOs: 120-143.

In certain embodiments, the instant disclosure provides a polypeptide comprising a VH of any antibody described herein. In certain embodiments, the instant disclosure provides a polypeptide comprising a VH comprising the CDRH1, CDRH2, and CDRH3 amino acid sequences of any one of the VH amino acid sequences set forth in SEQ ID NOs: 66-81. In certain embodiments, the polypeptide comprises a VH comprising an amino acid sequence set forth in any one of SEQ ID NOs: 66-81. In certain embodiments, the polypeptide comprises a VH consisting of an amino acid sequence set forth in any one of SEQ ID NOs: 66-81.

In certain embodiments, the instant disclosure provides a polypeptide comprising a VL of any antibody described herein. In certain embodiments, the instant disclosure provides a polypeptide comprising a VL comprising the CDRH1, CDRH2, and CDRH3 amino acid sequences of any one of the VL amino acid sequences set forth in SEQ ID NOs: 82-105. In certain embodiments, the polypeptide comprises a VL comprising an amino acid sequence set forth in any one of SEQ ID NOs: 82-105. In certain embodiments, the polypeptide comprises a VL consisting of an amino acid sequence set forth in any one of SEQ ID NOs: 82-105.

In certain embodiments, the instant disclosure provides a polypeptide comprising a VH and a VL of any antibody described herein. In certain embodiments, the instant disclosure provides a polypeptide comprising a VH comprising the CDRH1, CDRH2, and CDRH3 amino acid sequences of any one of the VH amino acid sequences set forth in SEQ ID NOs: 66-81, and a VL comprising the CDRH1, CDRH2, and CDRH3 amino acid sequences of any one of the VL amino acid sequences set forth in SEQ ID NOs: 82-105. In certain embodiments, the polypeptide comprises a VH comprising an amino acid sequence set forth in any one of SEQ ID NOs: 66-81, and a VL comprising an amino acid sequence set forth in any one of SEQ ID NOs: 82-105. In certain embodiments, the polypeptide comprises a VH consisting of an amino acid sequence set forth in any one of SEQ ID NOs: 66-81, and a VL consisting of an amino acid sequence set forth in any one of SEQ ID NOs: 82-105.

In certain embodiments, the instant disclosure provides a polypeptide comprising an amino acid sequence selected from the group consisting of SEQ ID NOs: 66-105 and 120-143. In certain embodiments, the instant disclosure provides a polypeptide consisting of an amino acid sequence selected from the group consisting of SEQ ID NOs: 66-105 and 120-143.

The anti-TDP-43 (e.g., human TDP-43) antigen-binding molecules of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bispecific or a multispecific antibody with a second or additional binding specificity.

In certain embodiments, the antibody disclosed herein is conjugated to a cytotoxic agent, cytostatic agent, toxin, radionuclide, or detectable label. In certain embodiments, the cytotoxic agent is able to induce death or destruction of a cell in contact therewith. In certain embodiments, the cytostatic agent is able to prevent or substantially reduce proliferation and/or inhibits the activity or function of a cell in contact therewith. In certain embodiments, the cytotoxic agent or cytostatic agent is a chemotherapeutic agent. In certain embodiments, the radionuclide is selected from the group consisting of the isotopes ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac, and ¹⁸⁶Re. In certain embodiments, the detectable label comprises a fluorescent moiety or a click chemistry handle.

Any immunoglobulin (Ig) constant region can be used in the antibodies disclosed herein. In certain embodiments, the Ig region is a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

In certain embodiments, the instant disclosure provides an antibody that specifically binds to TDP-43 (e.g., human TDP-43), the antibody comprising a heavy chain constant region, optionally selected from the group consisting of human IgG1, IgG2, IgG3, IgG4, IgA1, IgA2, and IgM.

In certain embodiments, the instant disclosure provides an antibody that specifically binds to TDP-43 (e.g., human TDP-43), the antibody comprising a heavy chain constant region that is a variant of a wild-type heavy chain constant region, wherein the variant heavy chain constant region binds to an FcγR with lower affinity than the wild-type heavy chain constant region binds to the FcγR.

In certain embodiments, one, two, or more mutations (e.g., amino acid substitutions) are introduced into an Fc region (e.g., a CH2 domain (residues 231-340 of human IgG1)) and/or a CH3 domain (residues 341-447 of human IgG1, numbered according to the EU numbering system) and/or a hinge region (residues 216-230, numbered according to the EU numbering system) of an antibody described herein, to alter one or more functional properties of the antibody, such as serum half-life, complement fixation, Fc receptor binding, and/or antigen-dependent cellular cytotoxicity.

In certain embodiments, one, two, or more mutations (e.g., amino acid substitutions) are introduced into the hinge region of an antibody described herein, such that the number of cysteine residues in the hinge region is altered (e.g., increased or decreased) as described in, e.g., U.S. Pat. No. 5,677,425, herein incorporated by reference in its entirety. The number of cysteine residues in the hinge region may be altered to, e.g., facilitate assembly of the light and heavy chains, or to alter (e.g., increase or decrease) the stability of the antibody.

In a specific embodiment, one, two, or more amino acid mutations (e.g., substitutions, insertions, or deletions) are introduced into an IgG constant region, or FcRn-binding fragment thereof (preferably an Fc or hinge-Fc fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745, all of which are herein incorporated by reference in their entireties, for examples of mutations that will alter (e.g., decrease or increase) the half-life of an antibody in vivo. In certain embodiments, one, two or more amino acid mutations (e.g., substitutions, insertions, or deletions) are introduced into an IgG constant region, or FcRn-binding fragment thereof (preferably an Fc or hinge-Fc fragment) to decrease the half-life of the antibody in vivo. In other embodiments, one, two or more amino acid mutations (e.g., substitutions, insertions, or deletions) are introduced into an IgG constant region, or FcRn-binding fragment thereof (preferably an Fc or hinge-Fc fragment) to increase the half-life of the antibody in vivo. In a specific embodiment, the antibodies may have one or more amino acid mutations (e.g., substitutions) in the second constant (CH2) domain (residues 231-340 of human IgG1) and/or the third constant (CH3) domain (residues 341-447 of human IgG1), numbered according to the EU numbering system. In a specific embodiment, the constant region of the IgG1 of antibody described herein comprises a methionine (M) to tyrosine (Y) substitution in position 252, a serine(S) to threonine (T) substitution in position 254, and a threonine (T) to glutamic acid (E) substitution in position 256, numbered according to the EU numbering system. See U.S. Pat. No. 7,658,921, which is herein incorporated by reference in its entirety. This type of mutant IgG, referred to as “YTE mutant” has been shown to display fourfold increased half-life as compared to wild-type versions of the same antibody (see Dall'Acqua W F et al., (2006) J Biol Chem 281:23514-24, which is herein incorporated by reference in its entirety). In certain embodiments, an antibody comprises an IgG constant region comprising one, two, three or more amino acid substitutions of amino acid residues at positions 251-257, 285-290, 308-314, 385-389, and 428-436, numbered according to the EU numbering system.

In certain embodiments, one, two, or more mutations (e.g., amino acid substitutions) are introduced into an Fc region (e.g., a CH2 domain (residues 231-340 of human IgG1)) and/or a CH3 domain (residues 341-447 of human IgG1, numbered according to the EU numbering system) and/or a hinge region (residues 216-230, numbered according to the EU numbering system) of an antibody described herein, to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, all of which are herein incorporated by reference in their entireties.

In certain embodiments, the antibody comprises a heavy chain constant region that is a variant of a wild-type heavy chain constant region, wherein the variant heavy chain constant region binds to FcγRIIB with higher affinity than the wild-type heavy chain constant region binds to FcγRIIB. In certain embodiments, the variant heavy chain constant region is a variant human heavy chain constant region, e.g., a variant human IgG1, a variant human IgG2, or a variant human IgG4 heavy chain constant region. In certain embodiments, the variant human IgG heavy chain constant region comprises one or more of the following amino acid mutations, according to the EU numbering system: G236D, P238D, S239D, S267E, L328F, and L328E. In certain embodiments, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F, according to the EU numbering system. In certain embodiments, the FcγRIIB is expressed on a cell selected from the group consisting of macrophages, monocytes, B cells, dendritic cells, endothelial cells, and activated T cells.

In a further embodiment, one, two, or more amino acid substitutions are introduced into an IgG constant region Fc region to alter the effector function(s) of the antibody. For example, one or more amino acids selected from amino acid residues 234, 235, 236, 237, 239, 243, 267, 292, 297, 300, 318, 320, 322, 328, 330, 332, and 396, numbered according to the EU numbering system, can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In certain embodiments, the deletion or inactivation (through point mutations or other means) of a constant region domain may reduce Fc receptor binding of the circulating antibody thereby increasing tumor localization. See, e.g., U.S. Pat. Nos. 5,585,097 and 8,591,886, each of which is herein incorporated by reference in its entirety, for a description of mutations that delete or inactivate the constant region and thereby increase tumor localization. In certain embodiments, one or more amino acid substitutions may be introduced into the Fc region of an antibody described herein to remove potential glycosylation sites on the Fc region, which may reduce Fc receptor binding (see, e.g., Shields R L et al., (2001) J Biol Chem 276:6591-604, which is herein incorporated by reference in its entirety). In various embodiments, one or more of the following mutations in the constant region of an antibody described herein may be made: an N297A substitution; an N297Q substitution; an L234A substitution; an L234F substitution; an L235A substitution; an L235F substitution; an L235V substitution; an L237A substitution; an S239D substitution; an E233P substitution; an L234V substitution; a C236 deletion; a P238A substitution; an F243L substitution; a D265A substitution; an S267E substitution; an L328F substitution; an R292P substitution; a Y300L substitution; an A327Q substitution; a P329A substitution; an A330L substitution; an 1332E substitution; or a P396L substitution, numbered according to the EU numbering system.

In certain embodiments, a mutation selected from the group consisting of D265A, P329A, and a combination thereof, numbered according to the EU numbering system, may be made in the constant region of an antibody described herein. In certain embodiments, a mutation selected from the group consisting of L235A, L237A, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein. In certain embodiments, a mutation selected from the group consisting of S239D, 1332E, optionally A330L, and a combination thereof, numbered according to the EU numbering system, may be made in the constant region of an antibody described herein. In certain embodiments, a mutation selected from the group consisting of L235V, F243L, R292P, Y300L, P396L, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein.

In a specific embodiment, an antibody described herein comprises the constant region of an IgG1 with an N297Q or N297A amino acid substitution, numbered according to the EU numbering system. In certain embodiments, an antibody described herein comprises the constant region of an IgG1 with a mutation selected from the group consisting of D265A, P329A, and a combination thereof, numbered according to the EU numbering system. In another embodiment, an antibody described herein comprises the constant region of an IgG1 with a mutation selected from the group consisting of L234A, L235A, and a combination thereof, numbered according to the EU numbering system. In another embodiment, an antibody described herein comprises the constant region of an IgG1 with a mutation selected from the group consisting of L234F, L235F, N297A, and a combination thereof, numbered according to the EU numbering system. In certain embodiments, amino acid residues in the constant region of an antibody described herein in the positions corresponding to positions L234, L235, and D265 in a human IgG1 heavy chain, numbered according to the EU numbering system, are not L, L, and D, respectively. This approach is described in detail in International Publication No. WO 14/108483, which is herein incorporated by reference in its entirety. In certain embodiments, the amino acids corresponding to positions L234, L235, and D265 in a human IgG1 heavy chain are F, E, and A; or A, A, and A, respectively, numbered according to the EU numbering system.

In certain embodiments, one or more amino acids selected from amino acid residues 329, 331, and 322 in the constant region of an antibody described herein, numbered according to the EU numbering system, can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al.), which is herein incorporated by reference in its entirety. In certain embodiments, one or more amino acid residues within amino acid positions 231 to 238 in the N-terminal region of the CH2 domain of an antibody described herein are altered to thereby alter the ability of the antibody to fix complement, numbered according to the EU numbering system. This approach is described further in International Publication No. WO 94/29351, which is herein incorporated by reference in its entirety. In certain embodiments, the Fc region of an antibody described herein is modified to increase the ability of the antibody to mediate antibody dependent cellular cytotoxicity (ADCC) and/or to increase the affinity of the antibody for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU numbering system. This approach is described further in International Publication No. WO 00/42072, which is herein incorporated by reference in its entirety.

In certain embodiments, an antibody described herein comprises a modified constant region of an IgG1, wherein the modification increases the ability of the antibody to mediate antibody dependent cellular cytotoxicity (ADCC). In certain embodiments, 0.1, 1, or 10 μg/mL of the antibody is capable of inducing cell death of at least 20%, 25%, 30%, 35%, 40%, 45%, 50%, 55%, or 60% of TDP-43-expressing cells within 1, 2, or 3 hours, as assessed by methods described herein and/or known to a person of skill in the art. In certain embodiments, the modified constant region of an IgG1 comprises S239D and I332E substitutions, numbered according to the EU numbering system. In certain embodiments, the modified constant region of an IgG1 comprises S239D, A330L, and 1332E substitutions, numbered according to the EU numbering system. In certain embodiments, the modified constant region of an IgG1 comprises L235V, F243L, R292P, Y300L, and P396L substitutions, numbered according to the EU numbering system. In certain embodiments, the antibody is capable of inducing cell death in effector T cells and Tregs, wherein the percentage of Tregs that undergo cell death is higher than the percentage of effector T cells that undergo cell death by at least 1.2 fold, 1.3 fold, 1.4 fold, 1.5 fold, 1.6 fold, 1.7 fold, 1.8 fold, 1.9 fold, 2 fold, 2.5 fold, 3 fold, 3.5 fold, 4 fold, 4.5 fold, or 5 fold.

In certain embodiments, any of the constant region mutations or modifications described herein can be introduced into one or both heavy chain constant regions of an antibody described herein having two heavy chain constant regions.

### Polynucleotides, Vectors, and Methods of Producing Antibodies

In one aspect, provided herein are: polynucleotides comprising a nucleotide sequence encoding an antibody described herein that specifically binds to a TDP-43 (e.g., human TDP-43); vectors (e.g., expression vectors (e.g., AAV vectors)) comprising such polynucleotides; and method of producing such antibodies. Polynucleotide sequences of exemplary anti-TDP-43 antibodies are set forth in Table 3.

TABLE 3
Polynucleotide sequences of exemplary anti-TDP-43 antibodies.

| | SEQ ID | |
|---|---|---|
| Construct | NO: | VH Sequence |
| A | 144 | CAGGTGCAGCTGGTGCAGTCTGGGGCTGAGGTGAAGAAGCCTGG |
| | | GGCCTCAGTGAAGGTTTCCTGCAAGGCATCTGGATACGCATTCA |
| | | CCGGGAAGTATATCCACTGGGTGCGACAGGCCCCTGGACAAGGG |
| | | CTTGAGTGGATGGGAGGAATCAACCCAAATAACGGTGGCACAA |
| | | GCTACAATCAGAAGTTCCAGGGCAGAGTCACCATGACCAGGGAC |
| | | ACGTCCACGAGCACAGTCTACATGGAGCTGAGCAGCCTGAGATC |
| | | TGAGGACACGGCCGTGTATTACTGTGCTAGAAAATCCTGGGGCC |
| | | AAGGAACCCTGGTCACCGTCTCCTCA |
| B | 145 | CAGGTGCAGCTGGTGCAGTCTGGGGCTGAGGTGAAGAAGCCTGG |
| | | GGCCTCAGTGAAGGTTTCCTGCAAGGCATCTGGATTTACCTTCAC |
| | | CGAGCTGTCCATGCACTGGGTGCGACAGGCCCCTGGACAAGGGC |
| | | TTGAGTGGATGGGAGGAATCAACCCTAATAACGGTGGCACAAGC |
| | | TACAATCAGAAGTTCCAGGGCAGAGTCACCATGACCAGGGACAC |
| | | GTCCACGAGCACAGTCTACATGGAGCTGAGCAGCCTGAGATCTG |
| | | AGGACACGGCCGTGTATTACTGTGCTAGAGAATCCTGGGGCCAA |
| | | GGAACCCTGGTCACCGTCTCCTCA |
| C | 146 | CAGGTGCAGCTGGTGCAGTCTGGGGCTGAGGTGAAGAAGCCTGG |
| | | GGCCTCAGTGAAGGTTTCCTGCAAGGCATCTGGATTCACCTTCAC |
| | | CGAGTACAGTATGCACTGGGTGCGACAGGCCCCTGGACAAGGGC |
| | | TTGAGTGGATGGGAGGCATCAACCCTAACAACGGTGGCACAAGC |
| | | TACAACCAGAAGTTCCAGGGCAGAGTCACCATGACCAGGGACAC |
| | | GTCCACGAGCACAGTCTACATGGAGCTGAGCAGCCTGAGATCTG |
| | | AGGACACGGCCGTGTATTACTGTGCTAGAGAGTCCTGGGGCCAA |
| | | GGAACCCTGGTCACCGTCTCCTCA |
| D | 147 | CAGGTGCAGCTGGTGCAGTCTGGGGCTGAGGTGAAGAAGCCTGG |
| | | GGCCTCAGTGAAGGTTTCCTGCAAGGCATCTGGAGGCACCTTCA |
| | | CCGAGTACTCTATGCACTGGGTGCGACAGGCCCCTGGACAAGGG |
| | | CTTGAGTGGATGGGAGGAATCAACCCCAATAACGGTGGCACACG |
| | | CTACAACCAGAAGTTCCAGGGCAGAGTCACCATGACCAGGGACA |
| | | CGTCCACGAGCACAGTCTACATGGAGCTGAGCAGCCTGAGATCT |
| | | GAGGACACGGCCGTGTATTACTGTGCTAGAGAATCCTGGGGCCA |
| | | AGGAACCCTGGTCACCGTCTCCTCA |
| E | 148 | CAGGTGCAGCTGGTGCAGTCTGGGGCTGAGGTGAAGAAGCCTGG |
| | | GGCCTCAGTGAAGGTTTCCTGCAAGGCATCTGGATTCACCTTCAC |
| | | CGAGTACTCCATGCACTGGGTGCGACAGGCCCCTGGACAAGGGC |
| | | TTGAGTGGATGGGAGGAATCAACCCAAACAACGGTGGCACAAG |
| | | CTACAACCAGAAGTTCCAGGGCAGAGTCACCATGACCAGGGACA |
| | | CGTCCACGAGCACAGTCTACATGGAGCTGAGCAGCCTGAGATCT |
| | | GAGGACACGGCCGTGTATTACTGTGCTAGAGAATCCTGGGGCCA |
| | | AGGAACCCTGGTCACCGTCTCCTCA |
| F | 149 | CAGGTGCAGCTGGTGCAGTCTGGGGCTGAGGTGAAGAAGCCTGG |
| | | GGCCTCAGTGAAGGTTTCCTGCAAGGCATCTGGATTCACCTTCAC |
| | | CGAATACAGCATGCACTGGGTGCGACAGGCCCCTGGACAAGGGC |
| | | TTGAGTGGATGGGAGGAATCAACCCCAATAATGGTGGAACAAGC |
| | | TACAATCAGAAGTTCCAGGGCAGAGTCACCATGACCAGGGACAC |
| | | GTCCACGAGCACAGTCTACATGGAGCTGAGCAGCCTGAGATCTG |
| | | AGGACACGGCCGTGTATTACTGTGCTAGAGAATCCTGGGGCCAA |
| | | GGAACCCTGGTCACCGTCTCCTCA |
| G | 150 | CAGGTGCAGCTGGTGCAGTCTGGGGCTGAGGTGAAGAAGCCTGG |
| | | GGCCTCAGTGAAGGTTTCCTGCAAGGCATCTGGATTCACCTTCAC |
| | | CGAGTACTCAATGCACTGGGTGCGACAGGCCCCTGGACAAGGGC |
| | | TTGAGTGGATGGGAGGGATCAACCCAAACAACGGTGGAACAAG |
| | | CTACAATCAGAAGTTCCAGGGCAGAGTCACCATGACCAGGGACA |
| | | CGTCCACGAGCACAGTCTACATGGAGCTGAGCAGCCTGAGATCT |
| | | GAGGACACGGCCGTGTATTACTGTGCTTCCGAGTCCTGGGGCCA |
| | | AGGAACCCTGGTCACCGTCTCCTCA |
| H | 151 | CAGGTGCAGCTGGTGCAGTCTGGGGCTGAGGTGAAGAAGCCTGG |
| | | GGCCTCAGTGAAGGTTTCCTGCAAGGCATCTGGATTCACCTTCAC |
| | | CGAGTACTCCATGCACTGGGTGCGACAGGCCCCTGGACAAGGGC |
| | | TTGAGTGGATGGGAGGAATCAACCCAAACAATGGTGGAACAAG |
| | | GTACAACCAGAAGTTCCAGGGCAGAGTCACCATGACCAGGGAC |
| | | ACGTCCACGAGCACAGTCTACATGGAGCTGAGCAGCCTGAGATC |
| | | TGAGGACACGGCCGTGTATTACTGTGCTAGAGAATCCTGGGGCC |
| | | AAGGAACCCTGGTCACCGTCTCCTCA |
| I | 152 | CAGGTGCAGCTGGTGCAGTCTGGGGCTGAGGTGAAGAAGCCTGG |
| | | GGCCTCAGTGAAGGTTTCCTGCAAGGCATCTGGATTTACCTTCAC |
| | | CGAGTACTCGATGCACTGGGTGCGACAGGCCCCTGGACAAGGGC |
| | | TTGAGTGGATGGGAGGCATCAACCCAAACAACGGTGGCACAATC |
| | | TACAACCAGAAGTTCCAGGGCAGAGTCACCATGACCAGGGACAC |
| | | GTCCACGAGCACAGTCTACATGGAGCTGAGCAGCCTGAGATCTG |
| | | AGGACACGGCCGTGTATTACTGTGCTAGAGAGTCCTGGGGCCAA |
| | | GGAACCCTGGTCACCGTCTCCTCA |
| J | 153 | CAAGTGCAGCTGGTGCAGAGCGGCGCCGAGGTGAAGAAGCCCG |
| | | GCGCAAGCGTGAAGGTGAGCTGCAAGGCAAGCGGCGGCACCTT |
| | | CAGCAGCTACGCCATCAGCTGGGTGAGACAAGCCCCCGGCCAAG |
| | | GCCTGGAGTGGATGGGCGAGATCAACCCTAGCAACGGCAGAAC |
| | | CGTGTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCGCTAGATACAAGGATT |
| | | ATTGGGGGCAAGGCACCTTGGTTACCGTTAGCAGC |
| K | 154 | CAAGTGCAGCTGGTGCAGAGCGGCGCCGAGGTGAAGAAGCCCG |
| | | GCGCAAGCGTGAAGGTGAGCTGCAAGGCAAGCGGCGGCACCTT |
| | | CAGCAGCTACGCCATCCACTGGGTGAGACAAGCCCCCGGCCAAG |
| | | GCCTGGAGTGGATGGGCGAGATCAACCCTAGCAACGGCAGAAC |
| | | CAACTACAATCAGAAGTTCCAAGGCAGAGTGACCATGACAAGA |
| | | GACACAAGCACAAGCACCGTGTACATGGAACTGAGCAGCCTGA |
| | | GAAGCGAGGACACCGCCGTATATTACTGCGCTAGAAGAATGGAC |
| | | TATTGGGGCCAAGGCACCCTGGTAACAGTATCCTCC |
| L | 155 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGGTGGGCAGCATGAACCCCAACAGCGGCCACACCG |
| | | GCTTCGCTCAGAAGTTCCAAGGCAGAGTGACCATGACAAGAGAC |
| | | ACAAGCACAAGCACCGTGTACATGGAACTGAGCAGCCTGAGAA |
| | | GCGAGGACACCGCCGTATATTACTGCGCTAGAGCTATGGACTAT |
| | | TGGGGGCAAGGCACCCTGGTGACCGTGTCATCC |
| M | 156 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTATATTACTGCTGGAGATACATGGACT |
| | | ATTGGGGCCAAGGCACCCTGGTAACAGTATCCTCC |
| N | 157 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGC |
| O | 158 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCCAAGAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGC |
| P | 159 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGC |
| Q | 160 | CAAGTGCAGCTGGTCCAAAGCGGTGCAGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTCAGAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTATATTACTGCTGGAGATACATGGACT |
| | | ATTGGGGGCAAGGCACCCTGGTGACCGTGTCAAGC |
| R | 161 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGC |
| S | 162 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACCCTCAGAAGTTCCAAGGCAGAGTGACCATGACAAGAGA |
| | | CACAAGCACAAGCACCGTGTACATGGAACTGAGCAGCCTGAGA |
| | | AGCGAGGACACCGCCGTATATTACTGCTGGAGATACATGGACTA |
| | | TTGGGGCCAAGGCACCCTGGTAACAGTATCCTCC |
| T | 163 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGC |
| U | 164 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGC |
| V | 165 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTATATTACTGCTGGAGATACATGGACT |
| | | ATTGGGGCCAAGGCACCCTGGTAACAGTATCCTCC |
| W | 166 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGGCATCAACCCTAGCAACGGCAGAACCA |
| | | ACTACAATCAGAAGTTCCAAGGCAGAGTGACCATGACAAGAGA |
| | | CACAAGCACAAGCACCGTGTACATGGAACTGAGCAGCCTGAGA |
| | | AGCGAGGACACCGCCGTATATTACTGCGCTAGATACATGCAGTA |
| | | TTGGGGCCAAGGCACCCTGGTAACAGTATCCTCC |
| | 167 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACAAGCCACTACCTGCACTGGGTGAGACAAGCCCCCGGCCAAGG |
| | | CCTGGAGTGGATGGGCATCATCAACCCTAGCAACGGCAGAACCA |
| | | ACTACAATCAGAAGTTCCAAGGCAGAGTGACCATGACAAGAGA |
| | | CACAAGCACAAGCACCGTGTACATGGAACTGAGCAGCCTGAGA |
| | | AGCGAGGACACCGCCGTTTACTACTGCTGGAGATACATGGATTA |
| | | TTGGGGGCAAGGCACCTTGGTTACCGTTAGCAGC |
| | SEQ ID | |
| Construct | NO: | VL Sequence |
| A | 168 | GATATTGTGATGACTCAGTCTCCACTCTCCCTGCCCGTCACCCCT |
| | | GGAGAGCCGGCCTCCATCTCCTGCAGGTCTAGTAAAAGCCTCTT |
| | | GCACTCTAATGGAAACACCTATTTGTATTGGTACCTGCAGAAGC |
| | | CAGGGCAGTCTCCACAGCTCCTGATCTATCGGATGTCTAATCTCG |
| | | CCTCCGGGGTCCCTGACAGGTTCAGTGGCAGTGGATCAGGCACA |
| | | GATTTTACACTGAAAATCAGCAGAGTGGAGGCTGAGGATGTTGG |
| | | GGTTTATTACTGCATGCAAGGACTACAAACTCCTCTCACTTTCGG |
| | | CGGAGGGACCAAGGTGGAGATCAAA |
| B | 169 | GATATTGTGATGACCCAGTCGCCACTCTCTCTGCCTGTCACCCCT |
| | | GGAGAGCCGGCCTCCATCTCCTGCAAGTCTAGTCAGAGCCTCCT |
| | | CCACTCGGATGGCAAGACCTATTTGAATTGGTACCTGCAGAAGC |
| | | CAGGCCAGTCTCCACAGCTCCTAATCTATTTAGCTTCCCGGCGGG |
| | | CCTCTGGAGTGCCAGATAGGTTCAGTGGCAGCGGGTCAGGGACA |
| | | GATTTCACACTGAAAATCAGCCGGGTGGAGGCTGAGGATGTTGG |
| | | GGTTTATTACTGCTTTCAAGGTACCCACTTTCCTCACACTTTCGG |
| | | CGGAGGGACCAAGGTGGAGATCAAA |
| C | 170 | GATATTGTGATGACTCAGTCTCCACTCTCCCTGCCCGTCACCCCT |
| | | GGAGAGCCGGCCTCCATCTCCTGCAAATCTAGTCAGAGCCTCCT |
| | | GCATTCAGATGGGAAGACATATTTGAACTGGTACCTGCAGAAGC |
| | | CAGGGCAGTCTCCACAGCTCCTGATCTATTTGGGTTCTAATCGGG |
| | | CCTCCGGGGTCCCTGACAGGTTCAGTGGCAGTGGATCAGGCACA |
| | | GATTTTACACTGAAAATCAGCAGAGTGGAGGCTGAGGATGTTGG |
| | | GGTTTATTACTGCTTTCAAGGCACCCATTTCCCTCACACTTTCGG |
| | | CGGAGGGACCAAGGTGGAGATCAAA |
| D | 171 | GATATTGTGATGACCCAGTCACCACTCTCTCTGCCAGTCACCCCT |
| | | GGAGAGCCGGCCTCCATCTCCTGCAAGTCTAGTCAGAGCCTCCT |
| | | ACACTCCGATGGAAAGACCTATTTGAATTGGTACCTGCAGAAGC |
| | | CAGGCCAGTCTCCACAGCTCCTAATCTATTTGGTTTCCAACCGGG |
| | | CCTCTGGAGTGCCAGATAGGTTCAGTGGCAGCGGGTCAGGGACA |
| | | GATTTCACACTGAAAATCAGCCGGGTGGAGGCTGAGGATGTTGG |
| | | GGTTTATTACTGCCACCAAGGTACTCACTTTCCTCATACTTTCGG |
| | | CGGAGGGACCAAGGTGGAGATCAAA |
| E | 172 | GATATTGTGATGACCCAGAGCCCACTCTCTCTGCCCGTCACCCCT |
| | | GGAGAACCGGCCTCCATCTCCTGCAAGTCTAGTCAGAGCCTCCT |
| | | GCACTCCGATGGGAAGACCTATTTGAATTGGTACCTGCAGAAGC |
| | | CAGGCCAGTCTCCACAGCTCCTAATCTATCTGGGCTCCAGCCGG |
| | | GCCTCTGGAGTGCCAGATAGGTTCAGTGGCAGCGGGTCAGGGAC |
| | | AGATTTCACACTGAAAATCAGCCGGGTGGAGGCTGAGGATGTTG |
| | | GGGTTTATTACTGCATGCAAGGTACTCACTTTCCTCATACTTTCG |
| | | GCGGAGGGACCAAGGTGGAGATCAAA |
| F | 173 | GATATTGTGATGACTCAGTCTCCACTCTCCCTGCCCGTCACCCCT |
| | | GGAGAGCCGGCCTCCATCTCCTGCAAGTCTAGTCAGAGCCTCCT |
| | | GCACAATGATGGGAAAACATATTTGAATTGGTACCTGCAGAAGC |
| | | CAGGGCAGTCTCCACAGCTCCTGATCTATTTGGCTTCTAATCGGG |
| | | CCTCCGGGGTCCCTGACAGGTTCAGTGGCAGTGGATCAGGCACA |
| | | GATTTTACACTGAAAATCAGCAGAGTGGAGGCTGAGGATGTTGG |
| | | GGTTTATTACTGCGGCCAAGGCACGCATTTCCCTCATACTTTCGG |
| | | CGGAGGGACCAAGGTGGAGATCAAA |
| G | 174 | GATATTGTGATGACCCAGTCTCCACTCTCTCTGCCGGTCACCCCT |
| | | GGAGAACCGGCCTCCATCTCCTGCAAGTCTAGTCAGAGCCTCCT |
| | | GCACTCTGACGGAAAGACCTATTTGAACTGGTACCTGCAGAAGC |
| | | CAGGCCAGTCTCCACAGCTCCTAATCTATCTGGTTTCCAAGCTGA |
| | | AGTCTGGAGTGCCAGATAGGTTCAGTGGCAGCGGGTCAGGGACA |
| | | GATTTCACACTGAAAATCAGCCGGGTGGAGGCTGAGGATGTTGG |
| | | GGTTTATTACTGCATGCAAGGTACCCACTTTCCTCATACTTTCGG |
| | | CGGAGGGACCAAGGTGGAGATCAAA |
| H | 175 | GATATTGTGATGACCCAGTCCCCACTCTCTCTGCCTGTCACCCCT |
| | | GGAGAGCCGGCCTCCATCTCCTGCAAGTCTAGTCAGAGCCTCCTT |
| | | CACAAGGATGGTAAGACCTATTTGAACTGGTACCTGCAGAAGCC |
| | | AGGCCAGTCTCCACAGCTCCTAATCTATCTGGTTTCCAAACTTCC |
| | | ATCTGGAGTGCCAGATAGGTTCAGTGGCAGCGGGTCAGGGACAG |
| | | ATTTCACACTGAAAATCAGCCGGGTGGAGGCTGAGGATGTTGGG |
| | | GTTTATTACTGCATGCAAGGTACTCACTTCCCTCATACTTTCGGC |
| | | GGAGGGACCAAGGTGGAGATCAAA |
| I | 176 | GATATTGTGATGACTCAGTCTCCACTCTCCCTGCCCGTCACCCCT |
| | | GGAGAGCCGGCCTCCATCTCCTGCAAGTCTAGTCAGAGCCTCTT |
| | | GCACTCTGATGGGAAAACCTATTTGAATTGGTACCTGCAGAAGC |
| | | CAGGGCAGTCTCCACAGCTCCTGATCTATTTGGGTTCTAATCGGG |
| | | CCTCCGGGGTCCCTGACAGGTTCAGTGGCAGTGGATCAGGCACA |
| | | GATTTTACACTGAAAATCAGCAGAGTGGAGGCTGAGGATGTTGG |
| | | GGTTTATTACTGCATGCAAGGAACCCATTTTCCTCACACTTTCGG |
| | | CGGAGGGACCAAGGTGGAGATCAAA |
| J | 177 | GATATCGTGATGACACAGAGCCCCCTGTCGCTGCCCGTGACTCC |
| | | CGGCGAACCCGCAAGCATCAGCTGCAGAAGCTCTCAGAGCATCG |
| | | TGACCGCCATCGGCAACACCTACCTGGAGTGGTATCTGCAGAAG |
| | | CCCGGGCAGAGCCCTCAGCTGCTGATCTACAAGGTGAGCAATAG |
| | | GTTCAGCGGCGTGCCTGATAGATTCAGCGGAAGCGGTAGCGGCA |
| | | CCGACTTCACCCTGAAGATCAGCAGAGTGGAGGCCGAGGACGTG |
| | | GGCGTGTACTATTGCTTCCAAGGCAGCCACGTGCCCTTCACCTTC |
| | | GGCGGCGGCACCAAGGTGGAGATCAAG |
| K | 178 | GATATCGTGATGACACAGAGCCCCCTGAGCCTGCCCGTGACCCC |
| | | CGGCGAGCCCGCAAGCATCAGCTGCAGAAGCAGCAAGAGCCTG |
| | | CTGCACAGCAACGGCAACACCTACCTGTACTGGTATCTGCAGAA |
| | | GCCCGGGCAGAGCCCTCAGCTGCTGATCTACAGAATGAGCAACA |
| | | GAGCAAGCGGCGTGCCCGACAGATTCAGCGGCAGCGGCAGCGG |
| | | CACCGACTTCACCCTGAAGATCAGCAGAGTGGAGGCCGAGGACG |
| | | TGGGCGTCTATTACTGTATGCAAGCCCTGCAGACCCCCTTCACCT |
| | | TCGGCGGCGGCACCAAGGTGGAGATCAAG |
| L | 179 | GATATCGTGATGACACAGAGCCCCCTGAGCCTGCCCGTGACCCC |
| | | CGGCGAACCCGCAAGCATCAGCTGCAAAAGCTCTCAGAGCCTGC |
| | | TGCACAGCGACGGCAAGACCTACCTGTACTGGTATCTGCAGAAG |
| | | CCCGGGCAGAGCCCTCAGCTGCTGATCTACAAGGTGAGCAACAG |
| | | ATTCAAGGGCGTGCCCGACAGATTCAGCGGCAGCGGCAGCGGCA |
| | | CCGACTTCACCCTGAAGATCAGCAGAGTGGAGGCCGAGGACGTG |
| | | GGCGTCTATTACTGTATGCAGAAGATTCAGCTGCCCGTAACATTT |
| | | GGCGGCGGCACCAAGGTGGAGATCAAG |
| M | 180 | GATATCGTGATGACCCAAAGCCCCCTGAGCCTACCCGTGACCCC |
| | | CGGCGAGCCCGCAAGCATCAGCTGCAGAAGCAGCGAGAGCATC |
| | | GTGCACAGCAACGGCAACACCTACCTGGAGTGGTATCTGCAGAA |
| | | GCCCGGGCAGAGCCCTCAGCTGCTGATCTACAAGGTGAGCAACA |
| | | AGTTCAGCGGCGTGCCCGACAGATTCAGCGGCAGCGGCAGCGGC |
| | | ACCGACTTCACCCTGAAGATCAGCAGAGTGGAGGCCGAGGACGT |
| | | GGGCGTCTATTACTGTATGCAGTACACCCACTGGCCCCCCACCTT |
| | | CGGCGGCGGCACCAAGGTGGAGATCAAG |
| N | 181 | GATATCGTGATGACACAGAGCCCCCTGTCGCTGCCCGTGACTCC |
| | | CGGCGAACCCGCAAGCATCAGCTGCAGAAGCTCTCAGAGCCTGC |
| | | TGCACAGCAACGGCTACAACTACCTGGACTGGTATCTGCAGAAG |
| | | CCCGGGCAGAGCCCTCAGCTGCTGATCTACAAGGTGGAGAATAG |
| | | GTTCAGCGGCGTGCCTGATAGATTCAGCGGAAGCGGTAGCGGCA |
| | | CCGACTTCACCCTGAAGATCAGCAGAGTGGAGGCCGAGGACGTG |
| | | GGCGTGTACTATTGTATGCAGCACACCCACTGGCCCCCCACCTTC |
| | | GGCGGCGGCACCAAGGTGGAGATCAAG |
| O | 182 | GATATCGTGATGACACAGAGCCCCCTGTCGCTGCCCGTGACTCC |
| | | CGGCGAACCCGCAAGCATCAGCTGCAGAAGCTCTCAGAGCCTGC |
| | | TGCACAGCAACGGCTACAACTACCTGGACTGGTATCTGCAGAAG |
| | | CCCGGGCAGAGCCCTCAGCTGCTGATCTACAAGGTGAGCAATAG |
| | | GTTCAGCGGCGTGCCTGATAGATTCAGCGGAAGCGGTAGCGGCA |
| | | CCGACTTCACCCTGAAGATCAGCAGAGTGGAGGCCGAGGACGTG |
| | | GGCGTGTACTATTGCCTGCAACACACCCACTGGCCCCCCACCTTC |
| | | GGCGGCGGCACCAAGGTGGAGATCAAG |
| P | 183 | GATATCGTGATGACACAGAGCCCCCTGTCGCTGCCCGTGACTCC |
| | | CGGCGAACCCGCAAGCATCAGCTGCAGAAGCTCTCAGAGCCTGC |
| | | TGCACAGCAACGGCTACAACTACCTGGACTGGTATCTGCAGAAG |
| | | CCCGGGCAGAGCCCTCAGCTGCTGATCTACGAGGTGAGCAATAG |
| | | GTTCAGCGGCGTGCCTGATAGATTCAGCGGAAGCGGTAGCGGCA |
| | | CCGACTTCACCCTGAAGATCAGCAGAGTGGAGGCCGAGGACGTG |
| | | GGCGTGTACTATTGTATGCAGCACACCCACTGGCCCCCCACCTTC |
| | | GGCGGCGGCACCAAGGTGGAGATCAAG |
| Q | 184 | GATATCGTGATGACACAGAGCCCCCTGAGCCTGCCCGTGACCCC |
| | | CGGCGAGCCCGCAAGCATCAGCTGCAGAAGCAGCAGAAGCCTG |
| | | CTGCACAGCAACGGCTACAACTACCTGGACTGGTATCTGCAGAA |
| | | GCCCGGGCAGAGCCCTCAGCTGCTGATCTACGCCGCAAGCAGCC |
| | | TGCAGTCGGGGGTGCCCGACAGATTCAGCGGCAGCGGCAGCGGC |
| | | ACCGACTTCACCCTGAAGATCAGCAGAGTGGAGGCCGAGGACGT |
| | | GGGCGTCTATTACTGCCTGCAACACACCCACTGGCCCCCCACCTT |
| | | CGGCGGCGGCACCAAGGTGGAGATCAAG |
| R | 185 | GATATCGTGATGACACAGAGCCCCCTGTCGCTGCCCGTGACTCC |
| | | CGGCGAACCCGCAAGCATCAGCTGCAGAAGCTCTCAGAGCCTGC |
| | | TGCACAGCAACGGCTACAACTACCTGGACTGGTATCTGCAGAAG |
| | | CCCGGGCAGAGCCCTCAGCTGCTGATCTACAAGGTGAGCAATAG |
| | | GTTCAGCGGCGTGCCTGATAGATTCAGCGGAAGCGGTAGCGGCA |
| | | CCGACTTCACCCTGAAGATCAGCAGAGTGGAGGCCGAGGACGTG |
| | | GGCGTGTACTATTGTATGCAGCACACCCACTGGCCCCCCACCTTC |
| | | GGCGGCGGCACCAAGGTGGAGATCAAG |
| S | 186 | GATATCGTGATGACCCAAAGCCCCCTGAGCCTACCCGTGACCCC |
| | | CGGCGAGCCCGCAAGCATCAGCTGCAGAAGCTCTCAGAGCCTGC |
| | | TGCACAGCAACGGCTACAACTACCTGGACTGGTATCTGCAGAAG |
| | | CCCGGGCAGAGCCCTCAGCTGCTGATCTACAAGGTGAGCAACAG |
| | | ATTCACCGGCGTGCCCGACAGATTCAGCGGCAGCGGCAGCGGCA |
| | | CCGACTTCACCCTGAAGATCAGCAGAGTGGAGGCCGAGGACGTG |
| | | GGCGTCTATTACTGTATGCAGCACACCCACTGGCCCCCCGCCTTC |
| | | GGCGGCGGCACCAAGGTGGAGATCAAG |
| T | 187 | GATATCGTGATGACACAGAGCCCCCTGTCGCTGCCCGTGACTCC |
| | | CGGCGAACCCGCAAGCATCAGCTGCAGAAGCTCTCAGAGCCTGC |
| | | TGCACAGCAACGGCTACAACTACCTGGACTGGTATCTGCAGAAG |
| | | CCCGGGCAGAGCCCTCAGCTGCTGATCTACAAGAGCAGCAATAG |
| | | GTTCAGCGGCGTGCCTGATAGATTCAGCGGAAGCGGTAGCGGCA |
| | | CCGACTTCACCCTGAAGATCAGCAGAGTGGAGGCCGAGGACGTG |
| | | GGCGTGTACTATTGTATGCAGCACACCCACTGGCCCCCCACCTTC |
| | | GGCGGCGGCACCAAGGTGGAGATCAAG |
| U | 188 | GATATCGTGATGACACAGAGCCCCCTGTCGCTGCCCGTGACTCC |
| | | CGGCGAACCCGCAAGCATCAGCTGCAGAAGCTCTCAGAGCCTGC |
| | | TGAACAGCAACGGCGTGACCTTCGTGGAGTGGTATCTGCAGAAG |
| | | CCCGGGCAGAGCCCTCAGCTGCTGATCTACGAGGTGAGCAATAG |
| | | GTTCAGCGGCGTGCCTGATAGATTCAGCGGAAGCGGTAGCGGCA |
| | | CCGACTTCACCCTGAAGATCAGCAGAGTGGAGGCCGAGGACGTG |
| | | GGCGTGTACTATTGCCTGCAACACACCCACTGGCCCCCCACCTTC |
| | | GGCGGCGGCACCAAGGTGGAGATCAAG |
| V | 189 | GATATCGTGATGACCCAAAGCCCCCTGAGCCTACCCGTGACCCC |
| | | CGGCGAGCCCGCAAGCATCAGCTGCAGAAGCTCTCAGAGCCTGC |
| | | TGCACAGCAACGGCTACAACTACCTGGACTGGTATCTGCAGAAG |
| | | CCCGGGCAGAGCCCTCAGCTGCTGATCTACGAGGTGAGCAACAG |
| | | ATTCTGGGGCGTGCCCGACAGATTCAGCGGCAGCGGCAGCGGCA |
| | | CCGACTTCACCCTGAAGATCAGCAGAGTGGAGGCCGAGGACGTG |
| | | GGCGTCTATTACTGCCTGCAACACACCCACTGGCCCCCCACCTTC |
| | | GGCGGCGGCACCAAGGTGGAGATCAAG |
| W | 190 | GATATCGTGATGACACAGAGCCCCCTGAGCCTGCCCGTGACCCC |
| | | CGGCGAGCCCGCAAGCATCAGCTGCAGAAGCAGCAAGAGCCTG |
| | | CTGCACAGCAACGGCAACACCTACCTGTACTGGTATCTGCAGAA |
| | | GCCCGGGCAGAGCCCTCAGCTGCTGATCTACAAGGTGAGCAATC |
| | | AGAGCAGCGGCGTGCCCGACAGATTCAGCGGCAGCGGCAGCGG |
| | | CACCGACTTCACCCTGAAGATCAGCAGAGTGGAGGCCGAGGACG |
| | | TGGGCGTCTATTACTGTATGCAAGCCCTGCAGACCCCCCTGACCT |
| | | TCGGCGGCGGCACCAAGGTGGAGATCAAG |
| X | 191 | GATATCGTGATGACACAGAGCCCCCTGTCGCTGCCCGTGACTCC |
| | | CGGCGAACCCGCAAGCATCAGCTGCAGAAGCTCTCAGAGCCTGC |
| | | TGCACAGCAACGGCTACAACTACCTGGACTGGTATCTGCAGAAG |
| | | CCCGGGCAGAGCCCTCAGCTGCTGATCTACGAGGTGAGCAATAG |
| | | GTTCAGCGGCGTGCCTGATAGATTCAGCGGAAGCGGTAGCGGCA |
| | | CCGACTTCACCCTGAAGATCAGCAGAGTGGAGGCCGAGGACGTG |
| | | GGCGTGTACTATTGCCTGCAACACACCCACTGGCCCCCCACCTTC |
| | | GGCGGCGGCACCAAGGTGGAGATCAAG |
| | SEQ ID | |
| Construct | NO: | scFv Sequence |
| A | 192 | CAGGTGCAGCTGGTGCAGTCTGGGGCTGAGGTGAAGAAGCCTGG |
| | | GGCCTCAGTGAAGGTTTCCTGCAAGGCATCTGGATACGCATTCA |
| | | CCGGGAAGTATATCCACTGGGTGCGACAGGCCCCTGGACAAGGG |
| | | CTTGAGTGGATGGGAGGAATCAACCCAAATAACGGTGGCACAA |
| | | GCTACAATCAGAAGTTCCAGGGCAGAGTCACCATGACCAGGGAC |
| | | ACGTCCACGAGCACAGTCTACATGGAGCTGAGCAGCCTGAGATC |
| | | TGAGGACACGGCCGTGTATTACTGTGCTAGAAAATCCTGGGGCC |
| | | AAGGAACCCTGGTCACCGTCTCCTCAGGTGGCGGAGGATCTGGC |
| | | GGAGGTGGAAGCGGCGGAGGCGGATCTGATATTGTGATGACTCA |
| | | GTCTCCACTCTCCCTGCCCGTCACCCCTGGAGAGCCGGCCTCCAT |
| | | CTCCTGCAGGTCTAGTAAAAGCCTCTTGCACTCTAATGGAAACA |
| | | CCTATTTGTATTGGTACCTGCAGAAGCCAGGGCAGTCTCCACAG |
| | | CTCCTGATCTATCGGATGTCTAATCTCGCCTCCGGGGTCCCTGAC |
| | | AGGTTCAGTGGCAGTGGATCAGGCACAGATTTTACACTGAAAAT |
| | | CAGCAGAGTGGAGGCTGAGGATGTTGGGGTTTATTACTGCATGC |
| | | AAGGACTACAAACTCCTCTCACTTTCGGCGGAGGGACCAAGGTG |
| | | GAGATCAAA |
| B | 193 | CAGGTGCAGCTGGTGCAGTCTGGGGCTGAGGTGAAGAAGCCTGG |
| | | GGCCTCAGTGAAGGTTTCCTGCAAGGCATCTGGATTTACCTTCAC |
| | | CGAGCTGTCCATGCACTGGGTGCGACAGGCCCCTGGACAAGGGC |
| | | TTGAGTGGATGGGAGGAATCAACCCTAATAACGGTGGCACAAGC |
| | | TACAATCAGAAGTTCCAGGGCAGAGTCACCATGACCAGGGACAC |
| | | GTCCACGAGCACAGTCTACATGGAGCTGAGCAGCCTGAGATCTG |
| | | AGGACACGGCCGTGTATTACTGTGCTAGAGAATCCTGGGGCCAA |
| | | GGAACCCTGGTCACCGTCTCCTCAGGTGGCGGAGGATCTGGCGG |
| | | AGGTGGAAGCGGCGGAGGCGGATCTGATATTGTGATGACCCAGT |
| | | CGCCACTCTCTCTGCCTGTCACCCCTGGAGAGCCGGCCTCCATCT |
| | | CCTGCAAGTCTAGTCAGAGCCTCCTCCACTCGGATGGCAAGACC |
| | | TATTTGAATTGGTACCTGCAGAAGCCAGGCCAGTCTCCACAGCT |
| | | CCTAATCTATTTAGCTTCCCGGCGGGCCTCTGGAGTGCCAGATAG |
| | | GTTCAGTGGCAGCGGGTCAGGGACAGATTTCACACTGAAAATCA |
| | | GCCGGGTGGAGGCTGAGGATGTTGGGGTTTATTACTGCTTTCAA |
| | | GGTACCCACTTTCCTCACACTTTCGGCGGAGGGACCAAGGTGGA |
| | | GATCAAA |
| C | 194 | CAGGTGCAGCTGGTGCAGTCTGGGGCTGAGGTGAAGAAGCCTGG |
| | | GGCCTCAGTGAAGGTTTCCTGCAAGGCATCTGGATTCACCTTCAC |
| | | CGAGTACAGTATGCACTGGGTGCGACAGGCCCCTGGACAAGGGC |
| | | TTGAGTGGATGGGAGGCATCAACCCTAACAACGGTGGCACAAGC |
| | | TACAACCAGAAGTTCCAGGGCAGAGTCACCATGACCAGGGACAC |
| | | GTCCACGAGCACAGTCTACATGGAGCTGAGCAGCCTGAGATCTG |
| | | AGGACACGGCCGTGTATTACTGTGCTAGAGAGTCCTGGGGCCAA |
| | | GGAACCCTGGTCACCGTCTCCTCAGGTGGCGGAGGATCTGGCGG |
| | | AGGTGGAAGCGGCGGAGGCGGATCTGATATTGTGATGACTCAGT |
| | | CTCCACTCTCCCTGCCCGTCACCCCTGGAGAGCCGGCCTCCATCT |
| | | CCTGCAAATCTAGTCAGAGCCTCCTGCATTCAGATGGGAAGACA |
| | | TATTTGAACTGGTACCTGCAGAAGCCAGGGCAGTCTCCACAGCT |
| | | CCTGATCTATTTGGGTTCTAATCGGGCCTCCGGGGTCCCTGACAG |
| | | GTTCAGTGGCAGTGGATCAGGCACAGATTTTACACTGAAAATCA |
| | | GCAGAGTGGAGGCTGAGGATGTTGGGGTTTATTACTGCTTTCAA |
| | | GGCACCCATTTCCCTCACACTTTCGGCGGAGGGACCAAGGTGGA |
| | | GATCAAA |
| D | 195 | CAGGTGCAGCTGGTGCAGTCTGGGGCTGAGGTGAAGAAGCCTGG |
| | | GGCCTCAGTGAAGGTTTCCTGCAAGGCATCTGGAGGCACCTTCA |
| | | CCGAGTACTCTATGCACTGGGTGCGACAGGCCCCTGGACAAGGG |
| | | CTTGAGTGGATGGGAGGAATCAACCCCAATAACGGTGGCACACG |
| | | CTACAACCAGAAGTTCCAGGGCAGAGTCACCATGACCAGGGACA |
| | | CGTCCACGAGCACAGTCTACATGGAGCTGAGCAGCCTGAGATCT |
| | | GAGGACACGGCCGTGTATTACTGTGCTAGAGAATCCTGGGGCCA |
| | | AGGAACCCTGGTCACCGTCTCCTCAGGTGGCGGAGGATCTGGCG |
| | | GAGGTGGAAGCGGCGGAGGCGGATCTGATATTGTGATGACCCAG |
| | | TCACCACTCTCTCTGCCAGTCACCCCTGGAGAGCCGGCCTCCATC |
| | | TCCTGCAAGTCTAGTCAGAGCCTCCTACACTCCGATGGAAAGAC |
| | | CTATTTGAATTGGTACCTGCAGAAGCCAGGCCAGTCTCCACAGC |
| | | TCCTAATCTATTTGGTTTCCAACCGGGCCTCTGGAGTGCCAGATA |
| | | GGTTCAGTGGCAGCGGGTCAGGGACAGATTTCACACTGAAAATC |
| | | AGCCGGGTGGAGGCTGAGGATGTTGGGGTTTATTACTGCCACCA |
| | | AGGTACTCACTTTCCTCATACTTTCGGCGGAGGGACCAAGGTGG |
| | | AGATCAAA |
| E | 196 | CAGGTGCAGCTGGTGCAGTCTGGGGCTGAGGTGAAGAAGCCTGG |
| | | GGCCTCAGTGAAGGTTTCCTGCAAGGCATCTGGATTCACCTTCAC |
| | | CGAGTACTCCATGCACTGGGTGCGACAGGCCCCTGGACAAGGGC |
| | | TTGAGTGGATGGGAGGAATCAACCCAAACAACGGTGGCACAAG |
| | | CTACAACCAGAAGTTCCAGGGCAGAGTCACCATGACCAGGGACA |
| | | CGTCCACGAGCACAGTCTACATGGAGCTGAGCAGCCTGAGATCT |
| | | GAGGACACGGCCGTGTATTACTGTGCTAGAGAATCCTGGGGCCA |
| | | AGGAACCCTGGTCACCGTCTCCTCAGGTGGCGGAGGATCTGGCG |
| | | GAGGTGGAAGCGGCGGAGGCGGATCTGATATTGTGATGACCCAG |
| | | AGCCCACTCTCTCTGCCCGTCACCCCTGGAGAACCGGCCTCCATC |
| | | TCCTGCAAGTCTAGTCAGAGCCTCCTGCACTCCGATGGGAAGAC |
| | | CTATTTGAATTGGTACCTGCAGAAGCCAGGCCAGTCTCCACAGC |
| | | TCCTAATCTATCTGGGCTCCAGCCGGGCCTCTGGAGTGCCAGATA |
| | | GGTTCAGTGGCAGCGGGTCAGGGACAGATTTCACACTGAAAATC |
| | | AGCCGGGTGGAGGCTGAGGATGTTGGGGTTTATTACTGCATGCA |
| | | AGGTACTCACTTTCCTCATACTTTCGGCGGAGGGACCAAGGTGG |
| | | AGATCAAA |
| F | 197 | CAGGTGCAGCTGGTGCAGTCTGGGGCTGAGGTGAAGAAGCCTGG |
| | | GGCCTCAGTGAAGGTTTCCTGCAAGGCATCTGGATTCACCTTCAC |
| | | CGAATACAGCATGCACTGGGTGCGACAGGCCCCTGGACAAGGGC |
| | | TTGAGTGGATGGGAGGAATCAACCCCAATAATGGTGGAACAAGC |
| | | TACAATCAGAAGTTCCAGGGCAGAGTCACCATGACCAGGGACAC |
| | | GTCCACGAGCACAGTCTACATGGAGCTGAGCAGCCTGAGATCTG |
| | | AGGACACGGCCGTGTATTACTGTGCTAGAGAATCCTGGGGCCAA |
| | | GGAACCCTGGTCACCGTCTCCTCAGGTGGCGGAGGATCTGGCGG |
| | | AGGTGGAAGCGGCGGAGGCGGATCTGATATTGTGATGACTCAGT |
| | | CTCCACTCTCCCTGCCCGTCACCCCTGGAGAGCCGGCCTCCATCT |
| | | CCTGCAAGTCTAGTCAGAGCCTCCTGCACAATGATGGGAAAACA |
| | | TATTTGAATTGGTACCTGCAGAAGCCAGGGCAGTCTCCACAGCT |
| | | CCTGATCTATTTGGCTTCTAATCGGGCCTCCGGGGTCCCTGACAG |
| | | GTTCAGTGGCAGTGGATCAGGCACAGATTTTACACTGAAAATCA |
| | | GCAGAGTGGAGGCTGAGGATGTTGGGGTTTATTACTGCGGCCAA |
| | | GGCACGCATTTCCCTCATACTTTCGGCGGAGGGACCAAGGTGGA |
| | | GATCAAA |
| G | 198 | CAGGTGCAGCTGGTGCAGTCTGGGGCTGAGGTGAAGAAGCCTGG |
| | | GGCCTCAGTGAAGGTTTCCTGCAAGGCATCTGGATTCACCTTCAC |
| | | CGAGTACTCAATGCACTGGGTGCGACAGGCCCCTGGACAAGGGC |
| | | TTGAGTGGATGGGAGGGATCAACCCAAACAACGGTGGAACAAG |
| | | CTACAATCAGAAGTTCCAGGGCAGAGTCACCATGACCAGGGACA |
| | | CGTCCACGAGCACAGTCTACATGGAGCTGAGCAGCCTGAGATCT |
| | | GAGGACACGGCCGTGTATTACTGTGCTTCCGAGTCCTGGGGCCA |
| | | AGGAACCCTGGTCACCGTCTCCTCAGGTGGCGGAGGATCTGGCG |
| | | GAGGTGGAAGCGGCGGAGGCGGATCTGATATTGTGATGACCCAG |
| | | TCTCCACTCTCTCTGCCGGTCACCCCTGGAGAACCGGCCTCCATC |
| | | TCCTGCAAGTCTAGTCAGAGCCTCCTGCACTCTGACGGAAAGAC |
| | | CTATTTGAACTGGTACCTGCAGAAGCCAGGCCAGTCTCCACAGC |
| | | TCCTAATCTATCTGGTTTCCAAGCTGAAGTCTGGAGTGCCAGATA |
| | | GGTTCAGTGGCAGCGGGTCAGGGACAGATTTCACACTGAAAATC |
| | | AGCCGGGTGGAGGCTGAGGATGTTGGGGTTTATTACTGCATGCA |
| | | AGGTACCCACTTTCCTCATACTTTCGGCGGAGGGACCAAGGTGG |
| | | AGATCAAA |
| H | 199 | CAGGTGCAGCTGGTGCAGTCTGGGGCTGAGGTGAAGAAGCCTGG |
| | | GGCCTCAGTGAAGGTTTCCTGCAAGGCATCTGGATTCACCTTCAC |
| | | CGAGTACTCCATGCACTGGGTGCGACAGGCCCCTGGACAAGGGC |
| | | TTGAGTGGATGGGAGGAATCAACCCAAACAATGGTGGAACAAG |
| | | GTACAACCAGAAGTTCCAGGGCAGAGTCACCATGACCAGGGAC |
| | | ACGTCCACGAGCACAGTCTACATGGAGCTGAGCAGCCTGAGATC |
| | | TGAGGACACGGCCGTGTATTACTGTGCTAGAGAATCCTGGGGCC |
| | | AAGGAACCCTGGTCACCGTCTCCTCAGGTGGCGGAGGATCTGGC |
| | | GGAGGTGGAAGCGGCGGAGGCGGATCTGATATTGTGATGACCCA |
| | | GTCCCCACTCTCTCTGCCTGTCACCCCTGGAGAGCCGGCCTCCAT |
| | | CTCCTGCAAGTCTAGTCAGAGCCTCCTTCACAAGGATGGTAAGA |
| | | CCTATTTGAACTGGTACCTGCAGAAGCCAGGCCAGTCTCCACAG |
| | | CTCCTAATCTATCTGGTTTCCAAACTTCCATCTGGAGTGCCAGAT |
| | | AGGTTCAGTGGCAGCGGGTCAGGGACAGATTTCACACTGAAAAT |
| | | CAGCCGGGTGGAGGCTGAGGATGTTGGGGTTTATTACTGCATGC |
| | | AAGGTACTCACTTCCCTCATACTTTCGGCGGAGGGACCAAGGTG |
| | | GAGATCAAA |
| I | 200 | CAGGTGCAGCTGGTGCAGTCTGGGGCTGAGGTGAAGAAGCCTGG |
| | | GGCCTCAGTGAAGGTTTCCTGCAAGGCATCTGGATTTACCTTCAC |
| | | CGAGTACTCGATGCACTGGGTGCGACAGGCCCCTGGACAAGGGC |
| | | TTGAGTGGATGGGAGGCATCAACCCAAACAACGGTGGCACAATC |
| | | TACAACCAGAAGTTCCAGGGCAGAGTCACCATGACCAGGGACAC |
| | | GTCCACGAGCACAGTCTACATGGAGCTGAGCAGCCTGAGATCTG |
| | | AGGACACGGCCGTGTATTACTGTGCTAGAGAGTCCTGGGGCCAA |
| | | GGAACCCTGGTCACCGTCTCCTCAGGTGGCGGAGGATCTGGCGG |
| | | AGGTGGAAGCGGCGGAGGCGGATCTGATATTGTGATGACTCAGT |
| | | CTCCACTCTCCCTGCCCGTCACCCCTGGAGAGCCGGCCTCCATCT |
| | | CCTGCAAGTCTAGTCAGAGCCTCTTGCACTCTGATGGGAAAACC |
| | | TATTTGAATTGGTACCTGCAGAAGCCAGGGCAGTCTCCACAGCT |
| | | CCTGATCTATTTGGGTTCTAATCGGGCCTCCGGGGTCCCTGACAG |
| | | GTTCAGTGGCAGTGGATCAGGCACAGATTTTACACTGAAAATCA |
| | | GCAGAGTGGAGGCTGAGGATGTTGGGGTTTATTACTGCATGCAA |
| | | GGAACCCATTTTCCTCACACTTTCGGCGGAGGGACCAAGGTGGA |
| | | GATCAAA |
| J | 201 | CAAGTGCAGCTGGTGCAGAGCGGCGCCGAGGTGAAGAAGCCCG |
| | | GCGCAAGCGTGAAGGTGAGCTGCAAGGCAAGCGGCGGCACCTT |
| | | CAGCAGCTACGCCATCAGCTGGGTGAGACAAGCCCCCGGCCAAG |
| | | GCCTGGAGTGGATGGGCGAGATCAACCCTAGCAACGGCAGAAC |
| | | CGTGTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCGCTAGATACAAGGATT |
| | | ATTGGGGGCAAGGCACCTTGGTTACCGTTAGCAGCGGGGGAGGG |
| | | GGCTCGGGGGGGGGAGGCTCGGGCGGTGGCGGAAGTGATATCG |
| | | TGATGACACAGAGCCCCCTGTCGCTGCCCGTGACTCCCGGCGAA |
| | | CCCGCAAGCATCAGCTGCAGAAGCTCTCAGAGCATCGTGACCGC |
| | | CATCGGCAACACCTACCTGGAGTGGTATCTGCAGAAGCCCGGGC |
| | | AGAGCCCTCAGCTGCTGATCTACAAGGTGAGCAATAGGTTCAGC |
| | | GGCGTGCCTGATAGATTCAGCGGAAGCGGTAGCGGCACCGACTT |
| | | CACCCTGAAGATCAGCAGAGTGGAGGCCGAGGACGTGGGCGTG |
| | | TACTATTGCTTCCAAGGCAGCCACGTGCCCTTCACCTTCGGCGGC |
| | | GGCACCAAGGTGGAGATCAAG |
| K | 202 | CAAGTGCAGCTGGTGCAGAGCGGCGCCGAGGTGAAGAAGCCCG |
| | | GCGCAAGCGTGAAGGTGAGCTGCAAGGCAAGCGGCGGCACCTT |
| | | CAGCAGCTACGCCATCCACTGGGTGAGACAAGCCCCCGGCCAAG |
| | | GCCTGGAGTGGATGGGCGAGATCAACCCTAGCAACGGCAGAAC |
| | | CAACTACAATCAGAAGTTCCAAGGCAGAGTGACCATGACAAGA |
| | | GACACAAGCACAAGCACCGTGTACATGGAACTGAGCAGCCTGA |
| | | GAAGCGAGGACACCGCCGTATATTACTGCGCTAGAAGAATGGAC |
| | | TATTGGGGCCAAGGCACCCTGGTAACAGTATCCTCCGGAGGAGG |
| | | AGGCAGCGGCGGGGGGGGCAGTGGCGGGGGAGGGTCCGATATC |
| | | GTGATGACACAGAGCCCCCTGAGCCTGCCCGTGACCCCCGGCGA |
| | | GCCCGCAAGCATCAGCTGCAGAAGCAGCAAGAGCCTGCTGCACA |
| | | GCAACGGCAACACCTACCTGTACTGGTATCTGCAGAAGCCCGGG |
| | | CAGAGCCCTCAGCTGCTGATCTACAGAATGAGCAACAGAGCAAG |
| | | CGGCGTGCCCGACAGATTCAGCGGCAGCGGCAGCGGCACCGACT |
| | | TCACCCTGAAGATCAGCAGAGTGGAGGCCGAGGACGTGGGCGTC |
| | | TATTACTGTATGCAAGCCCTGCAGACCCCCTTCACCTTCGGCGGC |
| | | GGCACCAAGGTGGAGATCAAG |
| L | 203 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGGTGGGCAGCATGAACCCCAACAGCGGCCACACCG |
| | | GCTTCGCTCAGAAGTTCCAAGGCAGAGTGACCATGACAAGAGAC |
| | | ACAAGCACAAGCACCGTGTACATGGAACTGAGCAGCCTGAGAA |
| | | GCGAGGACACCGCCGTATATTACTGCGCTAGAGCTATGGACTAT |
| | | TGGGGGCAAGGCACCCTGGTGACCGTGTCATCCGGAGGTGGGGG |
| | | AAGCGGCGGGGGAGGCTCCGGCGGAGGTGGCAGCGATATCGTG |
| | | ATGACACAGAGCCCCCTGAGCCTGCCCGTGACCCCCGGCGAACC |
| | | CGCAAGCATCAGCTGCAAAAGCTCTCAGAGCCTGCTGCACAGCG |
| | | ACGGCAAGACCTACCTGTACTGGTATCTGCAGAAGCCCGGGCAG |
| | | AGCCCTCAGCTGCTGATCTACAAGGTGAGCAACAGATTCAAGGG |
| | | CGTGCCCGACAGATTCAGCGGCAGCGGCAGCGGCACCGACTTCA |
| | | CCCTGAAGATCAGCAGAGTGGAGGCCGAGGACGTGGGCGTCTAT |
| | | TACTGTATGCAGAAGATTCAGCTGCCCGTAACATTTGGCGGCGG |
| | | CACCAAGGTGGAGATCAAG |
| M | 204 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTATATTACTGCTGGAGATACATGGACT |
| | | ATTGGGGCCAAGGCACCCTGGTAACAGTATCCTCCGGAGGAGGA |
| | | GGCAGCGGCGGGGGGGGCAGTGGCGGGGGAGGGTCCGATATCG |
| | | TGATGACCCAAAGCCCCCTGAGCCTACCCGTGACCCCCGGCGAG |
| | | CCCGCAAGCATCAGCTGCAGAAGCAGCGAGAGCATCGTGCACA |
| | | GCAACGGCAACACCTACCTGGAGTGGTATCTGCAGAAGCCCGGG |
| | | CAGAGCCCTCAGCTGCTGATCTACAAGGTGAGCAACAAGTTCAG |
| | | CGGCGTGCCCGACAGATTCAGCGGCAGCGGCAGCGGCACCGACT |
| | | TCACCCTGAAGATCAGCAGAGTGGAGGCCGAGGACGTGGGCGTC |
| | | TATTACTGTATGCAGTACACCCACTGGCCCCCCACCTTCGGCGGC |
| | | GGCACCAAGGTGGAGATCAAG |
| N | 205 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGCGGGGGAGGG |
| | | GGCTCGGGGGGGGGAGGCTCGGGCGGTGGCGGAAGTGATATCG |
| | | TGATGACACAGAGCCCCCTGTCGCTGCCCGTGACTCCCGGCGAA |
| | | CCCGCAAGCATCAGCTGCAGAAGCTCTCAGAGCCTGCTGCACAG |
| | | CAACGGCTACAACTACCTGGACTGGTATCTGCAGAAGCCCGGGC |
| | | AGAGCCCTCAGCTGCTGATCTACAAGGTGGAGAATAGGTTCAGC |
| | | GGCGTGCCTGATAGATTCAGCGGAAGCGGTAGCGGCACCGACTT |
| | | CACCCTGAAGATCAGCAGAGTGGAGGCCGAGGACGTGGGCGTG |
| | | TACTATTGTATGCAGCACACCCACTGGCCCCCCACCTTCGGCGGC |
| | | GGCACCAAGGTGGAGATCAAG |
| O | 206 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCCAAGAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGCGGGGGAGGG |
| | | GGCTCGGGGGGGGGAGGCTCGGGCGGTGGCGGAAGTGATATCG |
| | | TGATGACACAGAGCCCCCTGTCGCTGCCCGTGACTCCCGGCGAA |
| | | CCCGCAAGCATCAGCTGCAGAAGCTCTCAGAGCCTGCTGCACAG |
| | | CAACGGCTACAACTACCTGGACTGGTATCTGCAGAAGCCCGGGC |
| | | AGAGCCCTCAGCTGCTGATCTACAAGGTGAGCAATAGGTTCAGC |
| | | GGCGTGCCTGATAGATTCAGCGGAAGCGGTAGCGGCACCGACTT |
| | | CACCCTGAAGATCAGCAGAGTGGAGGCCGAGGACGTGGGCGTG |
| | | TACTATTGCCTGCAACACACCCACTGGCCCCCCACCTTCGGCGGC |
| | | GGCACCAAGGTGGAGATCAAG |
| P | 207 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGCGGGGGAGGG |
| | | GGCTCGGGGGGGGGAGGCTCGGGCGGTGGCGGAAGTGATATCG |
| | | TGATGACACAGAGCCCCCTGTCGCTGCCCGTGACTCCCGGCGAA |
| | | CCCGCAAGCATCAGCTGCAGAAGCTCTCAGAGCCTGCTGCACAG |
| | | CAACGGCTACAACTACCTGGACTGGTATCTGCAGAAGCCCGGGC |
| | | AGAGCCCTCAGCTGCTGATCTACGAGGTGAGCAATAGGTTCAGC |
| | | GGCGTGCCTGATAGATTCAGCGGAAGCGGTAGCGGCACCGACTT |
| | | CACCCTGAAGATCAGCAGAGTGGAGGCCGAGGACGTGGGCGTG |
| | | TACTATTGTATGCAGCACACCCACTGGCCCCCCACCTTCGGCGGC |
| | | GGCACCAAGGTGGAGATCAAG |
| Q | 208 | CAAGTGCAGCTGGTCCAAAGCGGTGCAGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTCAGAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTATATTACTGCTGGAGATACATGGACT |
| | | ATTGGGGGCAAGGCACCCTGGTGACCGTGTCAAGCGGAGGTGGG |
| | | GGCAGCGGCGGCGGAGGCTCCGGCGGTGGTGGTTCCGATATCGT |
| | | GATGACACAGAGCCCCCTGAGCCTGCCCGTGACCCCCGGCGAGC |
| | | CCGCAAGCATCAGCTGCAGAAGCAGCAGAAGCCTGCTGCACAGC |
| | | AACGGCTACAACTACCTGGACTGGTATCTGCAGAAGCCCGGGCA |
| | | GAGCCCTCAGCTGCTGATCTACGCCGCAAGCAGCCTGCAGTCGG |
| | | GGGTGCCCGACAGATTCAGCGGCAGCGGCAGCGGCACCGACTTC |
| | | ACCCTGAAGATCAGCAGAGTGGAGGCCGAGGACGTGGGCGTCT |
| | | ATTACTGCCTGCAACACACCCACTGGCCCCCCACCTTCGGCGGC |
| | | GGCACCAAGGTGGAGATCAAG |
| R | 209 | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGCGGGGGAGGG |
| | | GGCTCGGGGGGGGGAGGCTCGGGCGGTGGCGGAAGTGATATCG |
| | | TGATGACACAGAGCCCCCTGTCGCTGCCCGTGACTCCCGGCGAA |
| | | CCCGCAAGCATCAGCTGCAGAAGCTCTCAGAGCCTGCTGCACAG |
| | | CAACGGCTACAACTACCTGGACTGGTATCTGCAGAAGCCCGGGC |
| | | AGAGCCCTCAGCTGCTGATCTACAAGGTGAGCAATAGGTTCAGC |
| | | GGCGTGCCTGATAGATTCAGCGGAAGCGGTAGCGGCACCGACTT |
| | | CACCCTGAAGATCAGCAGAGTGGAGGCCGAGGACGTGGGCGTG |
| | | TACTATTGTATGCAGCACACCCACTGGCCCCCCACCTTCGGCGGC |
| | | GGCACCAAGGTGGAGATCAAG |
| S | 210 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACCCTCAGAAGTTCCAAGGCAGAGTGACCATGACAAGAGA |
| | | CACAAGCACAAGCACCGTGTACATGGAACTGAGCAGCCTGAGA |
| | | AGCGAGGACACCGCCGTATATTACTGCTGGAGATACATGGACTA |
| | | TTGGGGCCAAGGCACCCTGGTAACAGTATCCTCCGGAGGAGGAG |
| | | GCAGCGGCGGGGGGGGCAGTGGCGGGGGAGGGTCCGATATCGT |
| | | GATGACCCAAAGCCCCCTGAGCCTACCCGTGACCCCCGGCGAGC |
| | | CCGCAAGCATCAGCTGCAGAAGCTCTCAGAGCCTGCTGCACAGC |
| | | AACGGCTACAACTACCTGGACTGGTATCTGCAGAAGCCCGGGCA |
| | | GAGCCCTCAGCTGCTGATCTACAAGGTGAGCAACAGATTCACCG |
| | | GCGTGCCCGACAGATTCAGCGGCAGCGGCAGCGGCACCGACTTC |
| | | ACCCTGAAGATCAGCAGAGTGGAGGCCGAGGACGTGGGCGTCT |
| | | ATTACTGTATGCAGCACACCCACTGGCCCCCCGCCTTCGGCGGC |
| | | GGCACCAAGGTGGAGATCAAG |
| T | 211 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGCGGGGGAGGG |
| | | GGCTCGGGGGGGGGAGGCTCGGGCGGTGGCGGAAGTGATATCG |
| | | TGATGACACAGAGCCCCCTGTCGCTGCCCGTGACTCCCGGCGAA |
| | | CCCGCAAGCATCAGCTGCAGAAGCTCTCAGAGCCTGCTGCACAG |
| | | CAACGGCTACAACTACCTGGACTGGTATCTGCAGAAGCCCGGGC |
| | | AGAGCCCTCAGCTGCTGATCTACAAGAGCAGCAATAGGTTCAGC |
| | | GGCGTGCCTGATAGATTCAGCGGAAGCGGTAGCGGCACCGACTT |
| | | CACCCTGAAGATCAGCAGAGTGGAGGCCGAGGACGTGGGCGTG |
| | | TACTATTGTATGCAGCACACCCACTGGCCCCCCACCTTCGGCGGC |
| | | GGCACCAAGGTGGAGATCAAG |
| U | 212 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTTTACTACTGCTGGAGATACATGGATT |
| | | ATTGGGGGCAAGGCACCTTGGTTACCGTTAGCAGCGGGGGAGGG |
| | | GGCTCGGGGGGGGGAGGCTCGGGCGGTGGCGGAAGTGATATCG |
| | | TGATGACACAGAGCCCCCTGTCGCTGCCCGTGACTCCCGGCGAA |
| | | CCCGCAAGCATCAGCTGCAGAAGCTCTCAGAGCCTGCTGAACAG |
| | | CAACGGCGTGACCTTCGTGGAGTGGTATCTGCAGAAGCCCGGGC |
| | | AGAGCCCTCAGCTGCTGATCTACGAGGTGAGCAATAGGTTCAGC |
| | | GGCGTGCCTGATAGATTCAGCGGAAGCGGTAGCGGCACCGACTT |
| | | CACCCTGAAGATCAGCAGAGTGGAGGCCGAGGACGTGGGCGTG |
| | | TACTATTGCCTGCAACACACCCACTGGCCCCCCACCTTCGGCGGC |
| | | GGCACCAAGGTGGAGATCAAG |
| V | 213 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGAGATCAACCCTAGCAACGGCAGAACC |
| | | AACTACAATCAGAAGTTCCAAGGCAGAGTGACCATGACAAGAG |
| | | ACACAAGCACAAGCACCGTGTACATGGAACTGAGCAGCCTGAG |
| | | AAGCGAGGACACCGCCGTATATTACTGCTGGAGATACATGGACT |
| | | ATTGGGGCCAAGGCACCCTGGTAACAGTATCCTCCGGAGGAGGA |
| | | GGCAGCGGCGGGGGGGGCAGTGGCGGGGGAGGGTCCGATATCG |
| | | TGATGACCCAAAGCCCCCTGAGCCTACCCGTGACCCCCGGCGAG |
| | | CCCGCAAGCATCAGCTGCAGAAGCTCTCAGAGCCTGCTGCACAG |
| | | CAACGGCTACAACTACCTGGACTGGTATCTGCAGAAGCCCGGGC |
| | | AGAGCCCTCAGCTGCTGATCTACGAGGTGAGCAACAGATTCTGG |
| | | GGCGTGCCCGACAGATTCAGCGGCAGCGGCAGCGGCACCGACTT |
| | | CACCCTGAAGATCAGCAGAGTGGAGGCCGAGGACGTGGGCGTCT |
| | | ATTACTGCCTGCAACACACCCACTGGCCCCCCACCTTCGGCGGC |
| | | GGCACCAAGGTGGAGATCAAG |
| W | 214 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACCAAGTACTGGATGCACTGGGTGAGACAAGCCCCCGGCCAAGG |
| | | CCTGGAGTGGATGGGCGGCATCAACCCTAGCAACGGCAGAACCA |
| | | ACTACAATCAGAAGTTCCAAGGCAGAGTGACCATGACAAGAGA |
| | | CACAAGCACAAGCACCGTGTACATGGAACTGAGCAGCCTGAGA |
| | | AGCGAGGACACCGCCGTATATTACTGCGCTAGATACATGCAGTA |
| | | TTGGGGCCAAGGCACCCTGGTAACAGTATCCTCCGGAGGAGGAG |
| | | GCAGCGGCGGGGGGGGCAGTGGCGGGGGAGGGTCCGATATCGT |
| | | GATGACACAGAGCCCCCTGAGCCTGCCCGTGACCCCCGGCGAGC |
| | | CCGCAAGCATCAGCTGCAGAAGCAGCAAGAGCCTGCTGCACAGC |
| | | AACGGCAACACCTACCTGTACTGGTATCTGCAGAAGCCCGGGCA |
| | | GAGCCCTCAGCTGCTGATCTACAAGGTGAGCAATCAGAGCAGCG |
| | | GCGTGCCCGACAGATTCAGCGGCAGCGGCAGCGGCACCGACTTC |
| | | ACCCTGAAGATCAGCAGAGTGGAGGCCGAGGACGTGGGCGTCT |
| | | ATTACTGTATGCAAGCCCTGCAGACCCCCCTGACCTTCGGCGGC |
| | | GGCACCAAGGTGGAGATCAAG |
| X | 215 | CAAGTGCAGCTGGTGCAGAGCGGCGCCGAGGTGAAGAAGCCCG |
| | | GCGCAAGCGTGAAGGTGAGCTGCAAGGCAAGCGGCTACACCTTC |
| | | ACAAGCCACTACCTGCACTGGGTGAGACAAGCCCCCGGCCAAGG |
| | | CCTGGAGTGGATGGGCATCATCAACCCTAGCAACGGCAGAACCA |
| | | ACTACAATCAGAAGTTCCAAGGCAGAGTGACCATGACAAGAGA |
| | | CACAAGCACAAGCACCGTGTACATGGAACTGAGCAGCCTGAGA |
| | | AGCGAGGACACCGCCGTTTACTACTGCTGGAGATACATGGATTA |
| | | TTGGGGGCAAGGCACCTTGGTTACCGTTAGCAGCGGGGGAGGGG |
| | | GCTCGGGGGGGGGAGGCTCGGGCGGTGGCGGAAGTGATATCGT |
| | | GATGACACAGAGCCCCCTGTCGCTGCCCGTGACTCCCGGCGAAC |
| | | CCGCAAGCATCAGCTGCAGAAGCTCTCAGAGCCTGCTGCACAGC |
| | | AACGGCTACAACTACCTGGACTGGTATCTGCAGAAGCCCGGGCA |
| | | GAGCCCTCAGCTGCTGATCTACGAGGTGAGCAATAGGTTCAGCG |
| | | GCGTGCCTGATAGATTCAGCGGAAGCGGTAGCGGCACCGACTTC |
| | | ACCCTGAAGATCAGCAGAGTGGAGGCCGAGGACGTGGGCGTGT |
| | | ACTATTGCCTGCAACACACCCACTGGCCCCCCACCTTCGGCGGC |
| | | GGCACCAAGGTGGAGATCAAG |

In certain embodiments, a polynucleotide encoding an antibody described herein is isolated or purified.

In particular aspects, provided herein are polynucleotides comprising nucleotide sequences encoding antibodies which specifically bind to a TDP-43 (e.g., human TDP-43) polypeptide and comprise an amino acid sequence as described herein, as well as antibodies which compete with such antibodies for binding to a TDP-43 (e.g., human TDP-43) polypeptide (e.g., in a dose-dependent manner), or which binds to the same epitope as that of such antibodies.

In certain aspects, provided herein are polynucleotides comprising a nucleotide sequence encoding the light chain or heavy chain of antibody described herein. The polynucleotides can comprise nucleotide sequences encoding a light chain comprising the VL FRs and CDRs of antibodies described herein (see, e.g., Table 1) or nucleotide sequences encoding a heavy chain comprising the VH FRs and CDRs of antibodies described herein (see, e.g., Table 1). In certain embodiments, a polynucleotide encodes a VH, VL, heavy chain, and/or light chain of an antibody described herein. In another embodiment, a polynucleotide encodes a VH and a VL of an antibody described herein. In another embodiment, a polynucleotide encodes a peptide linker described herein. In another embodiment, a polynucleotide encodes an scFv described herein. In another embodiment, a polynucleotide encodes the VH and/or the VL, or the heavy chain and/or the light chain, of an antibody described herein.

Also provided herein are polynucleotides encoding an anti-TDP-43 antibody that are optimized, e.g., by codon/RNA optimization, replacement with heterologous signal sequences, and elimination of mRNA instability elements. Methods to generate optimized polynucleotides encoding an anti-TDP-43 antibody or a fragment thereof (e.g., VH and/or VL) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleotide sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of an anti-TDP-43 antibody or fragment thereof by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an anti-TDP-43 antibody encoded by polynucleotides that have not been optimized.

In certain embodiments, an optimized polynucleotide sequence encoding an anti-TDP-43 antibody described herein or a fragment thereof (e.g., VL domain and/or VH domain) can hybridize to an antisense (e.g., complementary) polynucleotide of an unoptimized polynucleotide sequence encoding an anti-TDP-43 antibody described herein or a fragment thereof (e.g., VL domain and/or VH domain). In specific embodiments, an optimized nucleotide sequence encoding an anti-TDP-43 antibody described herein or a fragment hybridizes under high stringency conditions to antisense polynucleotide of an unoptimized polynucleotide sequence encoding an anti-TDP-43 antibody described herein or a fragment thereof. In a specific embodiment, an optimized nucleotide sequence encoding an anti-TDP-43 antibody described herein or a fragment thereof hybridizes under high stringency, intermediate or lower stringency hybridization conditions to an antisense polynucleotide of an unoptimized nucleotide sequence encoding an anti-TDP-43 antibody described herein or a fragment thereof. Information regarding hybridization conditions has been described, see, e.g., U.S. Patent Application Publication No. US 2005/0048549 (e.g., paragraphs 72-73), which is herein incorporated by reference in its entirety.

The polynucleotides can be obtained, and the nucleotide sequence of the polynucleotides determined, by any method known in the art. Nucleotide sequences encoding antibodies described herein, e.g., antibodies described in Table 1, and modified versions of these antibodies can be determined using methods well known in the art, i.e., nucleotide codons known to encode particular amino acids are assembled in such a way to generate a polynucleotide that encodes the antibody. Such a polynucleotide encoding the antibody can be assembled from chemically synthesized oligonucleotides (e.g., as described in Kutmeier G et al., (1994), BioTechniques 17:242-6, herein incorporated by reference in its entirety), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antigen-binding region of a described here or an antibody described herein can be generated from a polynucleotide from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain polynucleotides comprising the nucleotide sequence encoding the light chain and/or heavy chain of an antibody. Such PCR amplification methods can be used to obtain polynucleotides comprising the nucleotide sequence encoding the variable light chain region and/or the variable heavy chain region of an antibody. The amplified polynucleotides can be cloned into vectors for expression in host cells and for further cloning.

If a clone containing a polynucleotide encoding a particular antigen-binding region or antibody is not available, but the sequence of the antigen-binding region or antibody molecule is known, a polynucleotide encoding the immunoglobulin can be chemically synthesized or obtained from a suitable source (e.g., an antibody cDNA library or a cDNA library generated from, or polynucleotide, preferably poly A+ RNA, isolated from, any tissue or cells expressing the antibody, such as hybridoma cells selected to express an antibody described herein) by PCR amplification using synthetic primers hybridizable to the 3′ and 5′ ends of the sequence, or by cloning using an oligonucleotide probe specific for the particular gene sequence to identify, e.g., a cDNA clone from a cDNA library that encodes the antibody. Amplified polynucleotides generated by PCR can then be cloned into replicable cloning vectors using any method well known in the art.

DNA encoding anti-TDP-43 (e.g., human TDP-43) antibodies described herein can be readily isolated and sequenced using conventional procedures (e.g., by using oligonucleotide probes that are capable of binding specifically to genes encoding the heavy and light chains of the anti-TDP-43 (e.g., human TDP-43) antibodies). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-TDP-43 antibodies in the recombinant host cells.

To generate whole antibodies or antigen-binding regions, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 1 or human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1a promoter, a secretion signal, a cloning site for the variable region, constant regions, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, for example, by substituting the coding sequence for human heavy and light chain constant regions in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH domain and/or VL domain provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In certain embodiments, the polynucleotide is comprised within a vector.

In certain embodiments, the vector is a non-viral vector. Exemplary non-viral vectors include, but are not limited to, plasmid DNA, transposons, episomal plasmids, minicircles, ministrings, and oligonucleotides (e.g., mRNA, naked DNA). In an embodiment, the non-viral vector is a transposon-based vector. In an embodiment, the non-viral vector is a PiggyBac-based vector, or a Sleeping Beauty-based vector.

In certain embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV of clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5), retrovirus vectors (e.g., MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, M1), flavivirus (e.g., Kunjin, West Nile, Dengue virus), rhabdovirus vectors (e.g., rabies virus, VSV), measles virus vector, Newcastle disease virus vectors, poxvirus vectors, and picornavirus vectors (e.g., Coxsackievirus). In an embodiment, the viral vector is selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus.

In certain embodiments, the vector targets a cortical neuron, a spinal neuron, and/or an astrocyte.

In certain embodiments, the vector is a recombinant AAV (rAAV), wherein the rAAV comprises an AAV capsid comprising an AAV capsid protein; and a rAAV vector genome.

In certain embodiments, the capsid protein is a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

In certain embodiments, the capsid protein is an engineered variant capsid protein comprising amino acid sequences from at least two different AAV capsid proteins. In an embodiment, the capsid protein comprises amino acid sequences from an AAV2 capsid protein and an AAV5 capsid protein. In an embodiment, the capsid protein comprises a VP1 amino acid sequence from AAV2 and a VP2 and VP3 amino acid sequence from AAV5. In certain embodiments, the engineered variant capsid is AAV5.2.

Exemplary capsid protein sequences are disclosed in Table 4 below.

TABLE 4
Exemplary capsid protein sequences

| Capsid | SEQ | |
|---|---|---|
| protein | ID NO: | Amino acid sequence |
| AAV5 | 216 | MSFVDHPPDWLEEVGEGLREFLGLEAGPPKPKPNQQHQDQARGL |
| | | VLPGYNYLGPGNGLDRGEPVNRADEVAREHDISYNEQLEAGDNP |
| | | YLKYNHADAEFQEKLADDTSFGGNLGKAVFQAKKRVLEPFGLVE |
| | | EGAKTAPTGKRIDDHFPKRKKARTEEDSKPSTSSDAEAGPSGSQQ |
| | | LQIPAQPASSLGADTMSAGGGGPLGDNNQGADGVGNASGDWHC |
| | | DSTWMGDRVVTKSTRTWVLPSYNNHQYREIKSGSVDGSNANAY |
| | | FGYSTPWGYFDFNRFHSHWSPRDWQRLINNYWGFRPRSLRVKIFN |
| | | IQVKEVTVQDSTTTIANNLTSTVQVFTDDDYQLPYVVGNGTEGCL |
| | | PAFPPQVFTLPQYGYATLNRDNTENPTERSSFFCLEYFPSKMLRTG |
| | | NNFEFTYNFEEVPFHSSFAPSQNLFKLANPLVDQYLYRFVSTNNTG |
| | | GVQFNKNLAGRYANTYKNWFPGPMGRTQGWNLGSGVNRASVS |
| | | AFATTNRMELEGASYQVPPQPNGMTNNLQGSNTYALENTMIFNS |
| | | QPANPGTTATYLEGNMLITSESETQPVNRVAYNVGGQMATNNQS |
| | | STTAPATGTYNLQEIVPGSVWMERDVYLQGPIWAKIPETGAHFHP |
| | | SPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYR |
| | | TTRPIGTRYLTRPL |
| AAV5.2 | 217 | MAADGYLPDWLEDTLSEGIRQWWKLKPGPPPPKPAERHKDDSRG |
| | | LVLPGYKYLGPFNGLDKGEPVNEADAAALEHDKAYDRQLDSGD |
| | | NPYLKYNHADAEFQERLKEDTSFGGNLGRAVFQAKKRVLEPLGL |
| | | VEEPVKTAPTGKRIDDHFPKRKKARTEEDSKPSTSSDAEAGPSGSQ |
| | | QLQIPAQPASSLGADTMSAGGGGPLGDNNQGADGVGNASGDWH |
| | | CDSTWMGDRVVTKSTRTWVLPSYNNHQYREIKSGSVDGSNANA |
| | | YFGYSTPWGYFDFNRFHSHWSPRDWQRLINNYWGFRPRSLRVKIF |
| | | NIQVKEVTVQDSTTTIANNLTSTVQVFTDDDYQLPYVVGNGTEGC |
| | | LPAFPPQVFTLPQYGYATLNRDNTENPTERSSFFCLEYFPSKMLRT |
| | | GNNFEFTYNFEEVPFHSSFAPSQNLFKLANPLVDQYLYRFVSTNNT |
| | | GGVQFNKNLAGRYANTYKNWFPGPMGRTQGWNLGSGVNRASV |
| | | SAFATTNRMELEGASYQVPPQPNGMTNNLQGSNTYALENTMIFN |
| | | SQPANPGTTATYLEGNMLITSESETQPVNRVAYNVGGQMATNNQ |
| | | SSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYR |
| | | TTRPIGTRYLTRPL |

In an embodiment, the capsid protein comprises an amino acid sequence that has at least 80%, 81%, 82%, 83%, 84%, 85%, 86%, 87%, 88%, 89%, 90%, 91%, 92%, 93%, 94%, 95%, 96%, 97%, or 98% identity to SEQ ID NO: 216 or 217. In an embodiment, the capsid protein comprises an amino acid sequence that has at least 99% identity to SEQ ID NO: 216 or 217. In an embodiment, the capsid protein comprises the amino acid sequence of SEQ ID NO: 216 or 217.

In an embodiment, the rAAV comprises an AAV capsid comprising an AAV capsid protein, and a rAAV genome comprising a polynucleotide encoding an antibody that specifically binds to TDP-43.

In an embodiment, the rAAV genome further comprises a 5′ inverted terminal repeat (5′ ITR) nucleotide sequence, and a 3′ inverted terminal repeat (3′ ITR) nucleotide sequence. ITR sequences from any AAV serotype or variant thereof can be used in the rAAV genomes disclosed herein. The 5′ and 3′ ITR can be from an AAV of the same serotype or from AAVs of different serotypes.

In certain aspects, provided herein is a packaging system for preparation of an rAAV, wherein the packaging system comprises: (a) a first nucleotide sequence encoding one or more AAV Rep proteins; (b) a second nucleotide sequence encoding a capsid protein of an rAAV described herein; and (c) a third nucleotide sequence comprising an rAAV genome sequence of an rAAV described herein. In an embodiment, the packaging system comprises a first vector comprising the first nucleotide sequence and the second nucleotide sequence, and a second vector comprising the third nucleotide sequence. In an embodiment, the packaging system further comprises a fourth nucleotide sequence comprising one or more helper virus genes. In an embodiment, the fourth nucleotide sequence is comprised within a third vector. In an embodiment, the fourth nucleotide sequence comprises one or more genes from a virus selected from the group consisting of adenovirus, herpes virus, vaccinia virus, and cytomegalovirus (CMV). In an embodiment, the first vector, second vector, and/or the third vector is a plasmid.

In certain aspects, provided herein is a method for recombinant preparation of an rAAV, the method comprising introducing a packaging system described herein into a cell under conditions whereby the rAAV is produced.

In certain aspects, provided herein are cells (e.g., host cells) expressing (e.g., recombinantly) antibodies described herein which specifically bind to TDP-43 (e.g., human TDP-43), and related polynucleotides and expression vectors. Provided herein are vectors (e.g., expression vectors) comprising polynucleotides comprising nucleotide sequences encoding anti-TDP-43 antibodies or a fragment for recombinant expression in host cells, preferably in mammalian cells (e.g., CHO cells). Also provided herein are host cells comprising such vectors for recombinantly expressing anti-TDP-43 antibodies described herein (e.g., human or humanized antibody). In a particular aspect, provided herein are methods for producing an antibody described herein, comprising expressing the antibody from a host cell.

Recombinant expression of an antibody described herein (e.g., a full-length antigen-binding region or antibody or heavy and/or light chain of an antibody described herein) that specifically binds to TDP-43 (e.g., human TDP-43) generally involves construction of an expression vector containing a polynucleotide that encodes the antibody. Once a polynucleotide encoding an antibody molecule, heavy and/or light chain of an antibody, or a fragment thereof (e.g., heavy and/or light chain variable regions) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing an antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding containing an antibody molecule described herein, a heavy or light chain of an antibody, a heavy or light chain variable region of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464, which are herein incorporated by reference in their entireties) and variable regions of the antibody can be cloned into such a vector for expression of the entire heavy, the entire light chain, or both the entire heavy and light chains.

In certain embodiments, a vector comprises a polynucleotide encoding a VH, VL, heavy chain, and/or light chain of an antibody described herein. In another embodiment, a vector comprises a polynucleotide encoding the VH and the VL of an antibody described herein. In another embodiment, a vector comprises a polynucleotide encoding the heavy chain and the light chain of an antibody described herein.

An expression vector can be transferred to a cell (e.g., host cell) by conventional techniques and the resulting cells can then be cultured by conventional techniques to produce an antibody described herein or a fragment thereof. Thus, provided herein are host cells containing a polynucleotide encoding containing an antibody described herein or fragments thereof, or a heavy or light chain thereof, or fragment thereof, or a single-chain antibody described herein, operably linked to a promoter for expression of such sequences in the host cell.

In certain embodiments, a host cell comprises a polynucleotide encoding the VH and VL of an antibody described herein. In another embodiment, a host cell comprises a vector comprising a polynucleotide encoding the VH and VL of an antibody described herein. In another embodiment, a host cell comprises a first polynucleotide encoding the VH of an antibody described herein, and a second polynucleotide encoding the VL of an antibody described herein. In another embodiment, a host cell comprises a first vector comprising a first polynucleotide encoding the VH of an antibody described herein, and a second vector comprising a second polynucleotide encoding the VL of an antibody described herein.

In specific embodiments, a heavy chain/heavy chain variable region expressed by a first cell is associated with a light chain/light chain variable region of a second cell to form an anti-TDP-43 (e.g., human TDP-43) antibody described herein. In certain embodiments, provided herein is a population of host cells comprising such first host cell and such second host cell.

In certain embodiments, provided herein is a population of vectors comprising a first vector comprising a polynucleotide encoding a light chain/light chain variable region of an anti-TDP-43 (e.g., human TDP-43) antibody described herein, and a second vector comprising a polynucleotide encoding a heavy chain/heavy chain variable region of an anti-TDP-43 (e.g., human TDP-43) antibody described herein.

A variety of host-expression vector systems can be utilized to express antibody molecules described herein (see, e.g., U.S. Pat. No. 5,807,715, which is herein incorporated by reference in its entirety). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with, e.g., recombinant bacteriophage DNA, plasmid DNA or cosmid DNA expression vectors containing antibody coding sequences; yeast (e.g., Saccharomyces and Pichia) transformed with, e.g., recombinant yeast expression vectors containing antibody coding sequences; insect cell systems infected with, e.g., recombinant virus expression vectors (e.g., baculovirus) containing antibody coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20 and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies described herein are Chinese hamster ovary (CHO) cells, for example CHO cells from the CHO GS System™ (Lonza). In certain embodiments, the heavy chain and/or light chain of an antibody produced by a CHO cell may have an N-terminal glutamine or glutamate residue replaced by pyroglutamate. In certain embodiments, cells for expressing antibodies described herein are human cells, e.g., human cell lines. In a specific embodiment, a mammalian expression vector is pOptiVEC™ or pcDNA3.3. In certain embodiments, bacterial cells such as Escherichia coli, or eukaryotic cells (e.g., mammalian cells), especially for the expression of whole recombinant antibody molecule, are used for the expression of a recombinant antibody molecule. For example, mammalian cells such as CHO cells, in conjunction with a vector such as the major intermediate early gene promoter element from human cytomegalovirus, are an effective expression system for antibodies (Foecking M K & Hofstetter H (1986) Gene 45:101-5; and Cockett M I et al., (1990) Biotechnology 8 (7): 662-7, each of which is herein incorporated by reference in its entirety). In certain embodiments, antibodies described herein are produced by CHO cells or NS0 cells. In a specific embodiment, the expression of nucleotide sequences encoding antibodies described herein which specifically bind to TDP-43 (e.g., human TDP-43) is regulated by a constitutive promoter, inducible promoter, or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of elevated levels of fusion protein products that are readily purified can be desirable. Such vectors include, but are not limited to, the E. coli expression vector pUR278 (Ruether U & Mueller-Hill B (1983) EMBO J 2:1791-1794), in which the coding sequence can be ligated individually into the vector in frame with the lac Z coding region so that a fusion protein is produced; pIN vectors (Inouye S & Inouye M (1985) Nuc Acids Res 13:3101-3109; Van Heeke G & Schuster S M (1989) J Biol Chem 24:5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the molecule in infected hosts (e.g., see Logan J & Shenk T (1984) PNAS 81 (12): 3655-9, which is herein incorporated by reference in its entirety). Specific initiation signals can also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153:516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20, T47D, NS0 (a murine myeloma cell line that does not endogenously produce any immunoglobulin chains), CRL7030, COS (e.g., COS1 or COS), PER.C6, VERO, HEK-293T, HepG2, SP210, R1.1, B-W, L-M, BSC1, BSC40, YB/20, BMT10, and HsS78Bst cells. In certain embodiments, anti-TDP-43 (e.g., human TDP-43) antibodies described herein are produced in mammalian cells, such as CHO cells.

In a specific embodiment, the antibodies described herein have reduced fucose content or no fucose content. Such antibodies can be produced using techniques known to one skilled in the art. For example, the antibodies can be expressed in cells deficient or lacking the ability of to fucosylate. In a specific example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express an anti-TDP-43 (e.g., human TDP-43) antibody described herein can be engineered. In specific embodiments, a cell provided herein stably expresses a light chain/light chain variable region and a heavy chain/heavy chain variable region which associate to form an antigen-binding region or an antibody described herein.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-TDP-43 (e.g., human TDP-43) described herein or a fragment thereof. Such engineered cell lines can be particularly useful in screening and evaluation of compositions that interact directly or indirectly with the antibody molecule.

A number of selection systems can be used, including but not limited to the herpes simplex virus thymidine kinase (Wigler M et al., (1977) Cell 11 (1): 223-32), hypoxanthineguanine phosphoribosyltransferase (Szybalska E H & Szybalski W (1962) PNAS 48 (12): 2026-2034) and adenine phosphoribosyltransferase (Lowy I et al., (1980) Cell 22 (3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77 (6): 3567-70; O'Hare K et al., (1981) PNAS 78:1527-31); gpt, which confers resistance to mycophenolic acid (Mulligan R C & Berg P (1981) PNAS 78 (4): 2072-6); neo, which confers resistance to the aminoglycoside G-418 (Wu G Y & Wu C H (1991) Biotherapy 3:87-95; Tolstoshev P (1993) Ann Rev Pharmacol Toxicol 32:573-596; Mulligan R C (1993) Science 260:926-932; and Morgan R A & Anderson W F (1993) Ann Rev Biochem 62:191-217; Nabel G J & Felgner P L (1993) Trends Biotechnol 11 (5): 211-5); and hygro, which confers resistance to hygromycin (Santerre R F et al., (1984) Gene 30 (1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150:1-14, all of which are herein incorporated by reference in their entireties.

The expression levels of an antibody molecule can be increased by vector amplification (for a review, see Bebbington C R & Hentschel C C G, The use of vectors based on gene amplification for the expression of cloned genes in mammalian cells in DNA cloning, Vol. 3 (Academic Press, New York, 1987), which is herein incorporated by reference in its entirety). When a marker in the vector system is amplifiable, increase in the level of inhibitor present in culture of host cell will increase the number of copies of the marker gene. Since the amplified region is associated with the gene of interest, production of the protein will also increase (Crouse G F et al., (1983) Mol Cell Biol 3:257-66, which is herein incorporated by reference in its entirety).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: about 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot N J (1986) Nature 322:562-565; and Köhler G (1980) PNAS 77:2197-2199, each of which is herein incorporated by reference in its entirety). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more genes/nucleotide sequences, or in the range of 2-5, 5-10, or 10-20 genes/nucleotide sequences. For example, a bicistronic nucleic acid construct can comprise, in the following order, a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. In certain embodiments, an isolated antibody is one that is substantially free of other antibodies with different antigenic specificities than the isolated antibody. For example, in certain embodiments, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly, such preparations of the antibody have less than about 30%, 20%, 10%, or 5% (by dry weight) of chemical precursors or compounds other than the antibody of interest. In a specific embodiment, antibodies described herein are isolated or purified.

Anti-TDP-43 (e.g., human TDP-43) antibodies or fragments thereof can be produced by any method known in the art for the synthesis of proteins or antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press, all of which are herein incorporated by reference in their entireties.

In a specific embodiment, an antibody described herein is prepared, expressed, created, or isolated by any means that involves creation, e.g., via synthesis, genetic engineering of DNA sequences. In certain embodiments, such an antibody comprises sequences (e.g., DNA sequences or amino acid sequences) that do not naturally exist within the antibody germline repertoire of an animal or mammal (e.g., human) in vivo.

In one aspect, provided herein is a method of making an anti-TDP-43 (e.g., human TDP-43) antibody comprising culturing a cell or host cell described herein. In certain embodiments, the method is performed in vitro. In a certain aspect, provided herein is a method of making an anti-TDP-43 (e.g., human TDP-43) antibody comprising expressing (e.g., recombinantly expressing) the antibody using a cell or host cell described herein (e.g., a cell or a host cell comprising polynucleotides encoding an antibody described herein). In certain embodiments, the cell is an isolated cell. In certain embodiments, the exogenous polynucleotides have been introduced into the cell. In certain embodiments, the method further comprises the step of purifying the antibody obtained from the cell or host cell.

In certain embodiments, an antibody is produced by expressing in a cell a polynucleotide encoding the VH and VL of an antibody described herein under suitable conditions so that the polynucleotides are expressed and the antibody is produced. In another embodiment, an antibody is produced by expressing in a cell a polynucleotide encoding the heavy chain and light chain of an antibody described herein under suitable conditions so that the polynucleotides are expressed and the antibody is produced. In certain embodiments, an antibody is produced by expressing in a cell a first polynucleotide encoding the VH of an antibody described herein, and a second polynucleotide encoding the VL of an antibody described herein, under suitable conditions so that the polynucleotides are expressed and the antibody is produced. In certain embodiments, an antibody is produced by expressing in a cell a first polynucleotide encoding the heavy chain of an antibody described herein, and a second polynucleotide encoding the light chain of an antibody described herein, under suitable conditions so that the polynucleotides are expressed and the antibody is produced.

Methods for producing polyclonal antibodies are known in the art (see, for example, Chapter 11 in: Short Protocols in Molecular Biology, (2002) 5th Ed., Ausubel F M et al., eds., John Wiley and Sons, New York, which is herein incorporated by reference in its entirety).

Monoclonal antibodies can be prepared using a wide variety of techniques known in the art, including the use of hybridoma, recombinant, and phage display technologies, or a combination thereof. For example, monoclonal antibodies can be produced using hybridoma techniques, including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), each of which is herein incorporated by reference in its entirety. The term “monoclonal antibody” as used herein is not limited to antibodies produced through hybridoma technology. For example, monoclonal antibodies can be produced recombinantly from host cells exogenously expressing an antibody described herein or a fragment thereof, for example, light chain and/or heavy chain of such antibody.

In specific embodiments, a “monoclonal antibody,” as used herein, is an antibody produced by a single cell (e.g., hybridoma or host cell producing a recombinant antibody), wherein the antibody specifically binds to TDP-43 (e.g., human TDP-43) as determined, e.g., by ELISA or other antigen-binding or competitive binding assay known in the art or in the examples provided herein. In certain embodiments, a monoclonal antibody can be a chimeric antibody or a humanized antibody. In certain embodiments, a monoclonal antibody is a monovalent antibody or multivalent (e.g., bivalent) antibody. In certain embodiments, a monoclonal antibody is a monospecific or multispecific antibody (e.g., bispecific antibody). Monoclonal antibodies described herein can, for example, be made by the hybridoma method as described in Kohler G & Milstein C (1975) Nature 256:495, which is herein incorporated by reference in its entirety, or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel F M et al., supra).

As used herein, an antibody binds to an antigen multivalently (e.g., bivalently) when the antibody comprises at least two (e.g., two or more) monovalent binding regions, each monovalent binding region capable of binding to an epitope on the antigen. Each monovalent binding region can bind to the same or different epitopes on the antigen.

Methods for producing and screening for specific antibodies using hybridoma technology are routine and well known in the art. For example, in the hybridoma method, a mouse or other appropriate host animal, such as a sheep, goat, rabbit, rat, hamster, or macaque monkey, is immunized to elicit lymphocytes that produce or are capable of producing antibodies that will specifically bind to the protein (e.g., TDP-43 (e.g., human TDP-43)) used for immunization. Alternatively, lymphocytes may be immunized in vitro. Lymphocytes then are fused with myeloma cells using a suitable fusing agent, such as polyethylene glycol, to form a hybridoma cell (Goding J W (Ed), Monoclonal Antibodies: Principles and Practice, pp. 59-103 (Academic Press, 1986), herein incorporated by reference in its entirety). Additionally, a RIMMS (repetitive immunization multiple sites) technique can be used to immunize an animal (Kilpatrick K E et al., (1997) Hybridoma 16:381-9, herein incorporated by reference in its entirety).

In certain embodiments, mice (or other animals, such as rats, monkeys, donkeys, pigs, sheep, hamster, or dogs) can be immunized with an antigen (e.g., TDP-43 (e.g., human TDP-43)) and once an immune response is detected, e.g., antibodies specific for the antigen are detected in the mouse serum, the mouse spleen is harvested and splenocytes isolated. The splenocytes are then fused by well-known techniques to any suitable myeloma cells, for example, cells from cell line SP20 available from the American Type Culture Collection (ATCC®) (Manassas, VA), to form hybridomas. Hybridomas are selected and cloned by limited dilution. In certain embodiments, lymph nodes of the immunized mice are harvested and fused with NS0 myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as the NS0 cell line or those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor D (1984) J Immunol 133:3001-5; Brodeur et al., Monoclonal Antibody Production Techniques and Applications, pp. 51-63 (Marcel Dekker, Inc., New York, 1987), each of which is herein incorporated by reference in its entirety).

Culture medium in which hybridoma cells are growing is assayed for production of monoclonal antibodies directed against TDP-43 (e.g., human TDP-43). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding J W (Ed), Monoclonal Antibodies: Principles and Practice, supra). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies described herein include, e.g., antibody fragments which recognize TDP-43 (e.g., human TDP-43), and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies described herein can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli, and the E. coli is infected with helper phage. Phages used in these methods are typically filamentous phage, including fd and M13, and the VH and VL domains are usually recombinantly fused to either the phage gene III or gene VIII. Phage expressing an antigen-binding region that binds to a particular antigen can be selected or identified with antigen, e.g., using labeled antigen or antigen bound or captured to a solid surface or bead. Examples of phage display methods that can be used to make the antibodies described herein include those disclosed in Brinkman U et al., (1995) J Immunol Methods 182:41-50; Ames R S et al., (1995) J Immunol Methods 184:177-186; Kettleborough C A et al., (1994) Eur J Immunol 24:952-958; Persic L et al., (1997) Gene 187:9-18; Burton D R & Barbas C F (1994) Advan Immunol 57:191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108, all of which are herein incorporated by reference in their entireties.

As described in the above references, after phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produce antibody fragments such as Fab, Fab′ and F(ab′)₂ fragments can also be employed using methods known in the art such as those disclosed in PCT publication No. WO 92/22324; Mullinax R L et al., (1992) BioTechniques 12 (6): 864-9; Sawai H et al., (1995) Am J Reprod Immunol 34:26-34; and Better M et al., (1988) Science 240:1041-1043, all of which are herein incorporated by reference in their entireties.

In certain embodiments, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison S L (1985) Science 229:1202-7; Oi V T & Morrison S L (1986) BioTechniques 4:214-221; Gillies S D et al., (1989) J Immunol Methods 125:191-202; and U.S. Pat. Nos. 5,807,715, 4,816,567, 4,816,397, and 6,331,415, all of which are herein incorporated by reference in their entireties.

A humanized antibody is capable of binding to a predetermined antigen and which comprises a framework region having substantially the amino acid sequence of a human immunoglobulin and CDRs having substantially the amino acid sequence of a non-human immunoglobulin (e.g., a murine immunoglobulin). In certain embodiments, a humanized antibody also comprises at least a portion of an immunoglobulin constant region (Fc), typically that of a human immunoglobulin. The antibody also can include the CH1, hinge, CH2, CH3, and CH4 regions of the heavy chain. A humanized antibody can be selected from any class of immunoglobulins, including IgM, IgG, IgD, IgA, and IgE, and any isotype, including IgG₁, IgG₂, IgG₃, and IgG₄. Humanized antibodies can be produced using a variety of techniques known in the art, including but not limited to, CDR-grafting (European Patent No. EP 239400; International Publication No. WO 91/09967; and U.S. Pat. Nos. 5,225,539, 5,530,101, and 5,585,089), veneering or resurfacing (European Patent Nos. EP 592106 and EP 519596; Padlan E A (1991) Mol Immunol 28 (4/5): 489-498; Studnicka G M et al., (1994) Prot Engineering 7 (6): 805-814; and Roguska M A et al., (1994) PNAS 91:969-973), chain shuffling (U.S. Pat. No. 5,565,332), and techniques disclosed in, e.g., U.S. Pat. Nos. 6,407,213, 5,766,886, International Publication No. WO 93/17105; Tan P et al., (2002) J Immunol 169:1119-25; Caldas C et al., (2000) Protein Eng. 13 (5): 353-60; Morea V et al., (2000) Methods 20 (3): 267-79; Baca M et al., (1997) J Biol Chem 272 (16): 10678-84; Roguska M A et al., (1996) Protein Eng 9 (10): 895 904; Couto J R et al., (1995) Cancer Res. 55 (23 Supp): 5973s-5977s; Couto J R et al., (1995) Cancer Res 55 (8): 1717-22; Sandhu J S (1994) Gene 150 (2): 409-10; and Pedersen J T et al., (1994) J Mol Biol 235 (3): 959-73, all of which are herein incorporated by reference in their entireties. See also, U.S. Application Publication No. US 2005/0042664 A1 (Feb. 24, 2005), which is herein incorporated by reference in its entirety.

Methods for making multispecific antibodies (e.g., bispecific antibodies) have been described, see, for example, U.S. Pat. Nos. 7,951,917; 7,183,076; 8,227,577; 5,837,242; 5,989,830; 5,869,620; 6,132,992; and 8,586,713, all of which are herein incorporated by reference in their entireties.

Bispecific, bivalent antibodies, and methods of making them, are described, for instance in U.S. Pat. Nos. 5,731,168, 5,807,706, 5,821,333, and U.S. Appl. Publ. Nos. 2003/020734 and 2002/0155537; each of which is herein incorporated by reference in its entirety. Bispecific tetravalent antibodies, and methods of making them are described, for instance, in Int. Appl. Publ. Nos. WO 02/096948 and WO 00/44788, the disclosures of both of which are herein incorporated by reference in its entirety. See generally, Int. Appl. Publ. Nos. WO 93/17715, WO 92/08802, WO 91/00360, and WO 92/05793; Tutt et al., J. Immunol. 147:60-69 (1991); U.S. Pat. Nos. 4,474,893; 4,714,681; 4,925,648; 5,573,920; and 5,601,819; and Kostelny et al., J. Immunol. 148:1547-1553 (1992); each of which is herein incorporated by reference in its entirety.

A bispecific antibody as described herein can be generated according to the DuoBody technology platform (Genmab A/S) as described, e.g., in International Publication Nos. WO 2011/131746, WO 2011/147986, WO 2008/119353, and WO 2013/060867, and in Labrijn A F et al., (2013) PNAS 110 (13): 5145-5150. The DuoBody technology can be used to combine one half of a first monospecific antibody, or first antigen-binding region, containing two heavy and two light chains with one half of a second monospecific antibody, or second antigen-binding region, containing two heavy and two light chains. The resultant heterodimer contains one heavy chain and one light chain from the first antibody, or first antigen-binding region, paired with one heavy chain and one light chain from the second antibody, or second antigen-binding region. When both of the monospecific antibodies, or antigen-binding regions, recognize different epitopes on different antigens, the resultant heterodimer is a bispecific antibody.

The DuoBody technology requires that each of the monospecific antibodies, or antigen-binding regions includes a heavy chain constant region with a single point mutation in the CH3 domain. The point mutations allow for a stronger interaction between the CH3 domains in the resultant bispecific antibody than between the CH3 domains in either of the monospecific antibodies, or antigen-binding regions. The single point mutation in each monospecific antibody, or antigen-binding region, is at residue 366, 368, 370, 399, 405, 407, or 409, numbered according to the EU numbering system, in the CH3 domain of the heavy chain constant region, as described, e.g., in International Publication No. WO 2011/131746. Moreover, the single point mutation is located at a different residue in one monospecific antibody, or antigen-binding region, as compared to the other monospecific antibody, or antigen-binding region. For example, one monospecific antibody, or antigen-binding region, can comprise the mutation F405L (i.e., a mutation from phenylalanine to leucine at residue 405), while the other monospecific antibody, or antigen-binding region, can comprise the mutation K409R (i.e., a mutation from lysine to arginine at residue 409), numbered according to the EU numbering system. The heavy chain constant regions of the monospecific antibodies, or antigen-binding regions, can be an IgG₁, IgG₂, IgG₃, or IgG₄ isotype (e.g., a human IgG₁ isotype), and a bispecific antibody produced by the DuoBody technology can retain Fc-mediated effector functions.

Another method for generating bispecific antibodies has been termed the “knobs-into-holes” strategy (see, e.g., Intl. Publ. WO2006/028936). The mispairing of Ig heavy chains is reduced in this technology by mutating selected amino acids forming the interface of the CH3 domains in IgG. At positions within the CH3 domain at which the two heavy chains interact directly, an amino acid with a small side chain (hole) is introduced into the sequence of one heavy chain and an amino acid with a large side chain (knob) into the counterpart interacting residue location on the other heavy chain. In some embodiments, compositions of the disclosure have immunoglobulin chains in which the CH3 domains have been modified by mutating selected amino acids that interact at the interface between two polypeptides so as to preferentially form a bispecific antibody. The bispecific antibodies can be composed of immunoglobulin chains of the same subclass (e.g., IgG₁ or IgG₃) or different subclasses (e.g., IgG₁ and IgG₃, or IgG₃ and IgG₄).

Bispecific antibodies can, in some instances contain, IgG₄ and IgG₁, IgG₄ and IgG₂, IgG₄ and IgG₃, or IgG₁ and IgG₃ chain heterodimers. Such heterodimeric heavy chain antibodies can routinely be engineered by, for example, modifying selected amino acids forming the interface of the CH3 domains in human IgG₄ and the IgG₁ or IgG₃, so as to favor heterodimeric heavy chain formation.

In certain embodiments, an antibody described herein, which binds to the same epitope of TDP-43 (e.g., human TDP-43) as an anti-TDP-43 (e.g., human TDP-43) antibody described herein, is a human antibody. In certain embodiments, an antibody described herein, which competitively blocks (e.g., in a dose-dependent manner) any one of the antibodies described herein, from binding to TDP-43 (e.g., human TDP-43), is a human antibody. Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., TDP-43 (e.g., human TDP-43)). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM, and IgE antibodies. For an overview of this technology for producing human antibodies, see Lonberg N & Huszar D (1995) Int Rev Immunol 13:65-93, herein incorporated by reference in its entirety. For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, all of which are herein incorporated by reference in their entireties. Examples of mice capable of producing human antibodies include the XenoMouse™ (Abgenix, Inc.; U.S. Pat. Nos. 6,075,181 and 6,150,184), the HuAb-Mouse™ (Medarex, Inc./Gen Pharm; U.S. Pat. Nos. 5,545,806 and 5,569,825), the Trans Chromo Mouse™ (Kirin) and the KM Mouse™ (Medarex/Kirin), all of which are herein incorporated by reference in their entireties.

Human antibodies that specifically bind to TDP-43 (e.g., human TDP-43) can be made by a variety of methods known in the art, including the phage display methods described above using antibody libraries derived from human immunoglobulin sequences. See also, U.S. Pat. Nos. 4,444,887, 4,716,111, and 5,885,793; and International Publication Nos. WO 98/46645, WO 98/50433, WO 98/24893, WO 98/16654, WO 96/34096, WO 96/33735, and WO 91/10741, all of which are herein incorporated by reference in their entireties.

In certain embodiments, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that specifically bind to a target antigen (e.g., TDP-43 (e.g., human TDP-43)). Such methods are known and are described in the art, see, e.g., Shinmoto H et al., (2004) Cytotechnology 46:19-23; Naganawa Y et al., (2005) Human Antibodies 14:27-31, each of which is herein incorporated by reference in its entirety.

### Pharmaceutical Compositions

Provided herein are compositions comprising an anti-TDP-43 antibody disclosed herein having the desired degree of purity in a physiologically acceptable carrier, excipient, or stabilizer (see, e.g., Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates, including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In a specific embodiment, pharmaceutical compositions comprise an anti-TDP-43 antibody disclosed herein, and optionally one or more additional prophylactic or therapeutic agents, in a pharmaceutically acceptable carrier. In a specific embodiment, pharmaceutical compositions comprise an anti-TDP-43 antibody disclosed herein, and optionally one or more additional prophylactic or therapeutic agents, in a pharmaceutically acceptable carrier. In certain embodiments, the antibody is the only active ingredient included in the pharmaceutical composition. Pharmaceutical compositions described herein can be useful in reducing TDP-43 (e.g., human TDP-43) protein aggregates, inhibiting an activity of TDP-43 (e.g., human TDP-43), and treating a disease or disorder associated with TDP-43. In certain embodiments, the present disclosure relates to a pharmaceutical composition of the present disclosure comprising an anti-TDP-43 antibody of the present disclosure for use as a medicament. In another embodiment, the present disclosure relates to a pharmaceutical composition of the present disclosure for use in a method for the treatment of a disease or disorder associated with TDP-43 or TDP-43 proteinopathy.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering, or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil, and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride, and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol, and propylene glycol for water miscible vehicles; and sodium hydroxide, hydrochloric acid, citric acid, or lactic acid for pH adjustment.

A pharmaceutical composition can be formulated for any route of administration to a subject. Specific examples of routes of administration include intranasal, oral, pulmonary, transdermal, intradermal, and parenteral. In some embodiments, the antibody, polynucleotide, rAAV, host cell, or composition is delivered to the central nervous system (CNS). In some embodiments, the antibody, polynucleotide, rAAV, host cell, or composition is delivered intravenously, intrathecally, intracisternally, intraparenchymally, intravitreally, or subretinally.

Parenteral administration, characterized by either subcutaneous, intramuscular, or intravenous injection, is also contemplated herein. Injectables can be prepared in conventional forms, either as liquid solutions or suspensions, solid forms suitable for solution or suspension in liquid prior to injection, or as emulsions. The injectables, solutions, and emulsions also contain one or more excipients. Suitable excipients are, for example, water, saline, dextrose, glycerol, or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate, and cyclodextrins.

Preparations for parenteral administration of antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol, and mixtures thereof.

Topical mixtures comprising an antibody are prepared as described for the local and systemic administration. The resulting mixture can be a solution, suspension, emulsions, or the like and can be formulated as creams, gels, ointments, emulsions, solutions, elixirs, lotions, suspensions, tinctures, pastes, foams, aerosols, irrigations, sprays, suppositories, bandages, dermal patches, or any other formulations suitable for topical administration.

An anti-TDP-43 antibody disclosed herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma and are herein incorporated by reference in their entireties). These formulations for administration to the respiratory tract can be in the form of an aerosol or solution for a nebulizer, or as a microfine powder for insufflations, alone or in combination with an inert carrier such as lactose. In such a case, the particles of the formulation will, in certain embodiments, have diameters of less than 50 microns, In certain embodiments less than 10 microns.

An anti-TDP-43 antibody disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, all of which are herein incorporated by reference in their entireties.

In certain embodiments, a pharmaceutical composition comprising antibody described herein is a lyophilized powder, which can be reconstituted for administration as solutions, emulsions, and other mixtures. It may also be reconstituted and formulated as solids or gels. The lyophilized powder is prepared by dissolving antibody described herein, or a pharmaceutically acceptable derivative thereof, in a suitable solvent. In certain embodiments, the lyophilized powder is sterile. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose, or other suitable agent. The solvent may also contain a buffer, such as citrate, sodium or potassium phosphate or other such buffer known to those of skill in the art at, In certain embodiments, about neutral pH. Subsequent sterile filtration of the solution followed by lyophilization under standard conditions known to those of skill in the art provides the desired formulation. In certain embodiments, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

The anti-TDP-43 antibodies disclosed herein and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, all of which are herein incorporated by reference in their entireties. In a specific embodiment, an antibody described herein is targeted to the central nervous system (CNS). In a specific embodiment, an antibody described herein is targeted to a motor neuron in the CNS.

The compositions to be used for in vivo administration can be sterile. This is readily accomplished by filtration through, e.g., sterile filtration membranes.

### Methods of Use and Uses

In certain embodiments, the instant disclosure provides an antibody that specifically binds to TDP-43 (e.g., human TDP-43), wherein the antibody binds to aggregated TDP-43. In some embodiments, the antibody binds to phosphorylated TDP-43. In some embodiments, the antibody binds to non-phosphorylated TDP-43.

In certain embodiments, the instant disclosure provides an antibody that specifically binds to TDP-43 (e.g., human TDP-43), wherein the antibody reduces levels of aggregated and/or phosphorylated TDP-43 in a cell; restores full-length Stathmin-2 (STMN2) expression; reduces STMN2 cryptic exon (CE) mRNA expression; increases STMN2 protein in neurites as measured with neurite outgrowth, in motor neurons exposed to a cellular stressor; reduces cellular toxicity in response to a cellular stressor; and/or restores motor neuron function as assessed by MEA system.

In certain embodiments, the instant disclosure provides an antibody described herein that specifically binds to TDP-43 (e.g., human TDP-43), a polynucleotide encoding the antibody, or an rAAV comprising the polynucleotide, for use in the treatment of a disease or disorder associated with TDP-43 or TDP-43 proteinopathy.

In some embodiments, the disease or disorder associated with TDP-43 or TDP-43 proteinopathy is selected from the group consisting of amyotrophic lateral sclerosis (ALS, including sporadic and familial forms), frontotemporal dementia (FTD, including sporadic and familial forms), Alzheimer's disease (AD, including sporadic and familial forms), Inclusion body myositis (IBM), Oculopharyngeal muscular dystrophy (OPMD), Down syndrome, Familial British dementia, Polyglutamine diseases (including Huntington's disease and spinocerebellar ataxia type 3 (SCA3; also known as Machado Joseph Disease)), Dementia with Lewy Bodies (DLB), or Parkinson's disease (PD). In a specific embodiment, the disease or disorder is amyotrophic lateral sclerosis (ALS, including sporadic and familial forms).

In some embodiments, treatment of the disease or disorder associated with TDP-43 or TDP-43 proteinopathy comprises delivery of the antibody, polynucleotide, or rAAV to the CNS. In some embodiments, the antibody, polynucleotide, or rAAV is administered intravenously, intrathecally, intracisternally, intraparenchymally, intravitreally, or subretinally. In some embodiments, treatment of the disease or disorder associated with TDP-43 or TDP-43 proteinopathy comprises expression of the antibody in motor neurons in the CNS. In some embodiments, treatment of the disease or disorder associated with TDP-43 or TDP-43 proteinopathy comprises a reduction of human TDP-43 protein aggregates in the CNS.

In an aspect, provided herein is a method of inhibiting an activity of TDP-43 in a subject, the method comprising administering to the subject a therapeutically effective amount of an antibody or polypeptide that specifically binds to TDP-43 (e.g., human TDP-43); a polynucleotide encoding an antibody or polypeptide that specifically binds to TDP-43 (e.g., human TDP-43); an rAAV comprising a polynucleotide encoding an antibody or polypeptide that specifically binds to TDP-43 (e.g., human TDP-43); a host cell comprising a polynucleotide encoding an antibody or polypeptide that specifically binds to TDP-43 (e.g., human TDP-43); or a composition comprising an antibody or a polynucleotide encoding an antibody or polypeptide that specifically binds to TDP-43 (e.g., human TDP-43). In some embodiments, the activity of TDP-43 is aggregate-induced neurotoxicity.

In an aspect, provided herein is a method of preventing or reducing TDP-43 protein aggregates in a subject, the method comprising administering to the subject a therapeutically effective amount of an antibody or polypeptide that specifically binds to TDP-43 (e.g., human TDP-43); a polynucleotide encoding an antibody or polypeptide that specifically binds to TDP-43 (e.g., human TDP-43); an rAAV comprising a polynucleotide encoding an antibody or polypeptide that specifically binds to TDP-43 (e.g., human TDP-43); a host cell comprising a polynucleotide encoding an antibody or polypeptide that specifically binds to TDP-43 (e.g., human TDP-43); or a composition comprising an antibody or a polynucleotide encoding an antibody or polypeptide that specifically binds to TDP-43 (e.g., human TDP-43).

In an aspect, provided herein is a method of treating a disease or disorder associated with TDP-43 in a subject, the method comprising administering to the subject a therapeutically effective amount of an antibody or polypeptide that specifically binds to TDP-43 (e.g., human TDP-43); a polynucleotide encoding an antibody or polypeptide that specifically binds to TDP-43 (e.g., human TDP-43); an rAAV comprising a polynucleotide encoding an antibody or polypeptide that specifically binds to TDP-43 (e.g., human TDP-43); a host cell comprising a polynucleotide encoding an antibody or polypeptide that specifically binds to TDP-43 (e.g., human TDP-43); or a composition comprising an antibody or a polynucleotide encoding an antibody or polypeptide that specifically binds to TDP-43 (e.g., human TDP-43). In certain embodiments, the disease or disorder is a TDP-43 proteinopathy.

In an embodiment, the subject has Amyotrophic lateral sclerosis (ALS), Frontotemporal dementia (FTD), Alzheimer's disease (AD), Inclusion body myositis (IBM), Oculopharyngeal muscular dystrophy (OPMD), Down syndrome, Familial British dementia, Polyglutamine diseases, Huntington's disease, spinocerebellar ataxia type 3 (SCA3), Dementia with Lewy Bodies (DLB), or Parkinson's disease (PD). In an embodiment, the subject has amyotrophic lateral sclerosis (ALS, including sporadic and familial forms).

In an embodiment, the method prevents or reduces TDP-43 aggregates. In an embodiment, the method results in about a 1, 5, 10, 20, 30, 40, 50, 60, 70, 80, 90, or 100% reduction in TDP-43 aggregates in the subject.

In an embodiment, the method neutralizes TDP-43 mediated neurotoxicity. In an embodiment, the method increases electrical firing of a neuron. In an embodiment, the method increases neurite outgrowth on a neuron.

In an embodiment, the vector targets a cortical neuron, a spinal neuron, and/or an astrocyte.

In an embodiment, the subject is a human subject.

In an embodiment, the method further comprises administering an additional therapeutic agent.

In an aspect, provided herein is an antibody that specifically binds to TDP-43 or a polynucleotide encoding an antibody that specifically binds to TDP-43 for use in inhibiting an activity of TDP-43 in a subject in need thereof, wherein the treatment is performed according to any one of the methods disclosed herein. In certain embodiments, the activity is TDP-43 aggregate-induced neurotoxicity.

In an aspect, provided herein is an antibody that specifically binds to TDP-43 or a polynucleotide encoding an antibody that specifically binds to TDP-43 for use in preventing or reducing TDP-43 aggregates in a subject in need thereof, wherein the treatment is performed according to any one of the methods disclosed herein.

In an aspect, provided herein is an antibody that specifically binds to TDP-43 or a polynucleotide encoding an antibody that specifically binds to TDP-43 for use in the manufacture of a medicament for inhibiting TDP-43 aggregate-induced neurotoxicity in a subject in need thereof, wherein the treatment is performed according to any one of the methods disclosed herein.

In an aspect, provided herein is an antibody that specifically binds to TDP-43 or a polynucleotide encoding an antibody that specifically binds to TDP-43 for use in the manufacture of a medicament for treating a disease or disorder associated with TDP-43 in a subject in need thereof, wherein the treatment is performed according to any one of the methods disclosed herein.

In an aspect, provided herein is an antibody that specifically binds to TDP-43 or a polynucleotide encoding an antibody that specifically binds to TDP-43 for use in the manufacture of a medicament for preventing or reducing TDP-43 aggregates in a subject in need thereof, wherein the treatment is performed according to any one of the methods disclosed herein.

In an aspect, provided herein is a use of an antibody that specifically binds to TDP-43 or a polynucleotide encoding an antibody that specifically binds to TDP-43 for inhibiting TDP-43 aggregate-induced neurotoxicity in a subject in need thereof, wherein the treatment is performed according to any one of the methods disclosed herein.

### Kits

Also provided are kits comprising one or more antibodies described herein, or pharmaceutical compositions or conjugates thereof. In a specific embodiment, provided herein is a pharmaceutical pack or kit comprising one or more containers filled with one or more of the ingredients of the pharmaceutical compositions described herein, such as one or more antibodies provided herein. In certain embodiments, the kits contain a pharmaceutical composition described herein and any prophylactic or therapeutic agent, such as those described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided, are kits that can be used in the above methods. In certain embodiments, a kit comprises an antibody described herein, preferably purified antibody, in one or more containers. In a specific embodiment, kits described herein contain a substantially isolated TDP-43 (e.g., human TDP-43) antigen as a control. In another specific embodiment, the kits described herein further comprise a control antibody which does not react with TDP-43 (e.g., human TDP-43) antigen. In another specific embodiment, kits described herein contain one or more elements for detecting the binding of an antibody to an TDP-43 (e.g., human TDP-43) antigen (e.g., the antibody can be conjugated to a detectable substrate such as a fluorescent compound, an enzymatic substrate, a radioactive compound, or a luminescent compound, or a second antibody which recognizes the first antibody can be conjugated to a detectable substrate). In specific embodiments, a kit provided herein can include a recombinantly produced or chemically synthesized TDP-43 (e.g., human TDP-43) antigen. The TDP-43 (e.g., human TDP-43) antigen provided in the kit can also be attached to a solid support. In a more specific embodiment, the detecting means of the above-described kit includes a solid support to which an TDP-43 (e.g., human TDP-43) antigen is attached. Such a kit can also include a non-attached reporter-labeled anti-human antibody or anti-mouse/rat antibody. In this embodiment, binding of the antibody to the TDP-43 (e.g., human TDP-43) antigen can be detected by binding of the said reporter-labeled antibody. In certain embodiments, the present disclosure relates to the use of a kit of the present disclosure for in vitro assaying and/or detecting TDP-43 (e.g., human TDP-43) antigen in a biological sample.

### EXAMPLES

### Example 1: Analysis of TDP-43 scFvs

Cytosolic aggregates, which act as inclusion bodies in patients with ALS and frontotemporal lobar degeneration (FTLD) are mimicked in in vitro cellular systems using a mutant form of TDP-43. Targeting TDP-43 can reduce pathological, aggregated TDP-43. TDP43-ΔNLS1-2KQ is an aggregate-prone mutant lacking a nuclear localization sequence, with acetylation-mimic substitutions K145Q and K192Q. Expression of TDP43-ΔNLS1-2KQ in cells leads to robust TDP-43 inclusions resembling ALS pathology in the cytosol. TDP43-ΔNLS1-2KQ was labeled with EGFP and used as a model for the following experiments.

A panel of humanized TDP-43 scFvs were generated and analyzed for their capacity to reduce TDP-43 aggregates in U2OS human osteosarcoma cells. U2OS cells were seeded at 8000 cells per well in a 96-well polymer bottom plate (ibidi) cultured in DMEM supplemented with 5% FCS. The cells were cultured in an environment at 37° C. supplemented with 5% CO2. The following day, cells were co-transfected in triplicates with 10 ng of TDP43-ΔNLS1-2KQ-EGFP and either 50 ng of anti-TDP-43 scFv (AAV5.2-scFv, i.e., VecTab clones AA-BI, see Table 1-4) or a non-TDP-43 scFv using Lipofectamine 3000. Two days after transfection, the cells were fixed using 3.7% formaldehyde in TBS and permeabilized with 0.1% Triton-X-100 in TBS, followed by blocking with 5% BSA in TBS. Finally, the nuclei were stained using Hoechst. The GFP signal was used to quantify the TDP-43 aggregates. The cells were imaged using ImageXpress Micro-Confocal at 25 different locations in each well, and TDP-43 aggregates were quantified using MetaXpress 6. The level of TDP-43 aggregates per cell was normalized to control cells transfected with non-TDP-43 binding scFv.

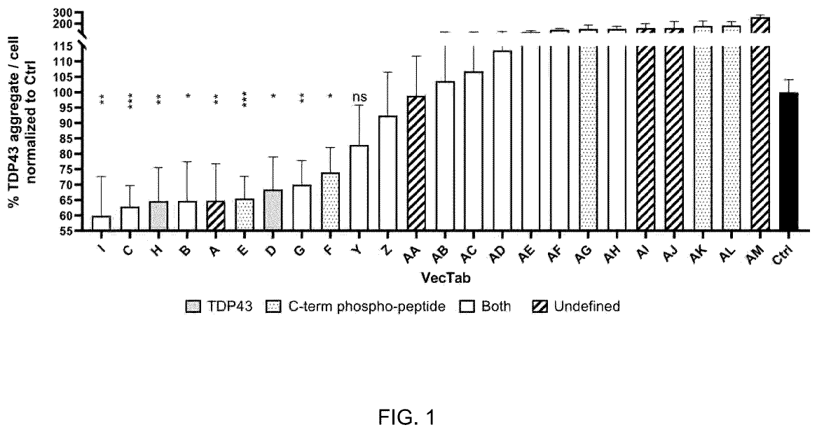

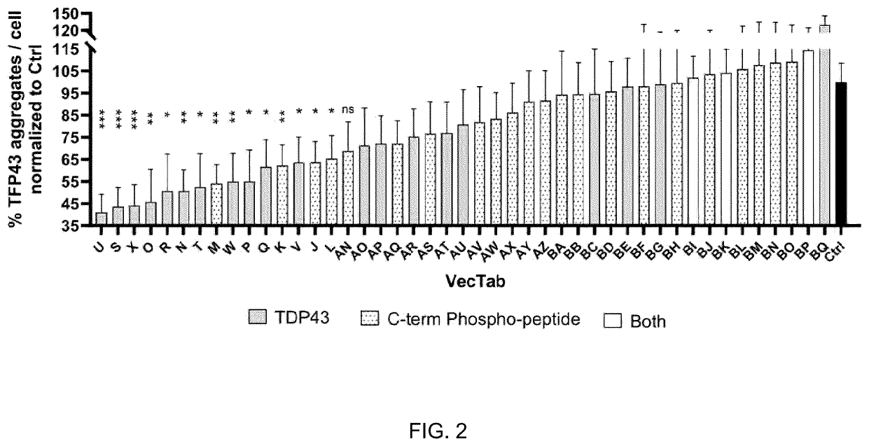

The results shown in FIG. 1 and FIG. 2 show that several of the anti-TDP-43 scFvs significantly decreased levels of TDP-43 aggregates per cell, compared to cells transfected with a non-TDP-43 scFv, which had no effect on TDP-43 aggregates. VecTabs shown in FIG. 1 were predicted to bind within or near amino acid sequence 397-411 of human TDP43. VecTabs shown in FIG. 2 were predicted to bind within or near amino acid sequence 387-411 of human TDP43. VecTab B, VecTab G, VecTab I, VecTab S, and VecTab U were chosen for additional testing.

### Example 2: Restoration of Nuclear TDP-43

TDP-43-related neurodegeneration involves alterations in the normal physiological state of TDP-43, including nuclear depletion and mislocalization into the cytoplasm. The simultaneous decrease and impairment of nuclear TDP-43 in cells plays a crucial role in toxic cytoplasmic aggregation and neurodegenerative pathology. Targeting TDP-43 aggregates may restore levels of nuclear TDP-43.

iCell motor neurons bearing the ALS-associated M337V mutation in TDP-43 (M337V, iCell, Catalog #: R1145) were thawed, seeded, and maintained in 384-well plate format at a cell density of 8000 cells per well. The motor neurons were transduced seven days post-seeding with VecTab B, VecTab G, VecTab I, VecTab S, or VecTab U at MOI 10\hat{ }6 for two weeks, and control cells were untreated (NT). To induce nuclear depletion, M337V motor neurons were exposed to MG132 stressor at a concentration of 0.1 μM for seven days at one week post-transduction.

M337V motor neurons were subsequently fixed with 4% formaldehyde and cells were permeabilized with 0.5% Triton-X-100 in 1xTBS for 15 min. Excess Triton-X-100 was removed by washing cells 3× 5 min with 1xTBS. Nonspecific binding was blocked with 3% BSA in 1xTBS for 45 min. Anti-TDP-43 primary antibody (10782-2-AP, Proteintech) was diluted in 3% BSA in 1xTBS, and cells were incubated overnight at 4° C. The next day, the primary antibody solution was removed, and cells were washed 3× 5 min with 1xTBS. Secondary antibody solution goat anti-rabbit 488 (A32731, Invitrogen) was prepared in 3% BSA in 1xTBS and cells were incubated for 45 min at RT. To remove background staining, cells were washed 2× 5 min with 1xTBS, and nuclei were stained with Hoechst for 10 min. 2× 5 min washes with 1xTBS were performed to remove Hoechst and 1xPBS was added to cells before the wells were covered with a seal to prevent evaporation. To visualize fluorescent staining, cells were imaged on the ImageXpress Micro Confocal High-Content Imaging System. All images were acquired with the same exposure time between conditions. Nuclear TDP-43 average intensity was measured using macros from ImageXpress® Micro Confocal High-Content Imaging System. At least 1500 cells per well were measured. Each group contained 7 replicates.

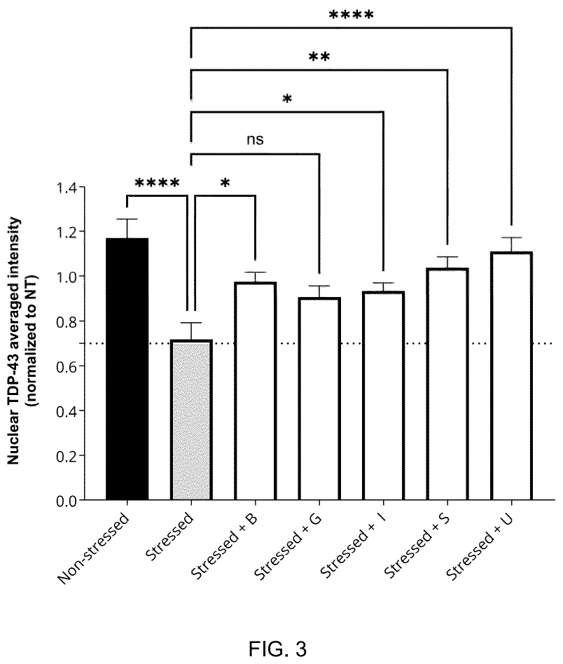

The results in FIG. 3 show depletion of nuclear TDP-43 upon treatment of M337V motor neurons with the MG132 stressor, with restoration of nuclear TDP-43 when the cells were treated with VecTab B, VecTab G, VecTab I, VecTab S, or VecTab U.

### Example 3: Restoration of Stathmin-2 (STMN2) mRNA Splicing

Stathmin-2 (STMN2) is a nervous system-specific phosphoprotein that regulates microtubule stability and axon outgrowth. STMN2 protein has been shown to be reduced in the spinal cord of most ALS patients, and STMN2 mis-splicing events are also associated with ALS. TDP-43 mediates proper STMN2 mRNA splicing, and TDP-43 loss of function results in dramatic reduction of STMN2 mRNA functional protein. Specifically, TDP-43 pathology leads to splicing defects and the production of STMN2 cryptic exon (CE)-transcripts, which result in the loss of functional STMN2 in the cell (Mehta et al. 2023).

M337V motor neurons were thawed, seeded, and maintained in 384-well plate format at a cell density of 8000 cells per well for image-based analysis, and 12-well plate format at a cell density of 350,000 cells per well for RNA analysis. The motor neurons were transduced seven days post-seeding with VecTab B, VecTab G, VecTab I, VecTab S, or VecTab U at MOI 10\hat{ }6 and control cells were untreated (NT). To induce STMN2 mis-spicing events, M337V motor neurons were exposed to MG132 stressor at a concentration of 0.5 μM for 48 hours and/or 0.1 μM for seven days at seven and/or 14 days post-transduction.

For RNA isolation, cDNA synthesis, and RT-qPCR, M337V motor neurons were harvested by incubating and scraping in Qiazol, and stored at −80° C. until RNA isolation. The miRNeasy Mini kit was used for isolation of RNA according to manufacturer manual (miRNeasy Kits, Qiagen Cat. No./ID: 217084). cDNA was synthesized with the Maxima First Strand cDNA Synthesis Kit (Thermo-Fisher Scientific) for RT-qPCR. STMN2 full-length and STMN2 cryptic mRNA was measured with TaqMan™ Real-Time PCR and normalized to housekeeping gene GAPDH.

For immunohistochemistry, M337V motor neurons were fixed with 4% formaldehyde and cells were permeabilized with 0.5% Triton-X-100 in 1xTBS for 15 min. Excess Triton-X-100 was removed by washing cells for 3× 5 min with 1xTBS. Nonspecific binding was blocked with 3% BSA in 1xTBS for 45 min. Anti-STMN2 primary antibody (67204-1-1g, Proteintech) was diluted in 3% BSA in 1xTBS, and cells were incubated overnight at 4° C. The next day, the primary antibody solution was removed, and cells were washed 3× 5 min with 1xTBS. Secondary antibody solution goat anti-Mouse568 (A11004, Invitrogen) was prepared in 3% BSA in 1xTBS and cells were incubated for 45 min at RT. To remove background staining, cells were washed 2× 5 min with 1xTBS and nuclei were stained with Hoechst for 10 min. 2× 5 min washes with 1xTBS were performed to remove Hoechst and 1xPBS was added to cells before the wells were covered with a seal to prevent evaporation. To visualize fluorescent staining, cells were imaged on the ImageXpress Micro Confocal High-Content Imaging System. All images were acquired with the same exposure time between conditions. STMN2 protein was measured with the neurite tracing analyses, using macros from ImageXpress® Micro Confocal High-Content Imaging System. At least 1500 cells per well were measured. Each group contained 7 replicates.

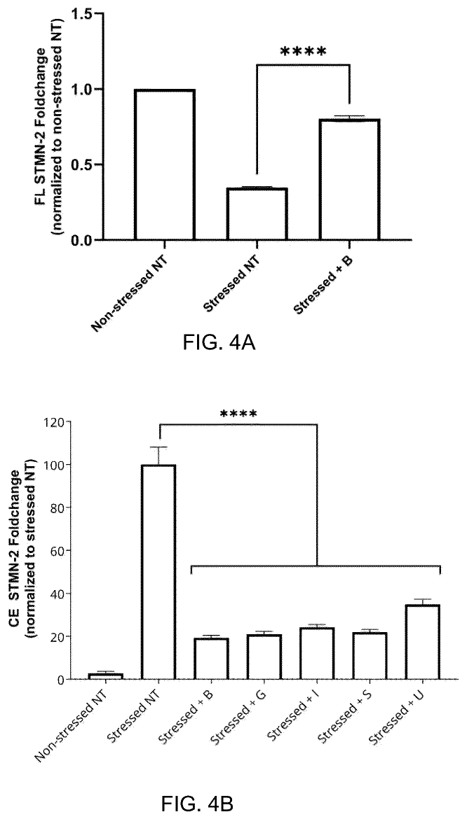

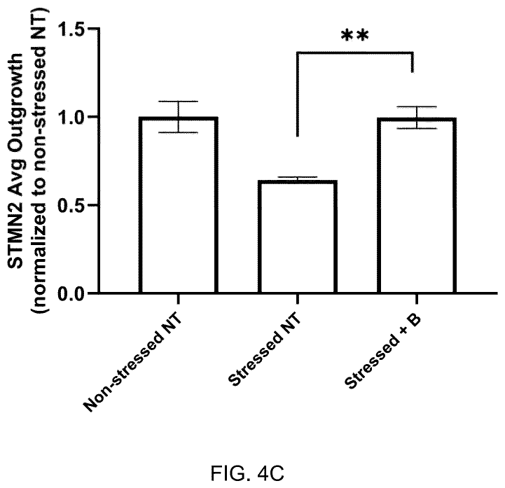

The results shown in FIG. 4A demonstrate depletion of STMN2 full-length mRNA upon treatment of M337V motor neurons with the MG132 stressor, which did not occur when the cells were pretreated with VecTab B. FIG. 4B shows that VecTab B, VecTab G, VecTab I, VecTab S, and VecTab U were able to reverse the increase in STMN2 CE-mRNA in M337V motor neurons with the MG132 stressor. FIG. 4C shows depletion of STMN2 protein in neurites of motor neurons, as measured by neurite outgrowth, upon treatment of M337V motor neurons with the MG132 stressor, and restoration of STMN2 protein in neurites when the stressed cells were treated with VecTab B.

### Example 4: Improvement of Cell Viability

A decrease in cell viability is observed in MG132 stressed M337V motor neurons.

To assess the effect of VecTabs on cell viability in stressed motor neurons, M337V motor neurons were plated in 384-well plates for immunocytochemistry (16,000 cells per well). Transduction with VecTab was performed on day 7 post-thaw. Half of the medium (25 μL) was removed from the well and 12.5 μL of either VecTab B, VecTab G, VecTab I, VecTab S, or VecTab U was added dropwise at MOI 10\hat{ }6. Twenty-four hours after transduction, 12.5 μL of fresh medium was added to the wells. Chronic MG132 (0.1 μM) treatment from days 14-21 post-thaw was performed. As a vehicle control, 0.1% DMSO from day 14-21 post-thaw was used. Plates were fixed on day 21 and quantification of toxicity was performed by nuclear cell count, labeling cells with Hoechst. Each group contained 7 replicates.

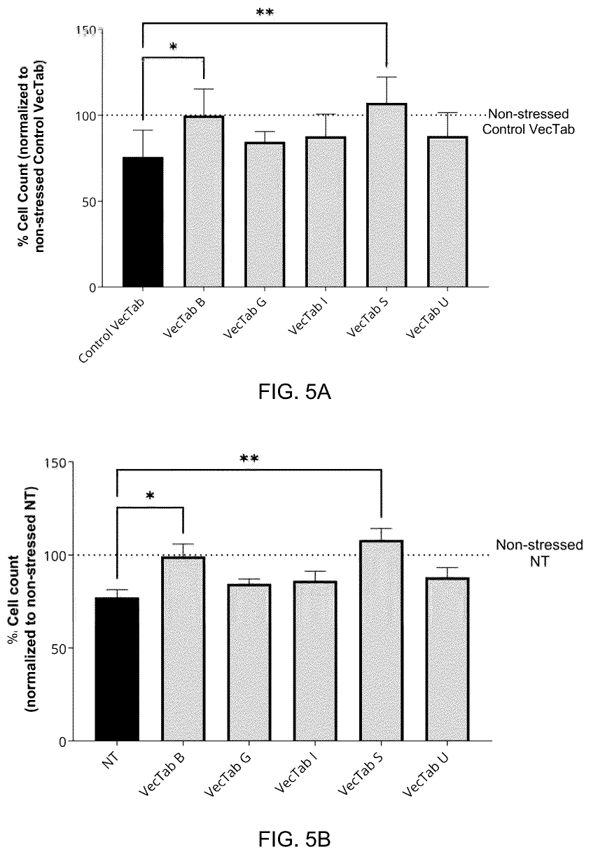

The results in FIGS. 5A-5B show a decrease in cell viability in M337V motor neurons treated with MG132 stressor and control VecTab or untreated (NT), and improved cell viability in stressed cells treated with VecTabs.

### Example 5: Improvement of Neuronal Function (Burst)

M337V motor neurons exhibit decreased neuronal function which can be measured with MEA system.

To assess whether VecTabs can improve neuronal function, wild type (WT; iCell, Catalog #: R1051) and M337V motor neurons were seeded at a density of 125,000 cells per well and co-cultured with astrocytes (iCell, Catalog #: R1240) at a cell density of 25,000 cells per well in Cytoview 48 well plates. The cells were transduced with either VecTab B, VecTab G, VecTab I, VecTab S, or VecTab U on day 7 post-thaw. To transduce the cells, half of the medium was removed from the well (150 μL) and 75 μL of VecTabs was added dropwise at MOI 10\hat{ }6. Twenty-four hours after transduction, 75 μL of fresh medium was added to the wells. Microelectrode array (MEA) activity was recorded twice per week for a maximum of 3 weeks. Numbers of burst frequency were determined at day 16 post-thaw, comparative statistical analysis was performed, and percentage rescue was calculated. Each group contained 6 replicates.

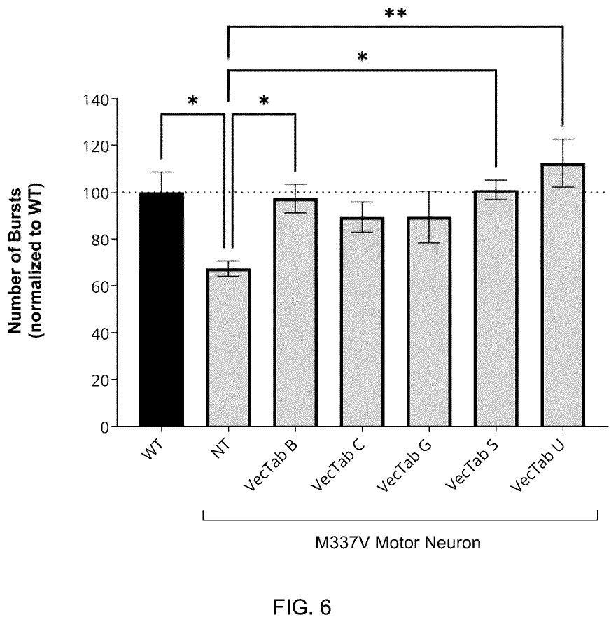

The results shown in FIG. 6 demonstrate a decrease in MEA activity in non-treated M337V motor neurons as compared to wild type cells, and improved MEA activity in motor neurons treated with VecTabs.

### Example 6: Binding to Non-Phosphorylated and Phosphorylated TDP-43

The ability of clones to bind phosphorylated TDP-43 and/or non-phosphorylated TDP-43 was assessed using N-terminally biotinylated TDP-43 peptides having the following sequences: NAGSGSGFNGGFGSSMDSKSSGWGM (SEQ ID NO: 218; non-phosphorylated); NAGSGSGFNGGFGSSMDSK {PSER} SGWGM (SEQ ID NO: 219; phosphorylated S409); NAGSGSGFNGGFGSSMDSKS {PSER} GWGM (SEQ ID NO: 220; phosphorylated S410); and NAGSGSGFNGGFGSSMDSK {PSER} {PSER} GWGM (SEQ ID NO: 221; phosphorylated S409/S410). Peptides were dissolved in 100% DMSO, aliquoted, and stored in −80° C. before use. Wells were incubated with 100 μL (5 μg/mL) of the peptides on streptavidin plates overnight at 4° C. The plates were then washed 3× in PBST wash buffer. Antibody clones B, C, G, I, O, S, U, and X, and non-binding controls (VRC07 for full-length IgG, and anti-methamphetamine clone 6H4 as control for the scFv experiments) were made in full-length human IgG₁ and scFv formats (IpA), and dilutions were prepared in blocking buffer (2% BSA). 100 μL/well of the dilutions were added to coated plates and the plates were incubated at RT for 1 hour. The plates were subsequently washed 3× with 250 μL/well wash buffer. 100 μL/well of primary antibody CEN63-C13 Rb-a-G4S (IpA) diluted in blocking buffer (2 μg/ml) was added and the plates were incubated for 1 hour at RT followed by 3× washing in PBST. For detection, 100 μL/well secondary antibodies (goat anti human IgG₁ HRP (Jackson ImmunoResearch) or goat anti-rabbit HRP for scFv (Promega)) diluted in blocking buffer (1:5000) was added to the wells and the plates were incubated for 1 hour at RT. The plates were subsequently washed 3× and 100 μL/well of TMB ready solution was added to the wells, followed by 3-4 minutes incubation in the dark. The reaction was stopped with 1M H₂SO₄ added to the wells (100 μL/well), and absorption measured on a GloMax plate reader (Promega) at 450 nM.

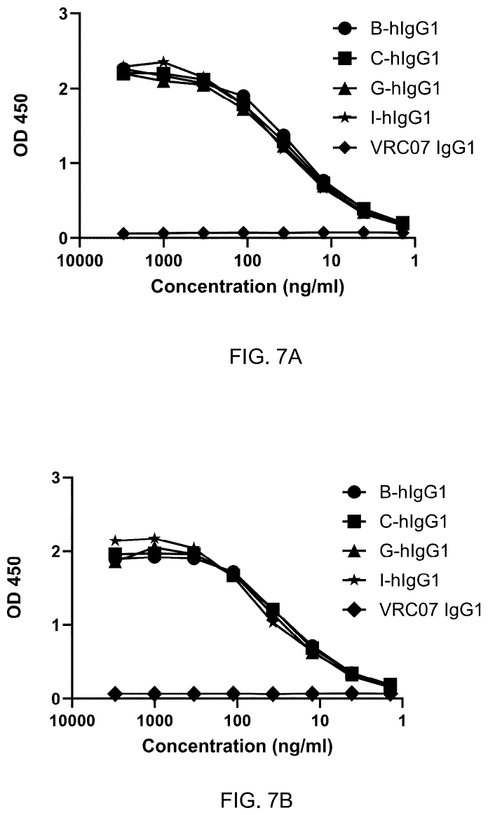

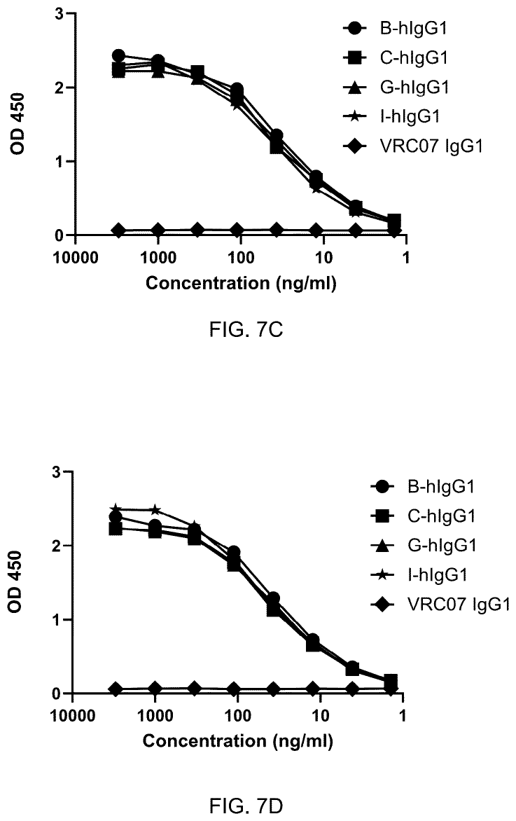

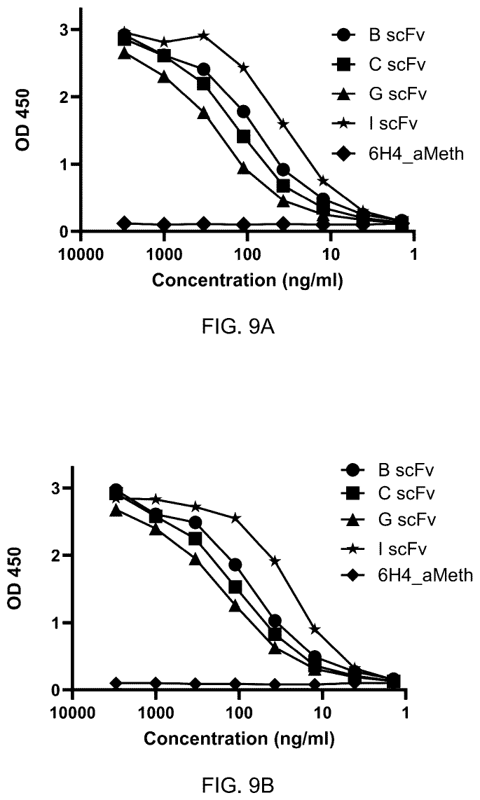

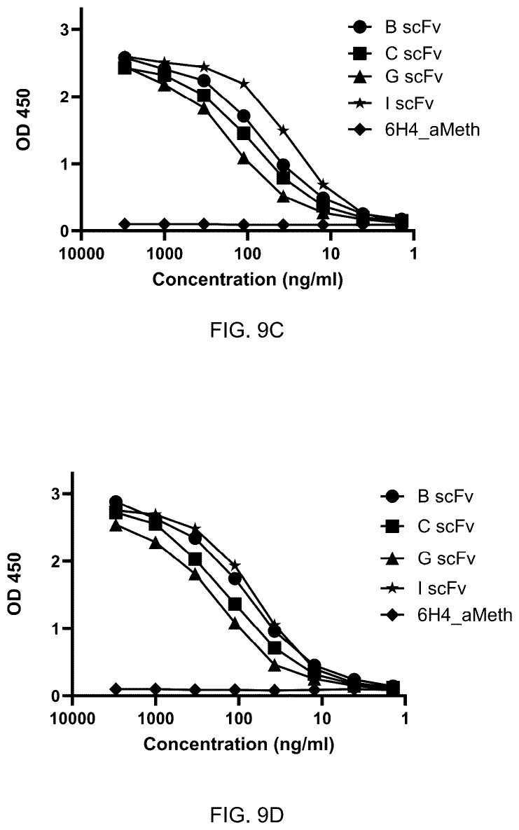

FIGS. 7A-7D show that clones B, C, G, and I in full-length human IgG₁ format are able to bind non-phosphorylated (FIG. 7A), phosphorylated S409 (FIG. 7B), phosphorylated S410 (FIG. 7C), and phosphorylated S409/S410 (FIG. 7D)C-terminal TDP-43 peptides. A similar binding profile was observed for clones B, C, G, and I in scFv format (FIGS. 9A-9D).

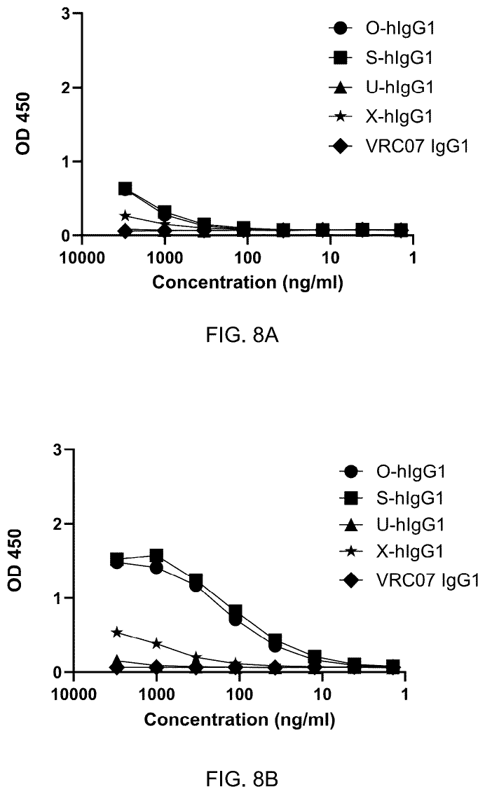

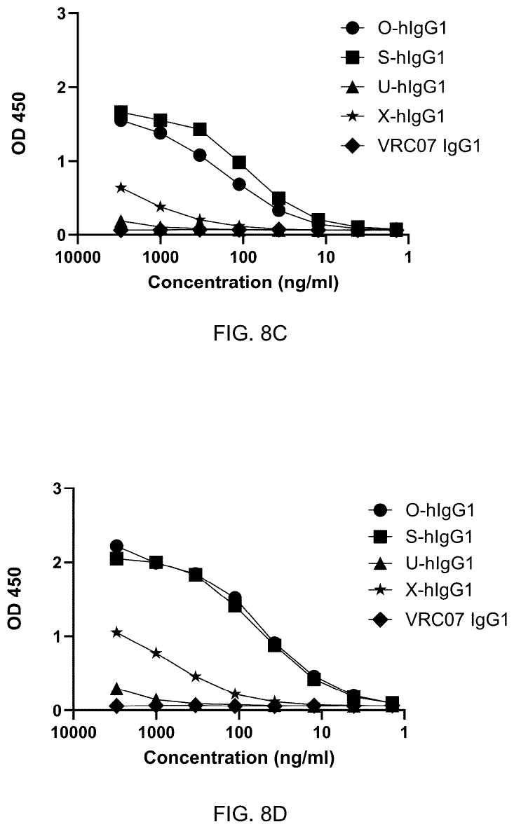

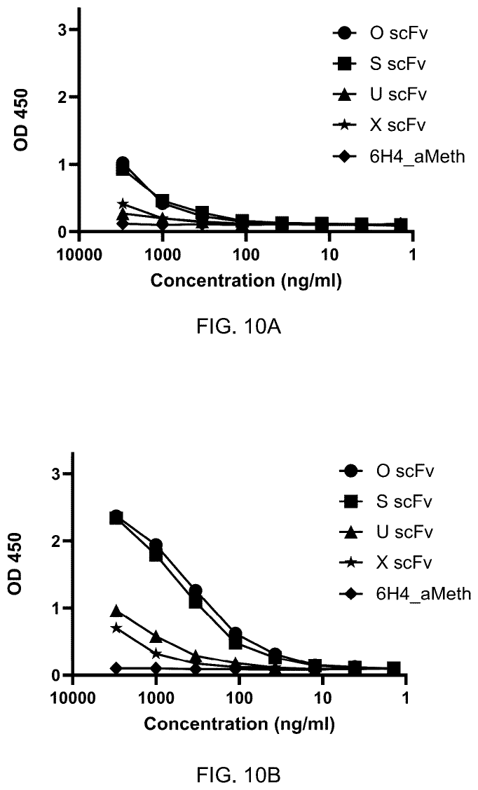

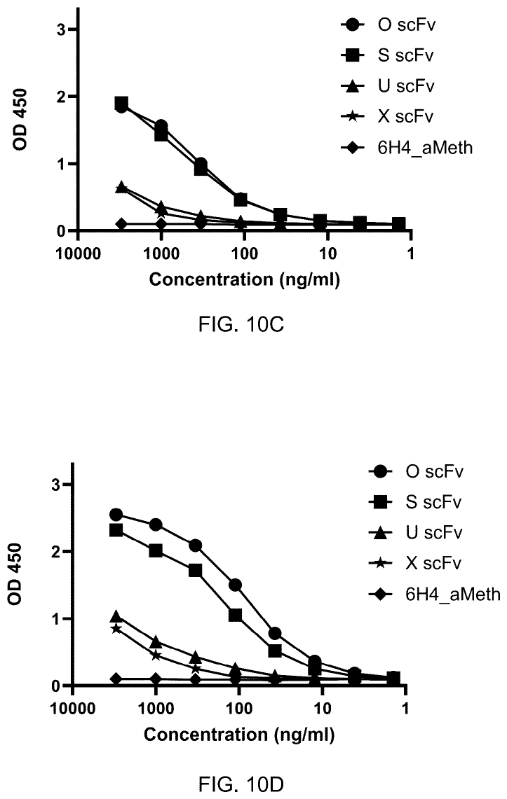

FIGS. 8A-8D show that clones O, S, U, and X in full-length human IgG₁ format are less efficient at binding non-phosphorylated TDP-43 (FIG. 8A). Clones O and S showed preferential binding to phosphorylated S409 (FIG. 8B), phosphorylated S410 (FIG. 8C) and phosphorylated S409/S410 (FIG. 9D)C-terminal TDP-43 peptides. A similar binding profile was observed for clones O, S, U, and X in scFv format (FIGS. 10A-10D).

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. An antibody that specifically binds TDP-43, the antibody comprising a VH comprising CDRH1, CDRH2, and CDRH3 amino acid sequences and/or a VL comprising CDRL1, CDRL2, and CDRL3 amino acid sequences, wherein the antibody comprises the CDRH1, CDRH2, CDRH3, CDRL1, CDRL2, and CDRL3 amino acid sequences of the VH and VL amino acid sequences, respectively, set forth in SEQ ID NOs: 66 and 82; 67 and 83; 68 and 84; 69 and 85; 68 and 86; 68 and 87; 70 and 88; 71 and 89; 72 and 90; 73 and 91; 74 and 92; 75 and 93; 76 and 94; 76 and 95; 77 and 96; 76 and 97; 78 and 98; 76 and 99; 79 and 100; 76 and 101; 76 and 102; 76 and 103; 80 and 104; or 81 and 105.

2. A polypeptide comprising:
(a) the VH or VL of the antibody of claim 1;
(b) the VH and VL of the antibody of claim 1; or
(c) an amino acid sequence selected from the group consisting of SEQ ID NOs: 66-105 and 120-143.

3. A polynucleotide comprising a nucleotide sequence encoding the antibody of claim 1.

4. A polynucleotide comprising a nucleotide sequence encoding an antibody that specifically binds TDP-43, the antibody comprising a VH comprising CDRH1, CDRH2, and CDRH3 amino acid sequences and a VL comprising CDRL1, CDRL2, and CDRL3 amino acid sequences, wherein the antibody comprises the CDRH1, CDRH2, CDRH3, CDRL1, CDRL2, and CDRL3 amino acid sequences of the VH and VL amino acid sequences, respectively, set forth in SEQ ID NOs: 67 and 83, 72 and 90, or 77 and 96.

5. A polynucleotide comprising the nucleotide sequence of any one of SEQ ID NOs: 144-215.

6. The polynucleotide of claim 4, comprising a promoter sequence operably linked to the nucleotide sequence.

7. The polynucleotide of claim 4, wherein the promoter is selected from the group consisting of a Polymerase II promoter, a chicken-beta actin promoter, a CAG promoter, an EF1 alpha promoter, a PGK promoter, and a tissue-specific promoter.

8. The polynucleotide of claim 4, which is a vector.

9. The polynucleotide of claim 8, wherein the vector is a plasmid or a viral vector genome.

10. The polynucleotide of claim 9, wherein the viral vector genome is a recombinant adeno-associated virus (rAAV) vector genome.

11. An rAAV comprising an AAV capsid and the rAAV vector genome of claim 10.

12. The rAAV of claim 11, wherein the AAV capsid comprises a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, AAV5.2, or AAV5 capsid protein, or an engineered variant thereof.

13. The rAAV of claim 11, wherein the AAV capsid comprises a capsid protein comprising the amino acid sequence of SEQ ID NO: 216 or 217.

14. A packaging system for preparation of an rAAV, wherein the packaging system comprises:
(a) a first nucleotide sequence encoding one or more AAV Rep proteins;
(b) a second nucleotide sequence encoding a capsid protein of the rAAV of claim 11; and
(c) a third nucleotide sequence comprising an rAAV genome sequence of the rAAV of claim 11.

15. A method for recombinant preparation of an rAAV, the method comprising introducing the packaging system of claim 14 into a cell under conditions whereby the rAAV is produced.

16. A recombinant host cell comprising the polynucleotide of claim 3.

17. A recombinant host cell comprising the polynucleotide of claim 4.

18. A method of producing an antibody, the method comprising culturing the recombinant host cell of claim 16 under suitable conditions such that the polynucleotide is expressed, and the antibody is produced.

19. A method of producing an antibody, the method comprising culturing the recombinant host cell of claim 17 under suitable conditions such that the polynucleotide is expressed, and the antibody is produced.

20. A composition comprising the antibody of claim 1, or a polynucleotide encoding the antibody of claim 1, and a pharmaceutically acceptable carrier or excipient.

21. A composition comprising the polynucleotide of claim 4.

22. A method of inhibiting an activity of TDP-43 in a subject, the method comprising administering to the subject an effective amount of the polynucleotide of claim 4, or a composition comprising the polynucleotide of claim 4, optionally wherein the composition is administered intravenously, intrathecally, intracisternally, intraparenchymally, intravitreally, or subretinally.

23. A method of reducing TDP-43 protein aggregates in a subject, the method comprising administering to the subject an effective amount of the polynucleotide of claim 4, or a composition comprising the polynucleotide of claim 4, optionally wherein the composition is administered intravenously, intrathecally, intracisternally, intraparenchymally, intravitreally, or subretinally.

24. A method of treating a disease or disorder associated with TDP-43 in a subject, the method comprising administering to the subject an effective amount of the polynucleotide of claim 4, or a composition comprising the polynucleotide of claim 4, optionally wherein the composition is administered intravenously, intrathecally, intracisternally, intraparenchymally, intravitreally, or subretinally.


SEQUENCE LISTING (221 sequences)

SEQ ID NO: 1 - protein, 5 aa, synthetic construct
  REGION 1..5: note = Synthetic Construct
GKYIH

SEQ ID NO: 2 - protein, 17 aa, synthetic construct
  REGION 1..17: note = Synthetic Construct
GINPNNGGTSYNQKFQG

SEQ ID NO: 3 - protein, 16 aa, synthetic construct
  REGION 1..16: note = Synthetic Construct
RSSKSLLHSNGNTYLY

SEQ ID NO: 4 - protein, 7 aa, synthetic construct
  REGION 1..7: note = Synthetic Construct
RMSNLAS

SEQ ID NO: 5 - protein, 9 aa, synthetic construct
  REGION 1..9: note = Synthetic Construct
MQGLQTPLT

SEQ ID NO: 6 - protein, 5 aa, synthetic construct
  REGION 1..5: note = Synthetic Construct
ELSMH

SEQ ID NO: 7 - protein, 16 aa, synthetic construct
  REGION 1..16: note = Synthetic Construct
KSSQSLLHSDGKTYLN

SEQ ID NO: 8 - protein, 7 aa, synthetic construct
  REGION 1..7: note = Synthetic Construct
LASRRAS

SEQ ID NO: 9 - protein, 9 aa, synthetic construct
  REGION 1..9: note = Synthetic Construct
FQGTHFPHT

SEQ ID NO: 10 - protein, 5 aa, synthetic construct
  REGION 1..5: note = Synthetic Construct
EYSMH

SEQ ID NO: 11 - protein, 7 aa, synthetic construct
  REGION 1..7: note = Synthetic Construct
LGSNRAS

SEQ ID NO: 12 - protein, 17 aa, synthetic construct
  REGION 1..17: note = Synthetic Construct
GINPNNGGTRYNQKFQG

SEQ ID NO: 13 - protein, 7 aa, synthetic construct
  REGION 1..7: note = Synthetic Construct
LVSNRAS

SEQ ID NO: 14 - protein, 9 aa, synthetic construct
  REGION 1..9: note = Synthetic Construct
HQGTHFPHT

SEQ ID NO: 15 - protein, 7 aa, synthetic construct
  REGION 1..7: note = Synthetic Construct
LGSSRAS

SEQ ID NO: 16 - protein, 9 aa, synthetic construct
  REGION 1..9: note = Synthetic Construct
MQGTHFPHT

SEQ ID NO: 17 - protein, 16 aa, synthetic construct
  REGION 1..16: note = Synthetic Construct
KSSQSLLHNDGKTYLN

SEQ ID NO: 18 - protein, 7 aa, synthetic construct
  REGION 1..7: note = Synthetic Construct
LASNRAS

SEQ ID NO: 19 - protein, 9 aa, synthetic construct
  REGION 1..9: note = Synthetic Construct
GQGTHFPHT

SEQ ID NO: 20 - protein, 7 aa, synthetic construct
  REGION 1..7: note = Synthetic Construct
LVSKLKS

SEQ ID NO: 21 - protein, 16 aa, synthetic construct
  REGION 1..16: note = Synthetic Construct
KSSQSLLHKDGKTYLN

SEQ ID NO: 22 - protein, 7 aa, synthetic construct
  REGION 1..7: note = Synthetic Construct
LVSKLPS

SEQ ID NO: 23 - protein, 17 aa, synthetic construct
  REGION 1..17: note = Synthetic Construct
GINPNNGGTIYNQKFQG

SEQ ID NO: 24 - protein, 5 aa, synthetic construct
  REGION 1..5: note = Synthetic Construct
SYAIS

SEQ ID NO: 25 - protein, 17 aa, synthetic construct
  REGION 1..17: note = Synthetic Construct
EINPSNGRTVYNQKFQG

SEQ ID NO: 26 - protein, 4 aa, synthetic construct
  REGION 1..4: note = Synthetic Construct
YKDY

SEQ ID NO: 27 - protein, 16 aa, synthetic construct
  REGION 1..16: note = Synthetic Construct
RSSQSIVTAIGNTYLE

SEQ ID NO: 28 - protein, 7 aa, synthetic construct
  REGION 1..7: note = Synthetic Construct
KVSNRFS

SEQ ID NO: 29 - protein, 9 aa, synthetic construct
  REGION 1..9: note = Synthetic Construct
FQGSHVPFT

SEQ ID NO: 30 - protein, 5 aa, synthetic construct
  REGION 1..5: note = Synthetic Construct
SYAIH

SEQ ID NO: 31 - protein, 17 aa, synthetic construct
  REGION 1..17: note = Synthetic Construct
EINPSNGRTNYNQKFQG

SEQ ID NO: 32 - protein, 4 aa, synthetic construct
  REGION 1..4: note = Synthetic Construct
RMDY

SEQ ID NO: 33 - protein, 7 aa, synthetic construct
  REGION 1..7: note = Synthetic Construct
RMSNRAS

SEQ ID NO: 34 - protein, 9 aa, synthetic construct
  REGION 1..9: note = Synthetic Construct
MQALQTPFT

SEQ ID NO: 35 - protein, 5 aa, synthetic construct
  REGION 1..5: note = Synthetic Construct
KYWMH

SEQ ID NO: 36 - protein, 17 aa, synthetic construct
  REGION 1..17: note = Synthetic Construct
SMNPNSGHTGFAQKFQG

SEQ ID NO: 37 - protein, 4 aa, synthetic construct
  REGION 1..4: note = Synthetic Construct
AMDY

SEQ ID NO: 38 - protein, 16 aa, synthetic construct
  REGION 1..16: note = Synthetic Construct
KSSQSLLHSDGKTYLY

SEQ ID NO: 39 - protein, 7 aa, synthetic construct
  REGION 1..7: note = Synthetic Construct
KVSNRFK

SEQ ID NO: 40 - protein, 9 aa, synthetic construct
  REGION 1..9: note = Synthetic Construct
MQKIQLPVT

SEQ ID NO: 41 - protein, 4 aa, synthetic construct
  REGION 1..4: note = Synthetic Construct
YMDY

SEQ ID NO: 42 - protein, 16 aa, synthetic construct
  REGION 1..16: note = Synthetic Construct
RSSESIVHSNGNTYLE

SEQ ID NO: 43 - protein, 7 aa, synthetic construct
  REGION 1..7: note = Synthetic Construct
KVSNKFS

SEQ ID NO: 44 - protein, 9 aa, synthetic construct
  REGION 1..9: note = Synthetic Construct
MQYTHWPPT

SEQ ID NO: 45 - protein, 16 aa, synthetic construct
  REGION 1..16: note = Synthetic Construct
RSSQSLLHSNGYNYLD

SEQ ID NO: 46 - protein, 7 aa, synthetic construct
  REGION 1..7: note = Synthetic Construct
KVENRFS

SEQ ID NO: 47 - protein, 9 aa, synthetic construct
  REGION 1..9: note = Synthetic Construct
MQHTHWPPT

SEQ ID NO: 48 - protein, 17 aa, synthetic construct
  REGION 1..17: note = Synthetic Construct
EINPKNGRTNYNQKFQG

SEQ ID NO: 49 - protein, 9 aa, synthetic construct
  REGION 1..9: note = Synthetic Construct
LQHTHWPPT

SEQ ID NO: 50 - protein, 7 aa, synthetic construct
  REGION 1..7: note = Synthetic Construct
EVSNRFS

SEQ ID NO: 51 - protein, 17 aa, synthetic construct
  REGION 1..17: note = Synthetic Construct
EINPQNGRTNYNQKFQG

SEQ ID NO: 52 - protein, 16 aa, synthetic construct
  REGION 1..16: note = Synthetic Construct
RSSRSLLHSNGYNYLD

SEQ ID NO: 53 - protein, 7 aa, synthetic construct
  REGION 1..7: note = Synthetic Construct
AASSLQS

SEQ ID NO: 54 - protein, 17 aa, synthetic construct
  REGION 1..17: note = Synthetic Construct
EINPSNGRTNYPQKFQG

SEQ ID NO: 55 - protein, 7 aa, synthetic construct
  REGION 1..7: note = Synthetic Construct
KVSNRFT

SEQ ID NO: 56 - protein, 9 aa, synthetic construct
  REGION 1..9: note = Synthetic Construct
MQHTHWPPA

SEQ ID NO: 57 - protein, 7 aa, synthetic construct
  REGION 1..7: note = Synthetic Construct
KSSNRFS

SEQ ID NO: 58 - protein, 16 aa, synthetic construct
  REGION 1..16: note = Synthetic Construct
RSSQSLLNSNGVTFVE

SEQ ID NO: 59 - protein, 7 aa, synthetic construct
  REGION 1..7: note = Synthetic Construct
EVSNRFW

SEQ ID NO: 60 - protein, 17 aa, synthetic construct
  REGION 1..17: note = Synthetic Construct
GINPSNGRTNYNQKFQG

SEQ ID NO: 61 - protein, 4 aa, synthetic construct
  REGION 1..4: note = Synthetic Construct
YMQY

SEQ ID NO: 62 - protein, 7 aa, synthetic construct
  REGION 1..7: note = Synthetic Construct
KVSNQSS

SEQ ID NO: 63 - protein, 9 aa, synthetic construct
  REGION 1..9: note = Synthetic Construct
MQALQTPLT

SEQ ID NO: 64 - protein, 5 aa, synthetic construct
  REGION 1..5: note = Synthetic Construct
SHYLH

SEQ ID NO: 65 - protein, 17 aa, synthetic construct
  REGION 1..17: note = Synthetic Construct
IINPSNGRTNYNQKFQG

SEQ ID NO: 66 - protein, 111 aa, synthetic construct
  REGION 1..111: note = Synthetic Construct
QVQLVQSGAEVKKPGASVKVSCKASGYAFTGKYIHWVRQAPGQGLEWMGGINPNNGGTSYNQKFQGRVTMTRDTSTSTVYMELSSLRSEDTAVYYCARKSWGQGTLVTVSS

SEQ ID NO: 67 - protein, 111 aa, synthetic construct
  REGION 1..111: note = Synthetic Construct
QVQLVQSGAEVKKPGASVKVSCKASGFTFTELSMHWVRQAPGQGLEWMGGINPNNGGTSYNQKFQGRVTMTRDTSTSTVYMELSSLRSEDTAVYYCARESWGQGTLVTVSS

SEQ ID NO: 68 - protein, 111 aa, synthetic construct
  REGION 1..111: note = Synthetic Construct
QVQLVQSGAEVKKPGASVKVSCKASGFTFTEYSMHWVRQAPGQGLEWMGGINPNNGGTSYNQKFQGRVTMTRDTSTSTVYMELSSLRSEDTAVYYCARESWGQGTLVTVSS

SEQ ID NO: 69 - protein, 111 aa, synthetic construct
  REGION 1..111: note = Synthetic Construct
QVQLVQSGAEVKKPGASVKVSCKASGGTFTEYSMHWVRQAPGQGLEWMGGINPNNGGTRYNQKFQGRVTMTRDTSTSTVYMELSSLRSEDTAVYYCARESWGQGTLVTVSS

SEQ ID NO: 70 - protein, 111 aa, synthetic construct
  REGION 1..111: note = Synthetic Construct
QVQLVQSGAEVKKPGASVKVSCKASGFTFTEYSMHWVRQAPGQGLEWMGGINPNNGGTSYNQKFQGRVTMTRDTSTSTVYMELSSLRSEDTAVYYCASESWGQGTLVTVSS

SEQ ID NO: 71 - protein, 111 aa, synthetic construct
  REGION 1..111: note = Synthetic Construct
QVQLVQSGAEVKKPGASVKVSCKASGFTFTEYSMHWVRQAPGQGLEWMGGINPNNGGTRYNQKFQGRVTMTRDTSTSTVYMELSSLRSEDTAVYYCARESWGQGTLVTVSS

SEQ ID NO: 72 - protein, 111 aa, synthetic construct
  REGION 1..111: note = Synthetic Construct
QVQLVQSGAEVKKPGASVKVSCKASGFTFTEYSMHWVRQAPGQGLEWMGGINPNNGGTIYNQKFQGRVTMTRDTSTSTVYMELSSLRSEDTAVYYCARESWGQGTLVTVSS

SEQ ID NO: 73 - protein, 113 aa, synthetic construct
  REGION 1..113: note = Synthetic Construct
QVQLVQSGAEVKKPGASVKVSCKASGGTFSSYAISWVRQAPGQGLEWMGEINPSNGRTVYNQKFQGRVTMTRDTSTSTVYMELSSLRSEDTAVYYCARYKDYWGQGTLVTVSS

SEQ ID NO: 74 - protein, 113 aa, synthetic construct
  REGION 1..113: note = Synthetic Construct
QVQLVQSGAEVKKPGASVKVSCKASGGTFSSYAIHWVRQAPGQGLEWMGEINPSNGRTNYNQKFQGRVTMTRDTSTSTVYMELSSLRSEDTAVYYCARRMDYWGQGTLVTVSS

SEQ ID NO: 75 - protein, 113 aa, synthetic construct
  REGION 1..113: note = Synthetic Construct
QVQLVQSGAEVKKPGASVKVSCKASGYTFTKYWMHWVRQAPGQGLEWVGSMNPNSGHTGFAQKFQGRVTMTRDTSTSTVYMELSSLRSEDTAVYYCARAMDYWGQGTLVTVSS

SEQ ID NO: 76 - protein, 113 aa, synthetic construct
  REGION 1..113: note = Synthetic Construct
QVQLVQSGAEVKKPGASVKVSCKASGYTFTKYWMHWVRQAPGQGLEWMGEINPSNGRTNYNQKFQGRVTMTRDTSTSTVYMELSSLRSEDTAVYYCWRYMDYWGQGTLVTVSS

SEQ ID NO: 77 - protein, 113 aa, synthetic construct
  REGION 1..113: note = Synthetic Construct
QVQLVQSGAEVKKPGASVKVSCKASGYTFTKYWMHWVRQAPGQGLEWMGEINPKNGRTNYNQKFQGRVTMTRDTSTSTVYMELSSLRSEDTAVYYCWRYMDYWGQGTLVTVSS

SEQ ID NO: 78 - protein, 113 aa, synthetic construct
  REGION 1..113: note = Synthetic Construct
QVQLVQSGAEVKKPGASVKVSCKASGYTFTKYWMHWVRQAPGQGLEWMGEINPQNGRTNYNQKFQGRVTMTRDTSTSTVYMELSSLRSEDTAVYYCWRYMDYWGQGTLVTVSS

SEQ ID NO: 79 - protein, 113 aa, synthetic construct
  REGION 1..113: note = Synthetic Construct
QVQLVQSGAEVKKPGASVKVSCKASGYTFTKYWMHWVRQAPGQGLEWMGEINPSNGRTNYPQKFQGRVTMTRDTSTSTVYMELSSLRSEDTAVYYCWRYMDYWGQGTLVTVSS

SEQ ID NO: 80 - protein, 113 aa, synthetic construct
  REGION 1..113: note = Synthetic Construct
QVQLVQSGAEVKKPGASVKVSCKASGYTFTKYWMHWVRQAPGQGLEWMGGINPSNGRTNYNQKFQGRVTMTRDTSTSTVYMELSSLRSEDTAVYYCARYMQYWGQGTLVTVSS

SEQ ID NO: 81 - protein, 113 aa, synthetic construct
  REGION 1..113: note = Synthetic Construct
QVQLVQSGAEVKKPGASVKVSCKASGYTFTSHYLHWVRQAPGQGLEWMGIINPSNGRTNYNQKFQGRVTMTRDTSTSTVYMELSSLRSEDTAVYYCWRYMDYWGQGTLVTVSS

SEQ ID NO: 82 - protein, 112 aa, synthetic construct
  REGION 1..112: note = Synthetic Construct
DIVMTQSPLSLPVTPGEPASISCRSSKSLLHSNGNTYLYWYLQKPGQSPQLLIYRMSNLASGVPDRFSGSGSGTDFTLKISRVEAEDVGVYYCMQGLQTPLTFGGGTKVEIK

SEQ ID NO: 83 - protein, 112 aa, synthetic construct
  REGION 1..112: note = Synthetic Construct
DIVMTQSPLSLPVTPGEPASISCKSSQSLLHSDGKTYLNWYLQKPGQSPQLLIYLASRRASGVPDRFSGSGSGTDFTLKISRVEAEDVGVYYCFQGTHFPHTFGGGTKVEIK

SEQ ID NO: 84 - protein, 112 aa, synthetic construct
  REGION 1..112: note = Synthetic Construct
DIVMTQSPLSLPVTPGEPASISCKSSQSLLHSDGKTYLNWYLQKPGQSPQLLIYLGSNRASGVPDRFSGSGSGTDFTLKISRVEAEDVGVYYCFQGTHFPHTFGGGTKVEIK

SEQ ID NO: 85 - protein, 112 aa, synthetic construct
  REGION 1..112: note = Synthetic Construct
DIVMTQSPLSLPVTPGEPASISCKSSQSLLHSDGKTYLNWYLQKPGQSPQLLIYLVSNRASGVPDRFSGSGSGTDFTLKISRVEAEDVGVYYCHQGTHFPHTFGGGTKVEIK

SEQ ID NO: 86 - protein, 112 aa, synthetic construct
  REGION 1..112: note = Synthetic Construct
DIVMTQSPLSLPVTPGEPASISCKSSQSLLHSDGKTYLNWYLQKPGQSPQLLIYLGSSRASGVPDRFSGSGSGTDFTLKISRVEAEDVGVYYCMQGTHFPHTFGGGTKVEIK

SEQ ID NO: 87 - protein, 112 aa, synthetic construct
  REGION 1..112: note = Synthetic Construct
DIVMTQSPLSLPVTPGEPASISCKSSQSLLHNDGKTYLNWYLQKPGQSPQLLIYLASNRASGVPDRFSGSGSGTDFTLKISRVEAEDVGVYYCGQGTHFPHTFGGGTKVEIK

SEQ ID NO: 88 - protein, 112 aa, synthetic construct
  REGION 1..112: note = Synthetic Construct
DIVMTQSPLSLPVTPGEPASISCKSSQSLLHSDGKTYLNWYLQKPGQSPQLLIYLVSKLKSGVPDRFSGSGSGTDFTLKISRVEAEDVGVYYCMQGTHFPHTFGGGTKVEIK

SEQ ID NO: 89 - protein, 112 aa, synthetic construct
  REGION 1..112: note = Synthetic Construct
DIVMTQSPLSLPVTPGEPASISCKSSQSLLHKDGKTYLNWYLQKPGQSPQLLIYLVSKLPSGVPDRFSGSGSGTDFTLKISRVEAEDVGVYYCMQGTHFPHTFGGGTKVEIK

SEQ ID NO: 90 - protein, 112 aa, synthetic construct
  REGION 1..112: note = Synthetic Construct
DIVMTQSPLSLPVTPGEPASISCKSSQSLLHSDGKTYLNWYLQKPGQSPQLLIYLGSNRASGVPDRFSGSGSGTDFTLKISRVEAEDVGVYYCMQGTHFPHTFGGGTKVEIK

SEQ ID NO: 91 - protein, 112 aa, synthetic construct
  REGION 1..112: note = Synthetic Construct
DIVMTQSPLSLPVTPGEPASISCRSSQSIVTAIGNTYLEWYLQKPGQSPQLLIYKVSNRFSGVPDRFSGSGSGTDFTLKISRVEAEDVGVYYCFQGSHVPFTFGGGTKVEIK

SEQ ID NO: 92 - protein, 112 aa, synthetic construct
  REGION 1..112: note = Synthetic Construct
DIVMTQSPLSLPVTPGEPASISCRSSKSLLHSNGNTYLYWYLQKPGQSPQLLIYRMSNRASGVPDRFSGSGSGTDFTLKISRVEAEDVGVYYCMQALQTPFTFGGGTKVEIK

SEQ ID NO: 93 - protein, 112 aa, synthetic construct
  REGION 1..112: note = Synthetic Construct
DIVMTQSPLSLPVTPGEPASISCKSSQSLLHSDGKTYLYWYLQKPGQSPQLLIYKVSNRFKGVPDRFSGSGSGTDFTLKISRVEAEDVGVYYCMQKIQLPVTFGGGTKVEIK

SEQ ID NO: 94 - protein, 112 aa, synthetic construct
  REGION 1..112: note = Synthetic Construct
DIVMTQSPLSLPVTPGEPASISCRSSESIVHSNGNTYLEWYLQKPGQSPQLLIYKVSNKFSGVPDRFSGSGSGTDFTLKISRVEAEDVGVYYCMQYTHWPPTFGGGTKVEIK

SEQ ID NO: 95 - protein, 112 aa, synthetic construct
  REGION 1..112: note = Synthetic Construct
DIVMTQSPLSLPVTPGEPASISCRSSQSLLHSNGYNYLDWYLQKPGQSPQLLIYKVENRFSGVPDRFSGSGSGTDFTLKISRVEAEDVGVYYCMQHTHWPPTFGGGTKVEIK

SEQ ID NO: 96 - protein, 112 aa, synthetic construct
  REGION 1..112: note = Synthetic Construct
DIVMTQSPLSLPVTPGEPASISCRSSQSLLHSNGYNYLDWYLQKPGQSPQLLIYKVSNRFSGVPDRFSGSGSGTDFTLKISRVEAEDVGVYYCLQHTHWPPTFGGGTKVEIK

SEQ ID NO: 97 - protein, 112 aa, synthetic construct
  REGION 1..112: note = Synthetic Construct
DIVMTQSPLSLPVTPGEPASISCRSSQSLLHSNGYNYLDWYLQKPGQSPQLLIYEVSNRFSGVPDRFSGSGSGTDFTLKISRVEAEDVGVYYCMQHTHWPPTFGGGTKVEIK

SEQ ID NO: 98 - protein, 112 aa, synthetic construct
  REGION 1..112: note = Synthetic Construct
DIVMTQSPLSLPVTPGEPASISCRSSRSLLHSNGYNYLDWYLQKPGQSPQLLIYAASSLQSGVPDRFSGSGSGTDFTLKISRVEAEDVGVYYCLQHTHWPPTFGGGTKVEIK

SEQ ID NO: 99 - protein, 112 aa, synthetic construct
  REGION 1..112: note = Synthetic Construct
DIVMTQSPLSLPVTPGEPASISCRSSQSLLHSNGYNYLDWYLQKPGQSPQLLIYKVSNRFSGVPDRFSGSGSGTDFTLKISRVEAEDVGVYYCMQHTHWPPTFGGGTKVEIK

SEQ ID NO: 100 - protein, 112 aa, synthetic construct
  REGION 1..112: note = Synthetic Construct
DIVMTQSPLSLPVTPGEPASISCRSSQSLLHSNGYNYLDWYLQKPGQSPQLLIYKVSNRFTGVPDRFSGSGSGTDFTLKISRVEAEDVGVYYCMQHTHWPPAFGGGTKVEIK

SEQ ID NO: 101 - protein, 112 aa, synthetic construct
  REGION 1..112: note = Synthetic Construct
DIVMTQSPLSLPVTPGEPASISCRSSQSLLHSNGYNYLDWYLQKPGQSPQLLIYKSSNRFSGVPDRFSGSGSGTDFTLKISRVEAEDVGVYYCMQHTHWPPTFGGGTKVEIK

SEQ ID NO: 102 - protein, 112 aa, synthetic construct
  REGION 1..112: note = Synthetic Construct
DIVMTQSPLSLPVTPGEPASISCRSSQSLLNSNGVTFVEWYLQKPGQSPQLLIYEVSNRFSGVPDRFSGSGSGTDFTLKISRVEAEDVGVYYCLQHTHWPPTFGGGTKVEIK

SEQ ID NO: 103 - protein, 112 aa, synthetic construct
  REGION 1..112: note = Synthetic Construct
DIVMTQSPLSLPVTPGEPASISCRSSQSLLHSNGYNYLDWYLQKPGQSPQLLIYEVSNRFWGVPDRFSGSGSGTDFTLKISRVEAEDVGVYYCLQHTHWPPTFGGGTKVEIK

SEQ ID NO: 104 - protein, 112 aa, synthetic construct
  REGION 1..112: note = Synthetic Construct
DIVMTQSPLSLPVTPGEPASISCRSSKSLLHSNGNTYLYWYLQKPGQSPQLLIYKVSNQSSGVPDRFSGSGSGTDFTLKISRVEAEDVGVYYCMQALQTPLTFGGGTKVEIK

SEQ ID NO: 105 - protein, 112 aa, synthetic construct
  REGION 1..112: note = Synthetic Construct
DIVMTQSPLSLPVTPGEPASISCRSSQSLLHSNGYNYLDWYLQKPGQSPQLLIYEVSNRFSGVPDRFSGSGSGTDFTLKISRVEAEDVGVYYCLQHTHWPPTFGGGTKVEIK

SEQ ID NO: 106 - protein, 7 aa, synthetic construct
  REGION 1..7: note = Synthetic Construct
GSGSGSS

SEQ ID NO: 107 - protein, 5 aa, synthetic construct
  REGION 1..5: note = Synthetic Construct
GSGSS

SEQ ID NO: 108 - skipped

SEQ ID NO: 109 - protein, 12 aa, synthetic construct
  REGION 1..12: note = Synthetic Construct
AAAYPYDVPDYA

SEQ ID NO: 110 - protein, 5 aa, synthetic construct
  REGION 1..5: note = Synthetic Construct
GSGSG

SEQ ID NO: 111 - protein, 10 aa, synthetic construct
  REGION 1..10: note = Synthetic Construct
GSGSGGSGSG

SEQ ID NO: 112 - protein, 20 aa, synthetic construct
  REGION 1..20: note = Synthetic Construct
GSGSGGSGSGGSGSGGSGSG

SEQ ID NO: 113 - protein, 10 aa, synthetic construct
  REGION 1..10: note = Synthetic Construct
LEGGGGSAAA

SEQ ID NO: 114 - skipped

SEQ ID NO: 115 - protein, 5 aa, synthetic construct
  REGION 1..5: note = Synthetic Construct
GGSGS

SEQ ID NO: 116 - protein, 5 aa, synthetic construct
  REGION 1..5: note = Synthetic Construct
GGGGS

SEQ ID NO: 117 - protein, 15 aa, synthetic construct
  REGION 1..15: note = Synthetic Construct
GGGGSGGGGSGGGGS

SEQ ID NO: 118 - protein, 11 aa, synthetic construct
  REGION 1..11: note = Synthetic Construct
EQKLISEEDLS

SEQ ID NO: 119 - protein, 414 aa, synthetic construct
  REGION 1..414: note = Synthetic Construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 120 - protein, 238 aa, synthetic construct
  REGION 1..238: note = Synthetic Construct
QVQLVQSGAEVKKPGASVKVSCKASGYAFTGKYIHWVRQAPGQGLEWMGGINPNNGGTSYNQKFQGRVTMTRDTSTSTVYMELSSLRSEDTAVYYCARKSWGQGTLVTVSSGGGGSGGGGSGGGGSDIVMTQSPLSLPVTPGEPASISCRSSKSLLHSNGNTYLYWYLQKPGQSPQLLIYRMSNLASGVPDRFSGSGSGTDFTLKISRVEAEDVGVYYCMQGLQTPLTFGGGTKVEIK

SEQ ID NO: 121 - protein, 238 aa, synthetic construct
  REGION 1..238: note = Synthetic Construct
QVQLVQSGAEVKKPGASVKVSCKASGFTFTELSMHWVRQAPGQGLEWMGGINPNNGGTSYNQKFQGRVTMTRDTSTSTVYMELSSLRSEDTAVYYCARESWGQGTLVTVSSGGGGSGGGGSGGGGSDIVMTQSPLSLPVTPGEPASISCKSSQSLLHSDGKTYLNWYLQKPGQSPQLLIYLASRRASGVPDRFSGSGSGTDFTLKISRVEAEDVGVYYCFQGTHFPHTFGGGTKVEIK

SEQ ID NO: 122 - protein, 238 aa, synthetic construct
  REGION 1..238: note = Synthetic Construct
QVQLVQSGAEVKKPGASVKVSCKASGFTFTEYSMHWVRQAPGQGLEWMGGINPNNGGTSYNQKFQGRVTMTRDTSTSTVYMELSSLRSEDTAVYYCARESWGQGTLVTVSSGGGGSGGGGSGGGGSDIVMTQSPLSLPVTPGEPASISCKSSQSLLHSDGKTYLNWYLQKPGQSPQLLIYLGSNRASGVPDRFSGSGSGTDFTLKISRVEAEDVGVYYCFQGTHFPHTFGGGTKVEIK

SEQ ID NO: 123 - protein, 238 aa, synthetic construct
  REGION 1..238: note = Synthetic Construct
QVQLVQSGAEVKKPGASVKVSCKASGGTFTEYSMHWVRQAPGQGLEWMGGINPNNGGTRYNQKFQGRVTMTRDTSTSTVYMELSSLRSEDTAVYYCARESWGQGTLVTVSSGGGGSGGGGSGGGGSDIVMTQSPLSLPVTPGEPASISCKSSQSLLHSDGKTYLNWYLQKPGQSPQLLIYLVSNRASGVPDRFSGSGSGTDFTLKISRVEAEDVGVYYCHQGTHFPHTFGGGTKVEIK

SEQ ID NO: 124 - protein, 238 aa, synthetic construct
  REGION 1..238: note = Synthetic Construct
QVQLVQSGAEVKKPGASVKVSCKASGFTFTEYSMHWVRQAPGQGLEWMGGINPNNGGTSYNQKFQGRVTMTRDTSTSTVYMELSSLRSEDTAVYYCARESWGQGTLVTVSSGGGGSGGGGSGGGGSDIVMTQSPLSLPVTPGEPASISCKSSQSLLHSDGKTYLNWYLQKPGQSPQLLIYLGSSRASGVPDRFSGSGSGTDFTLKISRVEAEDVGVYYCMQGTHFPHTFGGGTKVEIK

SEQ ID NO: 125 - protein, 238 aa, synthetic construct
  REGION 1..238: note = Synthetic Construct
QVQLVQSGAEVKKPGASVKVSCKASGFTFTEYSMHWVRQAPGQGLEWMGGINPNNGGTSYNQKFQGRVTMTRDTSTSTVYMELSSLRSEDTAVYYCARESWGQGTLVTVSSGGGGSGGGGSGGGGSDIVMTQSPLSLPVTPGEPASISCKSSQSLLHNDGKTYLNWYLQKPGQSPQLLIYLASNRASGVPDRFSGSGSGTDFTLKISRVEAEDVGVYYCGQGTHFPHTFGGGTKVEIK

SEQ ID NO: 126 - protein, 238 aa, synthetic construct
  REGION 1..238: note = Synthetic Construct
QVQLVQSGAEVKKPGASVKVSCKASGFTFTEYSMHWVRQAPGQGLEWMGGINPNNGGTSYNQKFQGRVTMTRDTSTSTVYMELSSLRSEDTAVYYCASESWGQGTLVTVSSGGGGSGGGGSGGGGSDIVMTQSPLSLPVTPGEPASISCKSSQSLLHSDGKTYLNWYLQKPGQSPQLLIYLVSKLKSGVPDRFSGSGSGTDFTLKISRVEAEDVGVYYCMQGTHFPHTFGGGTKVEIK

SEQ ID NO: 127 - protein, 238 aa, synthetic construct
  REGION 1..238: note = Synthetic Construct
QVQLVQSGAEVKKPGASVKVSCKASGFTFTEYSMHWVRQAPGQGLEWMGGINPNNGGTRYNQKFQGRVTMTRDTSTSTVYMELSSLRSEDTAVYYCARESWGQGTLVTVSSGGGGSGGGGSGGGGSDIVMTQSPLSLPVTPGEPASISCKSSQSLLHKDGKTYLNWYLQKPGQSPQLLIYLVSKLPSGVPDRFSGSGSGTDFTLKISRVEAEDVGVYYCMQGTHFPHTFGGGTKVEIK

SEQ ID NO: 128 - protein, 238 aa, synthetic construct
  REGION 1..238: note = Synthetic Construct
QVQLVQSGAEVKKPGASVKVSCKASGFTFTEYSMHWVRQAPGQGLEWMGGINPNNGGTIYNQKFQGRVTMTRDTSTSTVYMELSSLRSEDTAVYYCARESWGQGTLVTVSSGGGGSGGGGSGGGGSDIVMTQSPLSLPVTPGEPASISCKSSQSLLHSDGKTYLNWYLQKPGQSPQLLIYLGSNRASGVPDRFSGSGSGTDFTLKISRVEAEDVGVYYCMQGTHFPHTFGGGTKVEIK

SEQ ID NO: 129 - protein, 240 aa, synthetic construct
  REGION 1..240: note = Synthetic Construct
QVQLVQSGAEVKKPGASVKVSCKASGGTFSSYAISWVRQAPGQGLEWMGEINPSNGRTVYNQKFQGRVTMTRDTSTSTVYMELSSLRSEDTAVYYCARYKDYWGQGTLVTVSSGGGGSGGGGSGGGGSDIVMTQSPLSLPVTPGEPASISCRSSQSIVTAIGNTYLEWYLQKPGQSPQLLIYKVSNRFSGVPDRFSGSGSGTDFTLKISRVEAEDVGVYYCFQGSHVPFTFGGGTKVEIK

SEQ ID NO: 130 - protein, 240 aa, synthetic construct
  REGION 1..240: note = Synthetic Construct
QVQLVQSGAEVKKPGASVKVSCKASGGTFSSYAIHWVRQAPGQGLEWMGEINPSNGRTNYNQKFQGRVTMTRDTSTSTVYMELSSLRSEDTAVYYCARRMDYWGQGTLVTVSSGGGGSGGGGSGGGGSDIVMTQSPLSLPVTPGEPASISCRSSKSLLHSNGNTYLYWYLQKPGQSPQLLIYRMSNRASGVPDRFSGSGSGTDFTLKISRVEAEDVGVYYCMQALQTPFTFGGGTKVEIK

SEQ ID NO: 131 - protein, 240 aa, synthetic construct
  REGION 1..240: note = Synthetic Construct
QVQLVQSGAEVKKPGASVKVSCKASGYTFTKYWMHWVRQAPGQGLEWVGSMNPNSGHTGFAQKFQGRVTMTRDTSTSTVYMELSSLRSEDTAVYYCARAMDYWGQGTLVTVSSGGGGSGGGGSGGGGSDIVMTQSPLSLPVTPGEPASISCKSSQSLLHSDGKTYLYWYLQKPGQSPQLLIYKVSNRFKGVPDRFSGSGSGTDFTLKISRVEAEDVGVYYCMQKIQLPVTFGGGTKVEIK

SEQ ID NO: 132 - protein, 240 aa, synthetic construct
  REGION 1..240: note = Synthetic Construct
QVQLVQSGAEVKKPGASVKVSCKASGYTFTKYWMHWVRQAPGQGLEWMGEINPSNGRTNYNQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSESIVHSNGNTYLEWYLQKPGQSPQLLIYKVSNKFSGVPDRFSGSGSGTDFTLKISRVEAEDVGVYYCMQYTHWPPTFGGGTKVEIK

SEQ ID NO: 133 - protein, 240 aa, synthetic construct
  REGION 1..240: note = Synthetic Construct
QVQLVQSGAEVKKPGASVKVSCKASGYTFTKYWMHWVRQAPGQGLEWMGEINPSNGRTNYNQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSQSLLHSNGYNYLDWYLQKPGQSPQLLIYKVENRFSGVPDRFSGSGSGTDFTLKISRVEAEDVGVYYCMQHTHWPPTFGGGTKVEIK

SEQ ID NO: 134 - protein, 240 aa, synthetic construct
  REGION 1..240: note = Synthetic Construct
QVQLVQSGAEVKKPGASVKVSCKASGYTFTKYWMHWVRQAPGQGLEWMGEINPKNGRTNYNQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSQSLLHSNGYNYLDWYLQKPGQSPQLLIYKVSNRFSGVPDRFSGSGSGTDFTLKISRVEAEDVGVYYCLQHTHWPPTFGGGTKVEIK

SEQ ID NO: 135 - protein, 240 aa, synthetic construct
  REGION 1..240: note = Synthetic Construct
QVQLVQSGAEVKKPGASVKVSCKASGYTFTKYWMHWVRQAPGQGLEWMGEINPSNGRTNYNQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSQSLLHSNGYNYLDWYLQKPGQSPQLLIYEVSNRFSGVPDRFSGSGSGTDFTLKISRVEAEDVGVYYCMQHTHWPPTFGGGTKVEIK

SEQ ID NO: 136 - protein, 240 aa, synthetic construct
  REGION 1..240: note = Synthetic Construct
QVQLVQSGAEVKKPGASVKVSCKASGYTFTKYWMHWVRQAPGQGLEWMGEINPQNGRTNYNQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSRSLLHSNGYNYLDWYLQKPGQSPQLLIYAASSLQSGVPDRFSGSGSGTDFTLKISRVEAEDVGVYYCLQHTHWPPTFGGGTKVEIK

SEQ ID NO: 137 - protein, 240 aa, synthetic construct
  REGION 1..240: note = Synthetic Construct
QVQLVQSGAEVKKPGASVKVSCKASGYTFTKYWMHWVRQAPGQGLEWMGEINPSNGRTNYNQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSQSLLHSNGYNYLDWYLQKPGQSPQLLIYKVSNRFSGVPDRFSGSGSGTDFTLKISRVEAEDVGVYYCMQHTHWPPTFGGGTKVEIK

SEQ ID NO: 138 - protein, 240 aa, synthetic construct
  REGION 1..240: note = Synthetic Construct
QVQLVQSGAEVKKPGASVKVSCKASGYTFTKYWMHWVRQAPGQGLEWMGEINPSNGRTNYPQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSQSLLHSNGYNYLDWYLQKPGQSPQLLIYKVSNRFTGVPDRFSGSGSGTDFTLKISRVEAEDVGVYYCMQHTHWPPAFGGGTKVEIK

SEQ ID NO: 139 - protein, 240 aa, synthetic construct
  REGION 1..240: note = Synthetic Construct
QVQLVQSGAEVKKPGASVKVSCKASGYTFTKYWMHWVRQAPGQGLEWMGEINPSNGRTNYNQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSQSLLHSNGYNYLDWYLQKPGQSPQLLIYKSSNRFSGVPDRFSGSGSGTDFTLKISRVEAEDVGVYYCMQHTHWPPTFGGGTKVEIK

SEQ ID NO: 140 - protein, 240 aa, synthetic construct
  REGION 1..240: note = Synthetic Construct
QVQLVQSGAEVKKPGASVKVSCKASGYTFTKYWMHWVRQAPGQGLEWMGEINPSNGRTNYNQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSQSLLNSNGVTFVEWYLQKPGQSPQLLIYEVSNRFSGVPDRFSGSGSGTDFTLKISRVEAEDVGVYYCLQHTHWPPTFGGGTKVEIK

SEQ ID NO: 141 - protein, 240 aa, synthetic construct
  REGION 1..240: note = Synthetic Construct
QVQLVQSGAEVKKPGASVKVSCKASGYTFTKYWMHWVRQAPGQGLEWMGEINPSNGRTNYNQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSQSLLHSNGYNYLDWYLQKPGQSPQLLIYEVSNRFWGVPDRFSGSGSGTDFTLKISRVEAEDVGVYYCLQHTHWPPTFGGGTKVEIK

SEQ ID NO: 142 - protein, 240 aa, synthetic construct
  REGION 1..240: note = Synthetic Construct
QVQLVQSGAEVKKPGASVKVSCKASGYTFTKYWMHWVRQAPGQGLEWMGGINPSNGRTNYNQKFQGRVTMTRDTSTSTVYMELSSLRSEDTAVYYCARYMQYWGQGTLVTVSSGGGGSGGGGSGGGGSDIVMTQSPLSLPVTPGEPASISCRSSKSLLHSNGNTYLYWYLQKPGQSPQLLIYKVSNQSSGVPDRFSGSGSGTDFTLKISRVEAEDVGVYYCMQALQTPLTFGGGTKVEIK

SEQ ID NO: 143 - protein, 240 aa, synthetic construct
  REGION 1..240: note = Synthetic Construct
QVQLVQSGAEVKKPGASVKVSCKASGYTFTSHYLHWVRQAPGQGLEWMGIINPSNGRTNYNQKFQGRVTMTRDTSTSTVYMELSSLRSEDTAVYYCWRYMDYWGQGTLVTVSSGGGGSGGGGSGGGGSDIVMTQSPLSLPVTPGEPASISCRSSQSLLHSNGYNYLDWYLQKPGQSPQLLIYEVSNRFSGVPDRFSGSGSGTDFTLKISRVEAEDVGVYYCLQHTHWPPTFGGGTKVEIK

SEQ ID NO: 144 - DNA, 333 bp, synthetic construct
  misc_feature 1..333: note = Synthetic Construct
  source 1..333: mol_type = other DNA
caggtgcagctggtgcagtctggggctgaggtgaagaagcctggggcctcagtgaaggtttcctgcaaggcatctggatacgcattcaccgggaagtatatccactgggtgcgacaggcccctggacaagggcttgagtggatgggaggaatcaacccaaataacggtggcacaagctacaatcagaagttccagggcagagtcaccatgaccagggacacgtccacgagcacagtctacatggagctgagcagcctgagatctgaggacacggccgtgtattactgtgctagaaaatcctggggccaaggaaccctggtcaccgtctcctca

SEQ ID NO: 145 - DNA, 333 bp, synthetic construct
  misc_feature 1..333: note = Synthetic Construct
  source 1..333: mol_type = other DNA
caggtgcagctggtgcagtctggggctgaggtgaagaagcctggggcctcagtgaaggtttcctgcaaggcatctggatttaccttcaccgagctgtccatgcactgggtgcgacaggcccctggacaagggcttgagtggatgggaggaatcaaccctaataacggtggcacaagctacaatcagaagttccagggcagagtcaccatgaccagggacacgtccacgagcacagtctacatggagctgagcagcctgagatctgaggacacggccgtgtattactgtgctagagaatcctggggccaaggaaccctggtcaccgtctcctca

SEQ ID NO: 146 - DNA, 333 bp, synthetic construct
  misc_feature 1..333: note = Synthetic Construct
  source 1..333: mol_type = other DNA
caggtgcagctggtgcagtctggggctgaggtgaagaagcctggggcctcagtgaaggtttcctgcaaggcatctggattcaccttcaccgagtacagtatgcactgggtgcgacaggcccctggacaagggcttgagtggatgggaggcatcaaccctaacaacggtggcacaagctacaaccagaagttccagggcagagtcaccatgaccagggacacgtccacgagcacagtctacatggagctgagcagcctgagatctgaggacacggccgtgtattactgtgctagagagtcctggggccaaggaaccctggtcaccgtctcctca

SEQ ID NO: 147 - DNA, 333 bp, synthetic construct
  misc_feature 1..333: note = Synthetic Construct
  source 1..333: mol_type = other DNA
caggtgcagctggtgcagtctggggctgaggtgaagaagcctggggcctcagtgaaggtttcctgcaaggcatctggaggcaccttcaccgagtactctatgcactgggtgcgacaggcccctggacaagggcttgagtggatgggaggaatcaaccccaataacggtggcacacgctacaaccagaagttccagggcagagtcaccatgaccagggacacgtccacgagcacagtctacatggagctgagcagcctgagatctgaggacacggccgtgtattactgtgctagagaatcctggggccaaggaaccctggtcaccgtctcctca

SEQ ID NO: 148 - DNA, 333 bp, synthetic construct
  misc_feature 1..333: note = Synthetic Construct
  source 1..333: mol_type = other DNA
caggtgcagctggtgcagtctggggctgaggtgaagaagcctggggcctcagtgaaggtttcctgcaaggcatctggattcaccttcaccgagtactccatgcactgggtgcgacaggcccctggacaagggcttgagtggatgggaggaatcaacccaaacaacggtggcacaagctacaaccagaagttccagggcagagtcaccatgaccagggacacgtccacgagcacagtctacatggagctgagcagcctgagatctgaggacacggccgtgtattactgtgctagagaatcctggggccaaggaaccctggtcaccgtctcctca

SEQ ID NO: 149 - DNA, 333 bp, synthetic construct
  misc_feature 1..333: note = Synthetic Construct
  source 1..333: mol_type = other DNA
caggtgcagctggtgcagtctggggctgaggtgaagaagcctggggcctcagtgaaggtttcctgcaaggcatctggattcaccttcaccgaatacagcatgcactgggtgcgacaggcccctggacaagggcttgagtggatgggaggaatcaaccccaataatggtggaacaagctacaatcagaagttccagggcagagtcaccatgaccagggacacgtccacgagcacagtctacatggagctgagcagcctgagatctgaggacacggccgtgtattactgtgctagagaatcctggggccaaggaaccctggtcaccgtctcctca

SEQ ID NO: 150 - DNA, 333 bp, synthetic construct
  misc_feature 1..333: note = Synthetic Construct
  source 1..333: mol_type = other DNA
caggtgcagctggtgcagtctggggctgaggtgaagaagcctggggcctcagtgaaggtttcctgcaaggcatctggattcaccttcaccgagtactcaatgcactgggtgcgacaggcccctggacaagggcttgagtggatgggagggatcaacccaaacaacggtggaacaagctacaatcagaagttccagggcagagtcaccatgaccagggacacgtccacgagcacagtctacatggagctgagcagcctgagatctgaggacacggccgtgtattactgtgcttccgagtcctggggccaaggaaccctggtcaccgtctcctca

SEQ ID NO: 151 - DNA, 333 bp, synthetic construct
  misc_feature 1..333: note = Synthetic Construct
  source 1..333: mol_type = other DNA
caggtgcagctggtgcagtctggggctgaggtgaagaagcctggggcctcagtgaaggtttcctgcaaggcatctggattcaccttcaccgagtactccatgcactgggtgcgacaggcccctggacaagggcttgagtggatgggaggaatcaacccaaacaatggtggaacaaggtacaaccagaagttccagggcagagtcaccatgaccagggacacgtccacgagcacagtctacatggagctgagcagcctgagatctgaggacacggccgtgtattactgtgctagagaatcctggggccaaggaaccctggtcaccgtctcctca

SEQ ID NO: 152 - DNA, 333 bp, synthetic construct
  misc_feature 1..333: note = Synthetic Construct
  source 1..333: mol_type = other DNA
caggtgcagctggtgcagtctggggctgaggtgaagaagcctggggcctcagtgaaggtttcctgcaaggcatctggatttaccttcaccgagtactcgatgcactgggtgcgacaggcccctggacaagggcttgagtggatgggaggcatcaacccaaacaacggtggcacaatctacaaccagaagttccagggcagagtcaccatgaccagggacacgtccacgagcacagtctacatggagctgagcagcctgagatctgaggacacggccgtgtattactgtgctagagagtcctggggccaaggaaccctggtcaccgtctcctca

SEQ ID NO: 153 - DNA, 339 bp, synthetic construct
  misc_feature 1..339: note = Synthetic Construct
  source 1..339: mol_type = other DNA
caagtgcagctggtgcagagcggcgccgaggtgaagaagcccggcgcaagcgtgaaggtgagctgcaaggcaagcggcggcaccttcagcagctacgccatcagctgggtgagacaagcccccggccaaggcctggagtggatgggcgagatcaaccctagcaacggcagaaccgtgtacaatcagaagttccaaggcagagtgaccatgacaagagacacaagcacaagcaccgtgtacatggaactgagcagcctgagaagcgaggacaccgccgtttactactgcgctagatacaaggattattgggggcaaggcaccttggttaccgttagcagc

SEQ ID NO: 154 - DNA, 339 bp, synthetic construct
  misc_feature 1..339: note = Synthetic Construct
  source 1..339: mol_type = other DNA
caagtgcagctggtgcagagcggcgccgaggtgaagaagcccggcgcaagcgtgaaggtgagctgcaaggcaagcggcggcaccttcagcagctacgccatccactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtatattactgcgctagaagaatggactattggggccaaggcaccctggtaacagtatcctcc

SEQ ID NO: 155 - DNA, 339 bp, synthetic construct
  misc_feature 1..339: note = Synthetic Construct
  source 1..339: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtgggtgggcagcatgaaccccaacagcggccacaccggcttcgctcagaagttccaaggcagagtgaccatgacaagagacacaagcacaagcaccgtgtacatggaactgagcagcctgagaagcgaggacaccgccgtatattactgcgctagagctatggactattgggggcaaggcaccctggtgaccgtgtcatcc

SEQ ID NO: 156 - DNA, 339 bp, synthetic construct
  misc_feature 1..339: note = Synthetic Construct
  source 1..339: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtatattactgctggagatacatggactattggggccaaggcaccctggtaacagtatcctcc

SEQ ID NO: 157 - DNA, 339 bp, synthetic construct
  misc_feature 1..339: note = Synthetic Construct
  source 1..339: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtttactactgctggagatacatggattattgggggcaaggcaccttggttaccgttagcagc

SEQ ID NO: 158 - DNA, 339 bp, synthetic construct
  misc_feature 1..339: note = Synthetic Construct
  source 1..339: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccccaagaacggcagaaccaactacaatcagaagttccaaggcagagtgaccatgacaagagacacaagcacaagcaccgtgtacatggaactgagcagcctgagaagcgaggacaccgccgtttactactgctggagatacatggattattgggggcaaggcaccttggttaccgttagcagc

SEQ ID NO: 159 - DNA, 339 bp, synthetic construct; identical to SEQ ID NO: 157
  misc_feature 1..339: note = Synthetic Construct
  source 1..339: mol_type = other DNA

SEQ ID NO: 160 - DNA, 339 bp, synthetic construct
  misc_feature 1..339: note = Synthetic Construct
  source 1..339: mol_type = other DNA
caagtgcagctggtccaaagcggtgcagaggtgaagaagcccggcgcaagcgtgaaggtgagctgcaaggcaagcggctacaccttcaccaagtactggatgcactgggtgagacaagcccccggccaaggcctggagtggatgggcgagatcaaccctcagaacggcagaaccaactacaatcagaagttccaaggcagagtgaccatgacaagagacacaagcacaagcaccgtgtacatggaactgagcagcctgagaagcgaggacaccgccgtatattactgctggagatacatggactattgggggcaaggcaccctggtgaccgtgtcaagc

SEQ ID NO: 161 - DNA, 339 bp, synthetic construct; identical to SEQ ID NO: 157
  misc_feature 1..339: note = Synthetic Construct
  source 1..339: mol_type = other DNA

SEQ ID NO: 162 - DNA, 339 bp, synthetic construct
  misc_feature 1..339: note = Synthetic Construct
  source 1..339: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactaccctcagaagttccaaggcagagtgaccatgacaagagacacaagcacaagcaccgtgtacatggaactgagcagcctgagaagcgaggacaccgccgtatattactgctggagatacatggactattggggccaaggcaccctggtaacagtatcctcc

SEQ ID NO: 163 - DNA, 339 bp, synthetic construct; identical to SEQ ID NO: 157
  misc_feature 1..339: note = Synthetic Construct
  source 1..339: mol_type = other DNA

SEQ ID NO: 164 - DNA, 339 bp, synthetic construct; identical to SEQ ID NO: 157
  misc_feature 1..339: note = Synthetic Construct
  source 1..339: mol_type = other DNA

SEQ ID NO: 165 - DNA, 339 bp, synthetic construct; identical to SEQ ID NO: 156
  misc_feature 1..339: note = Synthetic Construct
  source 1..339: mol_type = other DNA

SEQ ID NO: 166 - DNA, 339 bp, synthetic construct
  misc_feature 1..339: note = Synthetic Construct
  source 1..339: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcggcatcaaccctagcaacggcagaaccaactacaatcagaagttccaaggcagagtgaccatgacaagagacacaagcacaagcaccgtgtacatggaactgagcagcctgagaagcgaggacaccgccgtatattactgcgctagatacatgcagtattggggccaaggcaccctggtaacagtatcctcc

SEQ ID NO: 167 - DNA, 339 bp, synthetic construct
  misc_feature 1..339: note = Synthetic Construct
  source 1..339: mol_type = other DNA
caagtgcagctggtgcagagcggcgccgaggtgaagaagcccggcgcaagcgtgaaggtgagctgcaaggcaagcggctacaccttcacaagccactacctgcactgggtgagacaagcccccggccaaggcctggagtggatgggcatcatcaaccctagcaacggcagaaccaactacaatcagaagttccaaggcagagtgaccatgacaagagacacaagcacaagcaccgtgtacatggaactgagcagcctgagaagcgaggacaccgccgtttactactgctggagatacatggattattgggggcaaggcaccttggttaccgttagcagc

SEQ ID NO: 168 - DNA, 336 bp, synthetic construct
  misc_feature 1..336: note = Synthetic Construct
  source 1..336: mol_type = other DNA
gatattgtgatgactcagtctccactctccctgcccgtcacccctggagagccggcctccatctcctgcaggtctagtaaaagcctcttgcactctaatggaaacacctatttgtattggtacctgcagaagccagggcagtctccacagctcctgatctatcggatgtctaatctcgcctccggggtccctgacaggttcagtggcagtggatcaggcacagattttacactgaaaatcagcagagtggaggctgaggatgttggggtttattactgcatgcaaggactacaaactcctctcactttcggcggagggaccaaggtggagatcaaa

SEQ ID NO: 169 - DNA, 336 bp, synthetic construct
  misc_feature 1..336: note = Synthetic Construct
  source 1..336: mol_type = other DNA
gatattgtgatgacccagtcgccactctctctgcctgtcacccctggagagccggcctccatctcctgcaagtctagtcagagcctcctccactcggatggcaagacctatttgaattggtacctgcagaagccaggccagtctccacagctcctaatctatttagcttcccggcgggcctctggagtgccagataggttcagtggcagcgggtcagggacagatttcacactgaaaatcagccgggtggaggctgaggatgttggggtttattactgctttcaaggtacccactttcctcacactttcggcggagggaccaaggtggagatcaaa

SEQ ID NO: 170 - DNA, 336 bp, synthetic construct
  misc_feature 1..336: note = Synthetic Construct
  source 1..336: mol_type = other DNA
gatattgtgatgactcagtctccactctccctgcccgtcacccctggagagccggcctccatctcctgcaaatctagtcagagcctcctgcattcagatgggaagacatatttgaactggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgctttcaaggcacccatttccctcacactttcggcggagggaccaaggtggagatcaaa

SEQ ID NO: 171 - DNA, 336 bp, synthetic construct
  misc_feature 1..336: note = Synthetic Construct
  source 1..336: mol_type = other DNA
gatattgtgatgacccagtcaccactctctctgccagtcacccctggagagccggcctccatctcctgcaagtctagtcagagcctcctacactccgatggaaagacctatttgaattggtacctgcagaagccaggccagtctccacagctcctaatctatttggtttccaaccgggcctctggagtgccagataggttcagtggcagcgggtcagggacagatttcacactgaaaatcagccgggtggaggctgaggatgttggggtttattactgccaccaaggtactcactttcctcatactttcggcggagggaccaaggtggagatcaaa

SEQ ID NO: 172 - DNA, 336 bp, synthetic construct
  misc_feature 1..336: note = Synthetic Construct
  source 1..336: mol_type = other DNA
gatattgtgatgacccagagcccactctctctgcccgtcacccctggagaaccggcctccatctcctgcaagtctagtcagagcctcctgcactccgatgggaagacctatttgaattggtacctgcagaagccaggccagtctccacagctcctaatctatctgggctccagccgggcctctggagtgccagataggttcagtggcagcgggtcagggacagatttcacactgaaaatcagccgggtggaggctgaggatgttggggtttattactgcatgcaaggtactcactttcctcatactttcggcggagggaccaaggtggagatcaaa

SEQ ID NO: 173 - DNA, 336 bp, synthetic construct
  misc_feature 1..336: note = Synthetic Construct
  source 1..336: mol_type = other DNA
gatattgtgatgactcagtctccactctccctgcccgtcacccctggagagccggcctccatctcctgcaagtctagtcagagcctcctgcacaatgatgggaaaacatatttgaattggtacctgcagaagccagggcagtctccacagctcctgatctatttggcttctaatcgggcctccggggtccctgacaggttcagtggcagtggatcaggcacagattttacactgaaaatcagcagagtggaggctgaggatgttggggtttattactgcggccaaggcacgcatttccctcatactttcggcggagggaccaaggtggagatcaaa

SEQ ID NO: 174 - DNA, 336 bp, synthetic construct
  misc_feature 1..336: note = Synthetic Construct
  source 1..336: mol_type = other DNA
gatattgtgatgacccagtctccactctctctgccggtcacccctggagaaccggcctccatctcctgcaagtctagtcagagcctcctgcactctgacggaaagacctatttgaactggtacctgcagaagccaggccagtctccacagctcctaatctatctggtttccaagctgaagtctggagtgccagataggttcagtggcagcgggtcagggacagatttcacactgaaaatcagccgggtggaggctgaggatgttggggtttattactgcatgcaaggtacccactttcctcatactttcggcggagggaccaaggtggagatcaaa

SEQ ID NO: 175 - DNA, 336 bp, synthetic construct
  misc_feature 1..336: note = Synthetic Construct
  source 1..336: mol_type = other DNA
gatattgtgatgacccagtccccactctctctgcctgtcacccctggagagccggcctccatctcctgcaagtctagtcagagcctccttcacaaggatggtaagacctatttgaactggtacctgcagaagccaggccagtctccacagctcctaatctatctggtttccaaacttccatctggagtgccagataggttcagtggcagcgggtcagggacagatttcacactgaaaatcagccgggtggaggctgaggatgttggggtttattactgcatgcaaggtactcacttccctcatactttcggcggagggaccaaggtggagatcaaa

SEQ ID NO: 176 - DNA, 336 bp, synthetic construct
  misc_feature 1..336: note = Synthetic Construct
  source 1..336: mol_type = other DNA
gatattgtgatgactcagtctccactctccctgcccgtcacccctggagagccggcctccatctcctgcaagtctagtcagagcctcttgcactctgatgggaaaacctatttgaattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaggaacccattttcctcacactttcggcggagggaccaaggtggagatcaaa

SEQ ID NO: 177 - DNA, 336 bp, synthetic construct
  misc_feature 1..336: note = Synthetic Construct
  source 1..336: mol_type = other DNA
gatatcgtgatgacacagagccccctgtcgctgcccgtgactcccggcgaacccgcaagcatcagctgcagaagctctcagagcatcgtgaccgccatcggcaacacctacctggagtggtatctgcagaagcccgggcagagccctcagctgctgatctacaaggtgagcaataggttcagcggcgtgcctgatagattcagcggaagcggtagcggcaccgacttcaccctgaagatcagcagagtggaggccgaggacgtgggcgtgtactattgcttccaaggcagccacgtgcccttcaccttcggcggcggcaccaaggtggagatcaag

SEQ ID NO: 178 - DNA, 336 bp, synthetic construct
  misc_feature 1..336: note = Synthetic Construct
  source 1..336: mol_type = other DNA
gatatcgtgatgacacagagccccctgagcctgcccgtgacccccggcgagcccgcaagcatcagctgcagaagcagcaagagcctgctgcacagcaacggcaacacctacctgtactggtatctgcagaagcccgggcagagccctcagctgctgatctacagaatgagcaacagagcaagcggcgtgcccgacagattcagcggcagcggcagcggcaccgacttcaccctgaagatcagcagagtggaggccgaggacgtgggcgtctattactgtatgcaagccctgcagacccccttcaccttcggcggcggcaccaaggtggagatcaag

SEQ ID NO: 179 - DNA, 336 bp, synthetic construct
  misc_feature 1..336: note = Synthetic Construct
  source 1..336: mol_type = other DNA
gatatcgtgatgacacagagccccctgagcctgcccgtgacccccggcgaacccgcaagcatcagctgcaaaagctctcagagcctgctgcacagcgacggcaagacctacctgtactggtatctgcagaagcccgggcagagccctcagctgctgatctacaaggtgagcaacagattcaagggcgtgcccgacagattcagcggcagcggcagcggcaccgacttcaccctgaagatcagcagagtggaggccgaggacgtgggcgtctattactgtatgcagaagattcagctgcccgtaacatttggcggcggcaccaaggtggagatcaag

SEQ ID NO: 180 - DNA, 336 bp, synthetic construct
  misc_feature 1..336: note = Synthetic Construct
  source 1..336: mol_type = other DNA
gatatcgtgatgacccaaagccccctgagcctacccgtgacccccggcgagcccgcaagcatcagctgcagaagcagcgagagcatcgtgcacagcaacggcaacacctacctggagtggtatctgcagaagcccgggcagagccctcagctgctgatctacaaggtgagcaacaagttcagcggcgtgcccgacagattcagcggcagcggcagcggcaccgacttcaccctgaagatcagcagagtggaggccgaggacgtgggcgtctattactgtatgcagtacacccactggccccccaccttcggcggcggcaccaaggtggagatcaag

SEQ ID NO: 181 - DNA, 336 bp, synthetic construct
  misc_feature 1..336: note = Synthetic Construct
  source 1..336: mol_type = other DNA
gatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacaaggtggagaataggttcagcggcgtgcctgatagattcagcggaagcggtagcggcaccgacttcaccctgaagatcagcagagtggaggccgaggacgtgggcgtgtactattgtatgcagcacacccactggccccccaccttcggcggcggcaccaaggtggagatcaag

SEQ ID NO: 182 - DNA, 336 bp, synthetic construct
  misc_feature 1..336: note = Synthetic Construct
  source 1..336: mol_type = other DNA
gatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacaaggtgagcaataggttcagcggcgtgcctgatagattcagcggaagcggtagcggcaccgacttcaccctgaagatcagcagagtggaggccgaggacgtgggcgtgtactattgcctgcaacacacccactggccccccaccttcggcggcggcaccaaggtggagatcaag

SEQ ID NO: 183 - DNA, 336 bp, synthetic construct
  misc_feature 1..336: note = Synthetic Construct
  source 1..336: mol_type = other DNA
gatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacgaggtgagcaataggttcagcggcgtgcctgatagattcagcggaagcggtagcggcaccgacttcaccctgaagatcagcagagtggaggccgaggacgtgggcgtgtactattgtatgcagcacacccactggccccccaccttcggcggcggcaccaaggtggagatcaag

SEQ ID NO: 184 - DNA, 336 bp, synthetic construct
  misc_feature 1..336: note = Synthetic Construct
  source 1..336: mol_type = other DNA
gatatcgtgatgacacagagccccctgagcctgcccgtgacccccggcgagcccgcaagcatcagctgcagaagcagcagaagcctgctgcacagcaacggctacaactacctggactggtatctgcagaagcccgggcagagccctcagctgctgatctacgccgcaagcagcctgcagtcgggggtgcccgacagattcagcggcagcggcagcggcaccgacttcaccctgaagatcagcagagtggaggccgaggacgtgggcgtctattactgcctgcaacacacccactggccccccaccttcggcggcggcaccaaggtggagatcaag

SEQ ID NO: 185 - DNA, 336 bp, synthetic construct
  misc_feature 1..336: note = Synthetic Construct
  source 1..336: mol_type = other DNA
gatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacaaggtgagcaataggttcagcggcgtgcctgatagattcagcggaagcggtagcggcaccgacttcaccctgaagatcagcagagtggaggccgaggacgtgggcgtgtactattgtatgcagcacacccactggccccccaccttcggcggcggcaccaaggtggagatcaag

SEQ ID NO: 186 - DNA, 336 bp, synthetic construct
  misc_feature 1..336: note = Synthetic Construct
  source 1..336: mol_type = other DNA
gatatcgtgatgacccaaagccccctgagcctacccgtgacccccggcgagcccgcaagcatcagctgcagaagctctcagagcctgctgcacagcaacggctacaactacctggactggtatctgcagaagcccgggcagagccctcagctgctgatctacaaggtgagcaacagattcaccggcgtgcccgacagattcagcggcagcggcagcggcaccgacttcaccctgaagatcagcagagtggaggccgaggacgtgggcgtctattactgtatgcagcacacccactggccccccgccttcggcggcggcaccaaggtggagatcaag

SEQ ID NO: 187 - DNA, 336 bp, synthetic construct
  misc_feature 1..336: note = Synthetic Construct
  source 1..336: mol_type = other DNA
gatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacaagagcagcaataggttcagcggcgtgcctgatagattcagcggaagcggtagcggcaccgacttcaccctgaagatcagcagagtggaggccgaggacgtgggcgtgtactattgtatgcagcacacccactggccccccaccttcggcggcggcaccaaggtggagatcaag

SEQ ID NO: 188 - DNA, 336 bp, synthetic construct
  misc_feature 1..336: note = Synthetic Construct
  source 1..336: mol_type = other DNA
gatatcgtgatgacacagagccccctgtcgctgcccgtgactcccggcgaacccgcaagcatcagctgcagaagctctcagagcctgctgaacagcaacggcgtgaccttcgtggagtggtatctgcagaagcccgggcagagccctcagctgctgatctacgaggtgagcaataggttcagcggcgtgcctgatagattcagcggaagcggtagcggcaccgacttcaccctgaagatcagcagagtggaggccgaggacgtgggcgtgtactattgcctgcaacacacccactggccccccaccttcggcggcggcaccaaggtggagatcaag

SEQ ID NO: 189 - DNA, 336 bp, synthetic construct
  misc_feature 1..336: note = Synthetic Construct
  source 1..336: mol_type = other DNA
gatatcgtgatgacccaaagccccctgagcctacccgtgacccccggcgagcccgcaagcatcagctgcagaagctctcagagcctgctgcacagcaacggctacaactacctggactggtatctgcagaagcccgggcagagccctcagctgctgatctacgaggtgagcaacagattctggggcgtgcccgacagattcagcggcagcggcagcggcaccgacttcaccctgaagatcagcagagtggaggccgaggacgtgggcgtctattactgcctgcaacacacccactggccccccaccttcggcggcggcaccaaggtggagatcaag

SEQ ID NO: 190 - DNA, 336 bp, synthetic construct
  misc_feature 1..336: note = Synthetic Construct
  source 1..336: mol_type = other DNA
gatatcgtgatgacacagagccccctgagcctgcccgtgacccccggcgagcccgcaagcatcagctgcagaagcagcaagagcctgctgcacagcaacggcaacacctacctgtactggtatctgcagaagcccgggcagagccctcagctgctgatctacaaggtgagcaatcagagcagcggcgtgcccgacagattcagcggcagcggcagcggcaccgacttcaccctgaagatcagcagagtggaggccgaggacgtgggcgtctattactgtatgcaagccctgcagacccccctgaccttcggcggcggcaccaaggtggagatcaag

SEQ ID NO: 191 - DNA, 336 bp, synthetic construct
  misc_feature 1..336: note = Synthetic Construct
  source 1..336: mol_type = other DNA
gatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacgaggtgagcaataggttcagcggcgtgcctgatagattcagcggaagcggtagcggcaccgacttcaccctgaagatcagcagagtggaggccgaggacgtgggcgtgtactattgcctgcaacacacccactggccccccaccttcggcggcggcaccaaggtggagatcaag

SEQ ID NO: 192 - DNA, 714 bp, synthetic construct
  misc_feature 1..714: note = Synthetic Construct
  source 1..714: mol_type = other DNA
caggtgcagctggtgcagtctggggctgaggtgaagaagcctggggcctcagtgaaggtttcctgcaaggcatctggatacgcattcaccgggaagtatatccactgggtgcgacaggcccctggacaagggcttgagtggatgggaggaatcaacccaaataacggtggcacaagctacaatcagaagttccagggcagagtcaccatgaccagggacacgtccacgagcacagtctacatggagctgagcagcctgagatctgaggacacggccgtgtattactgtgctagaaaatcctggggccaaggaaccctggtcaccgtctcctcaggtggcggaggatctggcggaggtggaagcggcggaggcggatctgatattgtgatgactcagtctccactctccctgcccgtcacccctggagagccggcctccatctcctgcaggtctagtaaaagcctcttgcactctaatggaaacacctatttgtattggtacctgcagaagccagggcagtctccacagctcctgatctatcggatgtctaatctcgcctccggggtccctgacaggttcagtggcagtggatcaggcacagattttacactgaaaatcagcagagtggaggctgaggatgttggggtttattactgcatgcaaggactacaaactcctctcactttcggcggagggaccaaggtggagatcaaa

SEQ ID NO: 193 - DNA, 714 bp, synthetic construct
  misc_feature 1..714: note = Synthetic Construct
  source 1..714: mol_type = other DNA
caggtgcagctggtgcagtctggggctgaggtgaagaagcctggggcctcagtgaaggtttcctgcaaggcatctggatttaccttcaccgagctgtccatgcactgggtgcgacaggcccctggacaagggcttgagtggatgggaggaatcaaccctaataacggtggcacaagctacaatcagaagttccagggcagagtcaccatgaccagggacacgtccacgagcacagtctacatggagctgagcagcctgagatctgaggacacggccgtgtattactgtgctagagaatcctggggccaaggaaccctggtcaccgtctcctcaggtggcggaggatctggcggaggtggaagcggcggaggcggatctgatattgtgatgacccagtcgccactctctctgcctgtcacccctggagagccggcctccatctcctgcaagtctagtcagagcctcctccactcggatggcaagacctatttgaattggtacctgcagaagccaggccagtctccacagctcctaatctatttagcttcccggcgggcctctggagtgccagataggttcagtggcagcgggtcagggacagatttcacactgaaaatcagccgggtggaggctgaggatgttggggtttattactgctttcaaggtacccactttcctcacactttcggcggagggaccaaggtggagatcaaa

SEQ ID NO: 194 - DNA, 714 bp, synthetic construct
  misc_feature 1..714: note = Synthetic Construct
  source 1..714: mol_type = other DNA
caggtgcagctggtgcagtctggggctgaggtgaagaagcctggggcctcagtgaaggtttcctgcaaggcatctggattcaccttcaccgagtacagtatgcactgggtgcgacaggcccctggacaagggcttgagtggatgggaggcatcaaccctaacaacggtggcacaagctacaaccagaagttccagggcagagtcaccatgaccagggacacgtccacgagcacagtctacatggagctgagcagcctgagatctgaggacacggccgtgtattactgtgctagagagtcctggggccaaggaaccctggtcaccgtctcctcaggtggcggaggatctggcggaggtggaagcggcggaggcggatctgatattgtgatgactcagtctccactctccctgcccgtcacccctggagagccggcctccatctcctgcaaatctagtcagagcctcctgcattcagatgggaagacatatttgaactggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgctttcaaggcacccatttccctcacactttcggcggagggaccaaggtggagatcaaa

SEQ ID NO: 195 - DNA, 714 bp, synthetic construct
  misc_feature 1..714: note = Synthetic Construct
  source 1..714: mol_type = other DNA
caggtgcagctggtgcagtctggggctgaggtgaagaagcctggggcctcagtgaaggtttcctgcaaggcatctggaggcaccttcaccgagtactctatgcactgggtgcgacaggcccctggacaagggcttgagtggatgggaggaatcaaccccaataacggtggcacacgctacaaccagaagttccagggcagagtcaccatgaccagggacacgtccacgagcacagtctacatggagctgagcagcctgagatctgaggacacggccgtgtattactgtgctagagaatcctggggccaaggaaccctggtcaccgtctcctcaggtggcggaggatctggcggaggtggaagcggcggaggcggatctgatattgtgatgacccagtcaccactctctctgccagtcacccctggagagccggcctccatctcctgcaagtctagtcagagcctcctacactccgatggaaagacctatttgaattggtacctgcagaagccaggccagtctccacagctcctaatctatttggtttccaaccgggcctctggagtgccagataggttcagtggcagcgggtcagggacagatttcacactgaaaatcagccgggtggaggctgaggatgttggggtttattactgccaccaaggtactcactttcctcatactttcggcggagggaccaaggtggagatcaaa

SEQ ID NO: 196 - DNA, 714 bp, synthetic construct
  misc_feature 1..714: note = Synthetic Construct
  source 1..714: mol_type = other DNA
caggtgcagctggtgcagtctggggctgaggtgaagaagcctggggcctcagtgaaggtttcctgcaaggcatctggattcaccttcaccgagtactccatgcactgggtgcgacaggcccctggacaagggcttgagtggatgggaggaatcaacccaaacaacggtggcacaagctacaaccagaagttccagggcagagtcaccatgaccagggacacgtccacgagcacagtctacatggagctgagcagcctgagatctgaggacacggccgtgtattactgtgctagagaatcctggggccaaggaaccctggtcaccgtctcctcaggtggcggaggatctggcggaggtggaagcggcggaggcggatctgatattgtgatgacccagagcccactctctctgcccgtcacccctggagaaccggcctccatctcctgcaagtctagtcagagcctcctgcactccgatgggaagacctatttgaattggtacctgcagaagccaggccagtctccacagctcctaatctatctgggctccagccgggcctctggagtgccagataggttcagtggcagcgggtcagggacagatttcacactgaaaatcagccgggtggaggctgaggatgttggggtttattactgcatgcaaggtactcactttcctcatactttcggcggagggaccaaggtggagatcaaa

SEQ ID NO: 197 - DNA, 714 bp, synthetic construct
  misc_feature 1..714: note = Synthetic Construct
  source 1..714: mol_type = other DNA
caggtgcagctggtgcagtctggggctgaggtgaagaagcctggggcctcagtgaaggtttcctgcaaggcatctggattcaccttcaccgaatacagcatgcactgggtgcgacaggcccctggacaagggcttgagtggatgggaggaatcaaccccaataatggtggaacaagctacaatcagaagttccagggcagagtcaccatgaccagggacacgtccacgagcacagtctacatggagctgagcagcctgagatctgaggacacggccgtgtattactgtgctagagaatcctggggccaaggaaccctggtcaccgtctcctcaggtggcggaggatctggcggaggtggaagcggcggaggcggatctgatattgtgatgactcagtctccactctccctgcccgtcacccctggagagccggcctccatctcctgcaagtctagtcagagcctcctgcacaatgatgggaaaacatatttgaattggtacctgcagaagccagggcagtctccacagctcctgatctatttggcttctaatcgggcctccggggtccctgacaggttcagtggcagtggatcaggcacagattttacactgaaaatcagcagagtggaggctgaggatgttggggtttattactgcggccaaggcacgcatttccctcatactttcggcggagggaccaaggtggagatcaaa

SEQ ID NO: 198 - DNA, 714 bp, synthetic construct
  misc_feature 1..714: note = Synthetic Construct
  source 1..714: mol_type = other DNA
caggtgcagctggtgcagtctggggctgaggtgaagaagcctggggcctcagtgaaggtttcctgcaaggcatctggattcaccttcaccgagtactcaatgcactgggtgcgacaggcccctggacaagggcttgagtggatgggagggatcaacccaaacaacggtggaacaagctacaatcagaagttccagggcagagtcaccatgaccagggacacgtccacgagcacagtctacatggagctgagcagcctgagatctgaggacacggccgtgtattactgtgcttccgagtcctggggccaaggaaccctggtcaccgtctcctcaggtggcggaggatctggcggaggtggaagcggcggaggcggatctgatattgtgatgacccagtctccactctctctgccggtcacccctggagaaccggcctccatctcctgcaagtctagtcagagcctcctgcactctgacggaaagacctatttgaactggtacctgcagaagccaggccagtctccacagctcctaatctatctggtttccaagctgaagtctggagtgccagataggttcagtggcagcgggtcagggacagatttcacactgaaaatcagccgggtggaggctgaggatgttggggtttattactgcatgcaaggtacccactttcctcatactttcggcggagggaccaaggtggagatcaaa

SEQ ID NO: 199 - DNA, 714 bp, synthetic construct
  misc_feature 1..714: note = Synthetic Construct
  source 1..714: mol_type = other DNA
caggtgcagctggtgcagtctggggctgaggtgaagaagcctggggcctcagtgaaggtttcctgcaaggcatctggattcaccttcaccgagtactccatgcactgggtgcgacaggcccctggacaagggcttgagtggatgggaggaatcaacccaaacaatggtggaacaaggtacaaccagaagttccagggcagagtcaccatgaccagggacacgtccacgagcacagtctacatggagctgagcagcctgagatctgaggacacggccgtgtattactgtgctagagaatcctggggccaaggaaccctggtcaccgtctcctcaggtggcggaggatctggcggaggtggaagcggcggaggcggatctgatattgtgatgacccagtccccactctctctgcctgtcacccctggagagccggcctccatctcctgcaagtctagtcagagcctccttcacaaggatggtaagacctatttgaactggtacctgcagaagccaggccagtctccacagctcctaatctatctggtttccaaacttccatctggagtgccagataggttcagtggcagcgggtcagggacagatttcacactgaaaatcagccgggtggaggctgaggatgttggggtttattactgcatgcaaggtactcacttccctcatactttcggcggagggaccaaggtggagatcaaa

SEQ ID NO: 200 - DNA, 714 bp, synthetic construct
  misc_feature 1..714: note = Synthetic Construct
  source 1..714: mol_type = other DNA
caggtgcagctggtgcagtctggggctgaggtgaagaagcctggggcctcagtgaaggtttcctgcaaggcatctggatttaccttcaccgagtactcgatgcactgggtgcgacaggcccctggacaagggcttgagtggatgggaggcatcaacccaaacaacggtggcacaatctacaaccagaagttccagggcagagtcaccatgaccagggacacgtccacgagcacagtctacatggagctgagcagcctgagatctgaggacacggccgtgtattactgtgctagagagtcctggggccaaggaaccctggtcaccgtctcctcaggtggcggaggatctggcggaggtggaagcggcggaggcggatctgatattgtgatgactcagtctccactctccctgcccgtcacccctggagagccggcctccatctcctgcaagtctagtcagagcctcttgcactctgatgggaaaacctatttgaattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaggaacccattttcctcacactttcggcggagggaccaaggtggagatcaaa

SEQ ID NO: 201 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggcggcaccttcagcagctacgccatcagctgggtgagacaagcccccggccaaggcctggagtggatgggcgagatcaaccctagcaacggcagaaccgtgtacaatcagaagttccaaggcagagtgaccatgacaagagacacaagcacaagcaccgtgtacatggaactgagcagcctgagaagcgaggacaccgccgtttactactgcgctagatacaaggattattgggggcaaggcaccttggttaccgttagcagcgggggagggggctcgggggggggaggctcgggcggtggcggaagtgatatcgtgatgacacagagccccctgtcgctgcccgtgactcccggcgaacccgcaagcatcagctgcagaagctctcagagcatcgtgaccgccatcggcaacacctacctggagtggtatctgcagaagcccgggcagagccctcagctgctgatctacaaggtgagcaataggttcagcggcgtgcctgatagattcagcggaagcggtagcggcaccgacttcaccctgaagatcagcagagtggaggccgaggacgtgggcgtgtactattgcttccaaggcagccacgtgcccttcaccttcggcggcggcaccaaggtggagatcaag

SEQ ID NO: 202 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggcggcaccttcagcagctacgccatccactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtatattactgcgctagaagaatggactattggggccaaggcaccctggtaacagtatcctccggaggaggaggcagcggcggggggggcagtggcgggggagggtccgatatcgtgatgacacagagccccctgagcctgcccgtgacccccggcgagcccgcaagcatcagctgcagaagcagcaagagcctgctgcacagcaacggcaacacctacctgtactggtatctgcagaagcccgggcagagccctcagctgctgatctacagaatgagcaacagagcaagcggcgtgcccgacagattcagcggcagcggcagcggcaccgacttcaccctgaagatcagcagagtggaggccgaggacgtgggcgtctattactgtatgcaagccctgcagacccccttcaccttcggcggcggcaccaaggtggagatcaag

SEQ ID NO: 203 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtgggtgggcagcatgaaccccaacagcggccacaccggcttcgctcagaagttccaaggcagagtgaccatgacaagagacacaagcacaagcaccgtgtacatggaactgagcagcctgagaagcgaggacaccgccgtatattactgcgctagagctatggactattgggggcaaggcaccctggtgaccgtgtcatccggaggtgggggaagcggcgggggaggctccggcggaggtggcagcgatatcgtgatgacacagagccccctgagcctgcccgtgacccccggcgaacccgcaagcatcagctgcaaaagctctcagagcctgctgcacagcgacggcaagacctacctgtactggtatctgcagaagcccgggcagagccctcagctgctgatctacaaggtgagcaacagattcaagggcgtgcccgacagattcagcggcagcggcagcggcaccgacttcaccctgaagatcagcagagtggaggccgaggacgtgggcgtctattactgtatgcagaagattcagctgcccgtaacatttggcggcggcaccaaggtggagatcaag

SEQ ID NO: 204 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtatattactgctggagatacatggactattggggccaaggcaccctggtaacagtatcctccggaggaggaggcagcggcggggggggcagtggcgggggagggtccgatatcgtgatgacccaaagccccctgagcctacccgtgacccccggcgagcccgcaagcatcagctgcagaagcagcgagagcatcgtgcacagcaacggcaacacctacctggagtggtatctgcagaagcccgggcagagccctcagctgctgatctacaaggtgagcaacaagttcagcggcgtgcccgacagattcagcggcagcggcagcggcaccgacttcaccctgaagatcagcagagtggaggccgaggacgtgggcgtctattactgtatgcagtacacccactggccccccaccttcggcggcggcaccaaggtggagatcaag

SEQ ID NO: 205 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtttactactgctggagatacatggattattgggggcaaggcaccttggttaccgttagcagcgggggagggggctcgggggggggaggctcgggcggtggcggaagtgatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacaaggtggagaataggttcagcggcgtgcctgatagattcagcggaagcggtagcggcaccgacttcaccctgaagatcagcagagtggaggccgaggacgtgggcgtgtactattgtatgcagcacacccactggccccccaccttcggcggcggcaccaaggtggagatcaag

SEQ ID NO: 206 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccccaagaacggcagaaccaactacaatcagaagttccaaggcagagtgaccatgacaagagacacaagcacaagcaccgtgtacatggaactgagcagcctgagaagcgaggacaccgccgtttactactgctggagatacatggattattgggggcaaggcaccttggttaccgttagcagcgggggagggggctcgggggggggaggctcgggcggtggcggaagtgatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacaaggtgagcaataggttcagcggcgtgcctgatagattcagcggaagcggtagcggcaccgacttcaccctgaagatcagcagagtggaggccgaggacgtgggcgtgtactattgcctgcaacacacccactggccccccaccttcggcggcggcaccaaggtggagatcaag

SEQ ID NO: 207 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtttactactgctggagatacatggattattgggggcaaggcaccttggttaccgttagcagcgggggagggggctcgggggggggaggctcgggcggtggcggaagtgatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacgaggtgagcaataggttcagcggcgtgcctgatagattcagcggaagcggtagcggcaccgacttcaccctgaagatcagcagagtggaggccgaggacgtgggcgtgtactattgtatgcagcacacccactggccccccaccttcggcggcggcaccaaggtggagatcaag

SEQ ID NO: 208 - DNA, 720 bp, synthetic construct
  misc_feature 1..720: note = Synthetic Construct
  source 1..720: mol_type = other DNA
caagtgcagctggtccaaagcggtgcagaggtgaagaagcccggcgcaagcgtgaaggtgagctgcaaggcaagcggctacaccttcaccaagtactggatgcactgggtgagacaagcccccggccaaggcctggagtggatgggcgagatcaaccctcagaacggcagaaccaactacaatcagaagttccaaggcagagtgaccatgacaagagacacaagcacaagcaccgtgtacatggaactgagcagcctgagaagcgaggacaccgccgtatattactgctggagatacatggactattgggggcaaggcaccctggtgaccgtgtcaagcggaggtgggggcagcggcggcggaggctccggcggtggtggttccgatatcgtgatgacacagagccccctgagcctgcccgtgacccccggcgagcccgcaagcatcagctgcagaagcagcagaagcctgctgcacagcaacggctacaactacctggactggtatctgcagaagcccgggcagagccctcagctgctgatctacgccgcaagcagcctgcagtcgggggtgcccgacagattcagcggcagcggcagcggcaccgacttcaccctgaagatcagcagagtggaggccgaggacgtgggcgtctattactgcctgcaacacacccactggccccccaccttcggcggcggcaccaaggtggagatcaag

SEQ ID NO: 209 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtttactactgctggagatacatggattattgggggcaaggcaccttggttaccgttagcagcgggggagggggctcgggggggggaggctcgggcggtggcggaagtgatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacaaggtgagcaataggttcagcggcgtgcctgatagattcagcggaagcggtagcggcaccgacttcaccctgaagatcagcagagtggaggccgaggacgtgggcgtgtactattgtatgcagcacacccactggccccccaccttcggcggcggcaccaaggtggagatcaag

SEQ ID NO: 210 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactaccctcagaagttccaaggcagagtgaccatgacaagagacacaagcacaagcaccgtgtacatggaactgagcagcctgagaagcgaggacaccgccgtatattactgctggagatacatggactattggggccaaggcaccctggtaacagtatcctccggaggaggaggcagcggcggggggggcagtggcgggggagggtccgatatcgtgatgacccaaagccccctgagcctacccgtgacccccggcgagcccgcaagcatcagctgcagaagctctcagagcctgctgcacagcaacggctacaactacctggactggtatctgcagaagcccgggcagagccctcagctgctgatctacaaggtgagcaacagattcaccggcgtgcccgacagattcagcggcagcggcagcggcaccgacttcaccctgaagatcagcagagtggaggccgaggacgtgggcgtctattactgtatgcagcacacccactggccccccgccttcggcggcggcaccaaggtggagatcaag

SEQ ID NO: 211 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtttactactgctggagatacatggattattgggggcaaggcaccttggttaccgttagcagcgggggagggggctcgggggggggaggctcgggcggtggcggaagtgatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacaagagcagcaataggttcagcggcgtgcctgatagattcagcggaagcggtagcggcaccgacttcaccctgaagatcagcagagtggaggccgaggacgtgggcgtgtactattgtatgcagcacacccactggccccccaccttcggcggcggcaccaaggtggagatcaag

SEQ ID NO: 212 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtttactactgctggagatacatggattattgggggcaaggcaccttggttaccgttagcagcgggggagggggctcgggggggggaggctcgggcggtggcggaagtgatatcgtgatgacacagagccccctgtcgctgcccgtgactcccggcgaacccgcaagcatcagctgcagaagctctcagagcctgctgaacagcaacggcgtgaccttcgtggagtggtatctgcagaagcccgggcagagccctcagctgctgatctacgaggtgagcaataggttcagcggcgtgcctgatagattcagcggaagcggtagcggcaccgacttcaccctgaagatcagcagagtggaggccgaggacgtgggcgtgtactattgcctgcaacacacccactggccccccaccttcggcggcggcaccaaggtggagatcaag

SEQ ID NO: 213 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcgagatcaaccctagcaacggcagaaccaactacaatcagaagttccaaggcagagtgaccatgacaagagacacaagcacaagcaccgtgtacatggaactgagcagcctgagaagcgaggacaccgccgtatattactgctggagatacatggactattggggccaaggcaccctggtaacagtatcctccggaggaggaggcagcggcggggggggcagtggcgggggagggtccgatatcgtgatgacccaaagccccctgagcctacccgtgacccccggcgagcccgcaagcatcagctgcagaagctctcagagcctgctgcacagcaacggctacaactacctggactggtatctgcagaagcccgggcagagccctcagctgctgatctacgaggtgagcaacagattctggggcgtgcccgacagattcagcggcagcggcagcggcaccgacttcaccctgaagatcagcagagtggaggccgaggacgtgggcgtctattactgcctgcaacacacccactggccccccaccttcggcggcggcaccaaggtggagatcaag

SEQ ID NO: 214 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcaccaagtactggatgcactgggtgagacaagcccccggccaaggcctggagtggatgggcggcatcaaccctagcaacggcagaaccaactacaatcagaagttccaaggcagagtgaccatgacaagagacacaagcacaagcaccgtgtacatggaactgagcagcctgagaagcgaggacaccgccgtatattactgcgctagatacatgcagtattggggccaaggcaccctggtaacagtatcctccggaggaggaggcagcggcggggggggcagtggcgggggagggtccgatatcgtgatgacacagagccccctgagcctgcccgtgacccccggcgagcccgcaagcatcagctgcagaagcagcaagagcctgctgcacagcaacggcaacacctacctgtactggtatctgcagaagcccgggcagagccctcagctgctgatctacaaggtgagcaatcagagcagcggcgtgcccgacagattcagcggcagcggcagcggcaccgacttcaccctgaagatcagcagagtggaggccgaggacgtgggcgtctattactgtatgcaagccctgcagacccccctgaccttcggcggcggcaccaaggtggagatcaag

SEQ ID NO: 215 - DNA, 720 bp, synthetic construct
  misc_feature 1..720: note = Synthetic Construct
  source 1..720: mol_type = other DNA
caagtgcagctggtgcagagcggcgccgaggtgaagaagcccggcgcaagcgtgaaggtgagctgcaaggcaagcggctacaccttcacaagccactacctgcactgggtgagacaagcccccggccaaggcctggagtggatgggcatcatcaaccctagcaacggcagaaccaactacaatcagaagttccaaggcagagtgaccatgacaagagacacaagcacaagcaccgtgtacatggaactgagcagcctgagaagcgaggacaccgccgtttactactgctggagatacatggattattgggggcaaggcaccttggttaccgttagcagcgggggagggggctcgggggggggaggctcgggcggtggcggaagtgatatcgtgatgacacagagccccctgtcgctgcccgtgactcccggcgaacccgcaagcatcagctgcagaagctctcagagcctgctgcacagcaacggctacaactacctggactggtatctgcagaagcccgggcagagccctcagctgctgatctacgaggtgagcaataggttcagcggcgtgcctgatagattcagcggaagcggtagcggcaccgacttcaccctgaagatcagcagagtggaggccgaggacgtgggcgtgtactattgcctgcaacacacccactggccccccaccttcggcggcggcaccaaggtggagatcaag

SEQ ID NO: 216 - protein, 724 aa, synthetic construct
  REGION 1..724: note = Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 217 - protein, 725 aa, synthetic construct
  REGION 1..725: note = Synthetic Construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 218 - protein, 25 aa, synthetic construct
  REGION 1..25: note = Synthetic Construct
NAGSGSGFNGGFGSSMDSKSSGWGM

SEQ ID NO: 219 - protein, 28 aa, synthetic construct
  REGION 1..28: note = Synthetic Construct
NAGSGSGFNGGFGSSMDSKPSERSGWGM

SEQ ID NO: 220 - protein, 28 aa, synthetic construct
  REGION 1..28: note = Synthetic Construct
NAGSGSGFNGGFGSSMDSKSPSERGWGM

SEQ ID NO: 221 - protein, 31 aa, synthetic construct
  REGION 1..31: note = Synthetic Construct
NAGSGSGFNGGFGSSMDSKPSERPSERGWGM